(19) United States
(12) Patent Application Publication
(10) Pub. No.: US 2024/0390238 A1
(43) Pub. Date: Nov. 28, 2024

(54) MINIMALIST EMULSION COMPOSITIONS

(71) Applicant: Baek Clinical Inc., Los Angeles, CA (US)

(72) Inventor: Jihoon P. BAEK, Los Angeles, CA (US)

(21) Appl. No.: 18/696,088

(22) PCT Filed: Sep. 27, 2022
(86) PCT No.: PCT/US22/77119

§ 371 Date: Mar. 27, 2024

Related U.S. Application Data
(60) Provisional application No. 63/248,892, filed on Sep. 27, 2021.
(60) Provisional application No. 63/248,993, filed on Sep. 27, 2021.

(51) Int. Cl. A61K 8/06; A61K 8/34; A61K 8/37; A61K 8/67; A61Q 19/00; A61Q 19/08

(52) U.S. Cl. CPC A61K 8/062; A61K 8/345; A61K 8/375; A61K 8/673; A61K 8/676; A61Q 19/007; A61Q 19/08

(57) ABSTRACT

Topical formulations of oil-in-water emulsions, composed of water, panthenol, alkanediol(s), chelating agent(s), barrier-repairing agent(s), emollient ester(s), and soothing agent(s) are provided. The formulations are storage stable for an extended period of time without significant separation of the emulsion or changes in texture, appearance, odor or pH, and have desirable physical properties. Topical compositions of this disclosure find use in treating or preventing a variety of cosmetic and/or dermatological conditions. Aspects of the present disclosure include compositions and kits comprising a minimalist hydrating formulation. Aspects of the present disclosure include compositions and kits comprising a vitamin C formulation. Aspects of the present disclosure include a kit comprising a minimalist hydrating formulation and a vitamin C formulation.

### CROSS-REFERENCE TO RELATED APPLICATIONS

This application claims the benefit of U.S. Provisional Patent Application Nos. 63/248,892, filed Sep. 27, 2021, and 63/248,993, filed Sep. 27, 2021, each of which applications is incorporated herein by reference in its entirety.

### FIELD OF INVENTION

Compositions, kits, and methods for treating, preventing, or improving dermatocosmetic conditions.

### INTRODUCTION

Emulsions, composed of a water and oil/silicone phase, are a useful and convenient vehicle in which to delivery water- and oil-soluble ingredients that prevent moisture loss from skin, or trans epidermal water loss (TEWL), which is often associated with a range of cosmetic and/or dermatological conditions associated with dry and scaly skin, such as dermatitis, psoriasis, xerosis, ichthyosis, and eczema.

In conditions in which skin barrier function has been severely compromised, skin often displays increased sensitivity, with reactions of skin redness and stinging in response to topical emulsions that do not induce such reactions in non-compromised skin. It has been suggested that several components typical of emulsions currently available may contribute to this, including certain emulsifiers, stabilizers and emollients.

Many of the aforementioned ingredients are included to aid in the sensorial properties and shelf stability of the end product, such as creating a smooth, viscous, uniform texture that is pleasant to use on the skin. These properties are integral to user compliance and satisfaction, separate from the longer-term moisturizing and skin-conditioning properties of the product.

There has been and remains a need for emulsions that are free of ingredients that are irritating to sensitive skin yet retain pleasant sensorial properties and provide a shelf and storage-stable product.

### SUMMARY

Topical formulations of an oil-in-water emulsion, composed of water, panthenol, alkanediol(s), chelating agent(s), barrier-repairing agent(s), emollient ester(s), and soothing agent(s) are provided. The topical formulations have desirable physical properties. The topical formulations can include concentrations of water of 55% to 80% by weight, panthenol of 3% to 10% by weight, alkanediol(s) of 4% to 8% by weight, chelating agent(s) of 0.05% to 0.2% by weight, barrier-repairing agents of 2% to 10% by weight, emollients of 2% to 18% by weight, emulsifier of 0.5% to 3% by weight, soothing agent of 0.1% to 1.5% by weight, and less than 0.5% by weight each of the following: fatty alcohol stabilizer(s), triglyceride(s), and silicone oil(s). Topical compositions of this disclosure find use in treating or preventing a variety of cosmetic and/or dermatological conditions.

Topical formulations of vitamin C (L-ascorbic acid) dissolved in a combination of a urea agent and a non-aqueous skin-compatible solvent are storage stable for an extended period of time without significant degradation of the L-ascorbic acid in the composition, are have been found to have desirable physical properties. Such topical compositions of vitamin C have found use in treating or preventing a variety of cosmetic and/or dermatological conditions as well as to reduce the appearance of chronological and/or environmentally-caused skin aging.

There has been and remains a need for formulation combinations and daily regimens that are effective in reducing the effects of skin aging, yet free of ingredients that are irritating to sensitive skin yet retain pleasant sensorial properties.

In one embodiment, disclosed herein there is a storage stable topical emulsion composition, comprising:
a continuous phase that is at least 75% by weight in total of the composition, comprising:
a. a solvent comprising a mixture of water and an alkanediol; and
b. panthenol;
a dispersed phase that is at least 5% by weight in total, comprising:
a. a non-ethoxylated emulsifier at 0.5% to 3% by weight;
b. an emollient;
c. a barrier repairing agent; and
d. a soothing agent;
with the proviso that the composition comprises:
e. less than 0.5% by weight of oil-based stabilizers (e.g., fatty alcohol stabilizer, e.g., cetyl alcohol) and
f. less than 0.5% by weight of triglycerides; and
g. less than 0.5% by weight of ethoxylated emulsifiers,

In some embodiments, the composition is storage stable and exhibits a viscosity ranging from 10,000 to 75,000 cps (e.g., 10,000+/−1,000 cps) and pH between 3.5 and 6.5 (e.g., 5.0 to 6.0) when stored at 25° C.

In some embodiments, the composition is storage stable and exhibits a viscosity ranging from 15,000 to 75,000 cps (e.g., 15,000+/−1,000 cps) and pH between 3.5 and 6.5 (e.g., 5.0 to 6.0) when stored at 25° C.

In some embodiments, the composition is storage stable and exhibits a viscosity ranging from 40,000 to 75,000 cps (e.g., 45,000+/−1,000 cps) and pH between 4.0 and 6.0 (e.g., 5.0 to 6.0) when stored at 25° C.

In certain embodiments, the composition comprises:
a. 55%-80% by weight of water;
b. 4% to 8% by weight of the alkanediol;
c. 3% to 10% by weight of panthenol;
d. 0.5% to 3% by weight of the non-ethoxylated emulsifier (e.g., lecithin, sodium acrylate copolymer, etc.);
e. 2%-18% by weight of the emollient;
f. 2%-10% by weight of the barrier repairing agent;
g. 0.1% to 1.5% by weight of the soothing agent;
with the proviso that the composition does not comprise an oil-based stabilizer or a triglyceride.

In certain embodiments, the composition:
is storage stable against separation of continuous and dispersed phases;
is capable of application to the skin of a subject without pilling;
is capable of providing a cooling sensation on the skin of a subject;
exhibits a viscosity of 45,000-75,000 cps and pH between 5.0 and 6.0 when stored at 25° C. for 3 months.

In certain embodiments, the alkanediol is selected from propanediol, propylene glycol, butylene glycol, pentylene glycol, hexanediol, caprylyl glycol, and a combination thereof. In certain cases, the continuous phase comprises 1,2-hexanediol, and propanediol. In certain cases, the continuous phase comprises propanediol and caprylyl glycol. In certain cases, the alkanediol is 1,3 propanediol. In some embodiments, the composition comprises about 6%-7% by weight of 1,3 propanediol.

In some embodiments, the composition comprises 65% to 71% by weight of water.

In some embodiments, the composition comprises about 5% by weight of panthenol. In certain cases, the composition comprises from 3.5% to 5.5% by weight of panthenol.

In some embodiments, the composition (e.g., continuous phase of the composition) further comprises a chelating agent. In certain cases, the composition comprises 0.05% to 0.2% by weight of the chelating agent. In certain cases, the chelating agent is phytic acid or EDTA, or a salt thereof (e.g., disodium EDTA, or sodium phytate). In certain embodiments, the continuous phase comprises about 0.1% by weight of phytic acid solution (50% (w/w) in water).

In some embodiments, the barrier repairing agent is selected from c10-30 cholesterol/lanosterol esters, cholesteryl isostearate, cholesteryl chloride, cholesteryl nonanoate, lanolin, lanolin wax, lanolin oil, lanolin acid, lanolin alcohol, and a combination thereof. In certain cases, the composition comprises about 4% by weight of C10-30 cholesterol/lanosterol esters.

In some embodiments, the emollient is selected from pentaerythritol tetraethylhexanoate, c12-15 alkyl benzoate, dipantaerythrityl pentaisononanoate, pentaerythrityl tetraisostearate, pentaerythrityl tetrabehenate, dipentaerythrityl pentaisononanoate, diisopropyl sebacate, isononyl isononanoate, squalane, and a combination thereof. In certain cases, the composition comprises about 12% by weight of pentaerythrityl tetraethylhexanoate.

In some embodiments, the emulsifier is selected from lecithin, sodium acrylate copolymer, and a combination thereof. In certain cases, the composition comprises a blend of lecithin and sodium acrylate copolymer. In certain cases, the composition comprises about 2% by weight of the blend of lecithin and sodium acrylate copolymer.

In some embodiments, the soothing agent is selected from: centella asiatica extract, madecassoside, asiaticoside, glycyrrhetinic acid, and a combination thereof. In some cases, the composition comprises a blend of madecassoside, asiaticoside; and glycyrrhetinic acid. In certain cases, the composition comprises about 0.14% by weight madecassoside and about 0.06% by weight asiaticoside. In certain cases, the composition comprises about 0.2% by weight blend of madecassoside and asiaticoside, and about 0.5% by weight glycyrrhetinic Acid.

In some embodiments, the composition comprises a preservative, wherein the preservative is an antimicrobial agent. In certain cases, the antimicrobial agent is caprylhydroxamic acid. In some cases, the continuous phase further comprises caprylhydroxamic acid (e.g., about 0.05-0.15% by weight). In some embodiments, the continuous phase comprises caprylhydroxamic acid, 1,2-hexanediol, and propanediol. In certain cases, the continuous phase comprises about 0.05% by weight of caprylhydroxamic acid, about 0.30% by weight of 1,2-hexanediol, and about 6.65% by weight propanediol.

In some embodiments, the fatty alcohol stabilizer is selected from cetyl alcohol, cetearyl alcohol, behenyl alcohol, stearyl alcohol, and a combination thereof.

In some embodiments, the triglyceride is selected from: caprylic/capric triglyceride, argan oil, sunflower seed oil, rapeseed oil, olive oil, coconut oil, palm kernel oil, babassu oil, palm oil, apricot kernel oil, tallow, baobab oil, cocoa butter, andiroba seed oil, mango butter, avocado oil, cottonseed oil, marula oil, shea butter, grape seed oil, almond oil, safflower oil, moringa oil, and a combination thereof.

In some embodiments, the composition exhibits a pH ranging from 3.5 to 6.0 after storage for at least 3 months at 5° C.±2° C. in a multi-use container. In some cases, the composition exhibits a pH ranging from 3.5 to 6.0 after storage for at least 3 months at 25° C.±2° C. in a multi-use container. In some cases, the composition exhibits a pH ranging from 3.5 to 6.0 after storage for at least 3 months at 40° C.±2° C. in a multi-use container. In some cases, the composition exhibits a pH ranging from 3.5 to 6.0 after storage for at least 3 months at 45° C.±2° C. in a multi-use container.

In some embodiments, the composition exhibits a viscosity ranging from 15,000 to 75,000 cps after storage for at least 3 months at 5° C.±2° C. in a multi-use container. In some cases, the composition exhibits a viscosity ranging from 15,000 to 75,000 cps after storage for at least 3 months at 25° C.±2° C. in the multi-use container. In some cases, the composition exhibits a viscosity ranging from 15,000 to 75,000 cps after storage for at least 3 months at 40° C.±2° C. in the multi-use container. In some cases, the composition exhibits a viscosity ranging from 15,000 to 75,000 cps after storage for at least 3 months at 45° C.±2° C. in the multi-use container.

In some embodiments, the composition exhibits a viscosity ranging from 20,000 to 75,000 cps after storage for at least 3 months at 5° C.±2° C. in a multi-use container. In some cases, the composition exhibits a viscosity ranging from 20,000 to 75,000 cps after storage for at least 3 months at 25° C.±2° C. in the multi-use container. In some cases, the composition exhibits a viscosity ranging from 20,000 to 75,000 cps after storage for at least 3 months at 40° C.±2° C. in the multi-use container. In some cases, the composition exhibits a viscosity ranging from 20,000 to 75,000 cps after storage for at least 3 months at 45° C.±2° C. in the multi-use container.

In some embodiments, the composition exhibits a viscosity ranging from 40,000 to 75,000 cps after storage for at least 3 months at 5° C.±2° C. in a multi-use container. In some cases, the composition exhibits a viscosity ranging from 40,000 to 75,000 cps after storage for at least 3 months at 25° C.±2° C. in the multi-use container. In some cases, the composition exhibits a viscosity ranging from 40,000 to 75,000 cps after storage for at least 3 months at 40° C.±2° C. in the multi-use container. In some cases, the composition exhibits a viscosity ranging from 40,000 to 75,000 cps after storage for at least 3 months at 45° C.±2° C. in the multi-use container.

In some embodiments, the composition exhibits a viscosity ranging from 49,000 to 53,000 for at least 3 months at 5° C.±2° C. in the multi-use container. In some embodiments, the composition exhibits a viscosity ranging from 49,000 to 55,000 for at least 3 months at 25° C.±2° C. in the multi-use container. In some embodiments, the composition exhibits a viscosity ranging from 40,000 to 52,500 for at least 3 months at 40° C.±2° C. in the multi-use container. In some embodiments, the composition exhibits a viscosity ranging from 40,000 to 51,500 for at least 3 months at 45° C.±2° C. in the multi-use container.

In some embodiments the composition is stored in a multi-use container, and the multi-use container is a glass bottle.

In some embodiments, the composition is stored in a multi-use container, and the multi-use container comprises a plastic bottle with an airless pump.

Aspects of the present disclosure include A storage stable topical emulsion composition, comprising: a continuous phase that is at least 75% by weight in total of the composition, comprising: a solvent comprising a mixture of water and an alkanediol; and panthenol; a dispersed phase that is at least 5% by weight in total, comprising: a non-ethoxylated emulsifier at 0.5% to 3% by weight; an emollient; a barrier repairing agent; and a soothing agent;

with the proviso that the composition comprises: less than 0.5% by weight of oil-based stabilizers (e.g., fatty alcohol stabilizer, e.g., cetyl alcohol) and less than 0.5% by weight of triglycerides; and less than 0.5% by weight of ethoxylated emulsifiers, the dispersed phase is at least 5% by weight in total. the dispersed phase is at least 10% by weight in total. 2% by weight of the non-ethoxylated emulsifier. comprising 3% by weight of the non-ethoxylated emulsifier.

In some embodiments, the composition is storage stable and exhibits a viscosity ranging from 10,000 to 75,000 cps. In some embodiments, the composition exhibits a pH between 3.5 and 6.5 (e.g., 5.0 to 6.0) when stored at 25° C. In some embodiments, the composition exhibits a pH between 3.5 and 6.5 (e.g., 5.0 to 6.0) when stored at 30° C. In some embodiments, the composition exhibits a pH between 3.5 and 6.5 (e.g., 5.0 to 6.0) when stored at 35° C.

In some embodiments, the composition comprises: 55%-80% by weight of water; 4% to 8% by weight of the alkanediol; 3% to 10% by weight of panthenol; 0.5% to 3% by weight of the non-ethoxylated emulsifier (e.g., lecithin, sodium acrylate copolymer, etc.); 2%-18% by weight of the emollient; 2%-10% by weight of the barrier repairing agent; 0.1% to 1.5% by weight of the soothing agent; with the proviso that the composition does not comprise an oil-based stabilizer or a triglyceride. In some embodiments, the composition: is storage stable against separation of continuous and dispersed phases; is capable of application to the skin of a subject without pilling; is capable of providing a cooling sensation on the skin of a subject; exhibits a viscosity of 20,000-75,000 cps and pH between 5.0 and 6.5 when stored at 25° C. for 3 months.

In some embodiments, the composition exhibits a viscosity of 15,000-75,000 cps. the composition: is storage stable against separation of continuous and dispersed phases; is capable of application to the skin of a subject without pilling; is capable of providing a cooling sensation on the skin of a subject.

In some embodiments, the composition exhibits a viscosity of 45,000-75,000 cps and pH between 5.0 and 6.0 when stored at 25° C. for 3 months.

In some embodiments, the composition the alkanediol is selected from propanediol, propylene glycol, butylene glycol, pentylene glycol, hexanediol, caprylyl glycol, and a combination thereof.

In some embodiments, the composition the continuous phase comprises 1,2-hexanediol, and propanediol. In some embodiments, the composition the continuous phase comprises propanediol and caprylyl glycol. In some embodiments, the composition the alkanediol is 1,3 propanediol. In some embodiments, the composition the composition comprises about 6%-7% by weight of 1,3 propanediol. In some embodiments, the composition the composition comprises 65% to 71% by weight of water. In some embodiments, the composition, the composition comprises about 5% by weight of panthenol. In some embodiments, the composition the composition (e.g., continuous phase of the composition) further comprises a chelating agent. In some embodiments, the composition the composition comprises 0.05% to 0.2% by weight of the chelating agent. In some embodiments, the composition the chelating agent is phytic acid or EDTA, or a salt thereof (e.g., disodium EDTA, or sodium phytate).

In some embodiments, the composition the continuous phase comprises about 0.1% by weight of phytic acid solution (50% (w/w) in water). In some embodiments, the composition the barrier repairing agent is selected from c10-30 cholesterol/lanosterol esters, cholesteryl isostearate, cholesteryl chloride, cholesteryl nonanoate, lanolin, lanolin wax, lanolin oil, lanolin acid, lanolin alcohol, and a combination thereof. In some embodiments, the composition the composition comprises about 4% by weight of C10-30 cholesterol/lanosterol esters. the emollient is selected from pentaerythritol tetraethylhexanoate, c12-15 alkyl benzoate, dipantaerythrityl pentaisononanoate, pentaerythrityl tetraisostearate, pentaerythrityl tetrabehenate, dipentaerythrityl pentaisononanoate, diisopropyl sebacate, isononyl isononanoate, squalane, and a combination thereof.

In some embodiments, the composition comprises about 12% by weight of pentaerythrityl tetraethylhexanoate. In some embodiments, the composition the emulsifier is selected from lecithin, sodium acrylate copolymer, and a combination thereof.

In some embodiments, the composition comprises a blend of lecithin and sodium acrylate copolymer. In some embodiments, the composition comprises about 2% by weight of the blend of lecithin and sodium acrylate copolymer.

In some embodiments, the soothing agent is selected from: centella asiatica extract, madecassoside, asiaticoside, glycyrrhetinic acid, and a combination thereof. In some embodiments, the composition comprises a blend of madecassoside, asiaticoside; and glycyrrhetinic acid.

In some embodiments, the composition comprises about 0.14% by weight madecassoside and about 0.06% by weight asiaticoside. In some embodiments, the composition comprises about 0.2% by weight blend of madecassoside and asiaticoside, and about 0.5% by weight glycyrrhetinic Acid.

In some embodiments, the composition comprises a preservative, wherein the preservative is an antimicrobial agent. In some embodiments, the antimicrobial agent is caprylhydroxamic acid.

In some embodiments, the continuous phase further comprises caprylhydroxamic acid (e.g., about 0.05-0.15% by weight). In some embodiments, the continuous phase comprises caprylhydroxamic acid, 1,2-hexanediol, and propanediol.

In some embodiments, the continuous phase comprises about 0.05% by weight of caprylhydroxamic acid, about 0.30% by weight of 1,2-hexanediol, and about 6.65% by weight propanediol. In some embodiments, the fatty alcohol stabilizer is selected from cetyl alcohol, cetearyl alcohol, behenyl alcohol, stearyl alcohol, and a combination thereof.

In some embodiments, the triglyceride is selected from: caprylic/capric triglyceride, argan oil, sunflower seed oil, rapeseed oil, olive oil, coconut oil, palm kernel oil, babassu oil, palm oil, apricot kernel oil, tallow, baobab oil, cocoa butter, andiroba seed oil, mango butter, avocado oil, cottonseed oil, marula oil, shea butter, grape seed oil, almond oil, safflower oil, moringa oil, and a combination thereof.

In some embodiments, the composition exhibits a pH ranging from 3.5 to 6.5 after storage for at least 3 months at 5° C.±2° C. in a multi-use container. In some embodiments, the composition exhibits a pH ranging from 3.5 to 6.5 after storage for at least 3 months at 25° C.±2° C. in a multi-use container. In some embodiments, the composition exhibits a pH ranging from 3.5 to 6.5 after storage for at least 3 months at 40° C.±2° C. in a multi-use container. In some embodiments, the composition exhibits a pH ranging from 3.5 to 6.5 after storage for at least 3 months at 45° C.±2° C. in a multi-use container. In some embodiments, the composition exhibits a viscosity ranging from 10,000 to 75,000 cps after storage for at least 3 months at 5° C.±2° C. in a multi-use container. In some embodiments, the composition exhibits a viscosity ranging from 10,000 to 75,000 cps after storage for at least 3 months at 25° C.±2° C. in the multi-use container. In some embodiments, the composition exhibits a viscosity ranging from 10,000 to 75,000 cps after storage for at least 3 months at 40° C.±2° C. in the multi-use container. In some embodiments, the composition exhibits a viscosity ranging from 10,000 to 75,000 cps after storage for at least 3 months at 45° C.±2° C. in the multi-use container. In some embodiments, the composition exhibits a viscosity ranging from 49,000 to 53,000 for at least 3 months at 5° C.±2° C. in the multi-use container. In some embodiments, the composition exhibits a viscosity ranging from 49,000 to 55,000 for at least 3 months at 25° C.±2° C. in the multi-use container. In some embodiments, the composition exhibits a viscosity ranging from 40,000 to 52,500 for at least 3 months at 400° C.±2° C. in the multi-use container. In some embodiments, the composition exhibits a viscosity ranging from 40,000 to 51,500 for at least 3 months at 45° C.±2° C. in the multi-use container.

In some embodiments, the multi-use container is a glass bottle. In some embodiments, the multi-use container comprises a plastic bottle with an airless pump.

Another aspect of the present disclosure provides a kit comprising a topical vitamin C formulation of the present disclosure. In some embodiments, the kit comprises instructions for using the vitamin C formulation. In some embodiments, the vitamin C formulation is a formulation described in one or both of International Application publication Number WO2020081868; and International Application Number WO2021212075, which applications are incorporated herein by reference in their entireties.

Another aspect of the present disclosure provides a kit comprising a minimalist hydrating formulation of the present disclosure. In some embodiments, the kit comprises instructions for using the minimalist hydrating formulation

Another aspect of the present disclosure provides a kit comprising a topical vitamin C formulation and a topical minimalist hydrating formulation. In some embodiments, the vitamin C formulation is a formulation described in one or both of International Application publication Number WO2020081868; and International Application Number WO2021212075, which applications are incorporated herein by reference in their entireties. Also provided herein are methods for using the kit as part of a daily regimen. In certain embodiments, the topical vitamin C formulation and the topical minimalist hydrating formulation work together synergistically to provide significant improvements in skin aging such as wrinkling, dark spots, firmness and elasticity. In some embodiments, the kit comprises packaging for the vitamin C formulation and minimalist hydration formulation. The present disclosure provides a kit comprising a package containing i) a topical vitamin C formulation (e.g., as described herein); ii) a topical minimalist hydrating formulation (e.g., as described herein; and iii) instructions for use.

In accordance with embodiments of the invention, the kit comprises at least two containers (e.g., as described herein).

In accordance with the invention, the kit comprises instructions (e.g., a leaflet) inserted into the container or box, typically a user information leaflet containing printed information, which information may include a description of the form and composition of the formulations contained in the kit, an indication of the use for which the product is intended, instructions as to how the product is to be used (e.g., with respect to each of the two formulations) and information and warnings concerning adverse effects and contraindications associated with the use. In accordance with the present disclosure, the leaflet will usually contain the information concerning the uses, treatment regimens, etc. as described herein in relation to the methods of use of the present disclosure. In certain cases, the leaflet contains printed instructions to repeatedly (self-)apply the topical formulations in order to reduce skin aging effects such as wrinkling, dark spots, firmness and elasticity.

In some embodiments, the vitamin C formulation comprises:
a. 5% to 28% by weight ascorbic acid;
b. 5% to 20% by weight of a urea agent;
c. 0.1% to 5% by weight of a cinnamic acid or derivative thereof; and
d. less than 10% by weight in total of one or more optional additional components;
e. dissolved in a non-aqueous skin-compatible solvent comprising polyol, C₍₂₋₆₎ alkanediol, glycol ether, dimethyl ether, or a combination thereof; wherein the ascorbic acid is dissolved at a concentration (AA) that is above its maximum concentration in the solvent alone (X), and the urea is dissolved at a concentration that is at least (AA-X)*1.25.

In some embodiments, the minimalist hydrating formulation comprises:
a continuous phase that is at least 75% by weight in total of the composition, comprising:
f. a solvent comprising a mixture of water and an alkanediol; and
g. panthenol;
a dispersed phase that is at least 5% by weight in total, comprising:
h. a non-ethoxylated emulsifier at 0.5% to 3% by weight;
i. an emollient;
j. a barrier repairing agent; and
k. a soothing agent;
with the proviso that the composition comprises:
l. less than 0.5% by weight of oil-based stabilizers (e.g., fatty alcohol stabilizer, e.g., cetyl alcohol) and
m. less than 0.5% by weight of triglycerides; and
n. less than 0.5% by weight of ethoxylated emulsifiers.

In some embodiments, the composition of the minimalist hydrating formulation is storage stable and exhibits a viscosity ranging from 10,000 to 75,000 cps (e.g., 45,000+/−1,000 cps) and pH between 3.5 and 6.5 (e.g., 5.0 to 6.0) when stored at 25° C.

In some embodiments, the minimalist emulsion formulation comprises:
h. 55%-80% by weight of water;
i. 4% to 8% by weight of the alkanediol;
j. 3% to 10% by weight of panthenol;
k. 0.5% to 3% by weight of the non-ethoxylated emulsifier (e.g., lecithin, sodium acrylate copolymer, etc);
l. 2%-18% by weight of the emollient;
m. 2%-10% by weight of the barrier repairing agent;
n. 0.1% to 1.5% by weight of the soothing agent;
with the proviso that the composition does not comprise an oil-based stabilizer or a triglyceride.

In some embodiments, the kit further comprises instructions for using the minimalist hydrating formulation and the vitamin C formulation. In certain embodiments, the instructions for using the minimalist hydrating formulation comprise: applying an effective amount of a topical minimalist hydrating formulation to an area of the skin.

In certain embodiments, the instructions for using the vitamin C formulation comprise applying an effective amount of a vitamin C formulation to the same area of the skin for which the minimalist hydrating formulation was applied.

In certain embodiments, the area of skin is selected from: face, arms, and legs. In certain embodiments, the instructions comprise:
(a) gently applying ½ to 1 pump of the vitamin C formulation to clean skin;
(b) applying the vitamin C formulation in the morning or evening, before or after the use of the minimalist hydrating formulation

In some embodiments, the instructions further comprise using the vitamin C formulation 2-3 times per week, once daily, or initially 2-3 times per week until the skin is acclimated to the vitamin C formulation, followed by once daily.

In some embodiments, the instructions further comprise applying the minimalist hydrating formulation in the morning or evening, once daily or as needed.

In some embodiments, vitamin C formulation is used before applying the minimalist hydrating formulation.

In some embodiments, vitamin C formulation is used after applying the minimalist hydrating formulation. In some embodiments, the vitamin C formulation comprises: Propanediol, Ascorbic Acid, Urea, Ferulic Acid, Diglycerin, and Pinus Pinaster Bark Extract.

In some embodiments, the minimalist hydrating formulation comprises:
Water, Pentaerythrityl Tetraethylhexanoate, Propanediol, Panthenol, C10-30 Cholesterol/Lanosterol Esters, Sodium Acrylates Copolymer, Lecithin, Glycyrrhetinic Acid, Madecassoside, Asiaticoside, Phytic acid, Caprylhydroxamic acid, and 1,2-Hexanediol.

In some embodiments, the kit comprises two containers, wherein one container comprises the vitamin C formulation, and a second container comprises the minimalist hydrating formulation.

Aspects of the present disclosure include a method of reducing the appearance of skin aging, the method comprising:
i) applying an effective amount of a topical minimalist hydrating formulation of any one of claims 1-59 to an area of skin; and
ii) applying an effective amount of a vitamin C formulation to the same area of skin as the minimalist hydrating formulation;
wherein steps i) and ii) are carried out at least once daily to reduce the appearance of skin aging.

In some embodiments, the vitamin C formulation is applied immediately after or immediately before applying the minimalist hydrating formulation. The method of claim 78,
wherein the vitamin C formulation is applied 1 minute after applying the minimalist hydrating formulation, 2 minutes after applying the minimalist hydrating formulation, 3 minutes after applying the minimalist hydrating formulation, 4 minutes after applying the minimalist hydrating formulation, 5 minutes after applying the minimalist hydrating formulation, 6 minutes after applying the minimalist hydrating formulation, 7 minutes after applying the minimalist hydrating formulation, 8 minutes after applying the minimalist hydrating formulation, 9 minutes after applying the minimalist hydrating formulation, or 10 minutes after applying the minimalist hydrating formulation.

In some embodiments, each of the minimalist hydrating formulation and the vitamin C formulation are applied at once daily, at least twice daily, or 2-3 times per week. Aspects of the present disclosure include methods for using the minimalist hydrating formulation of the present disclosure. Aspects of the present disclosure include methods for using the vitamin C formulation of the present disclosure.

### BRIEF DESCRIPTION OF THE DRAWINGS

FIG. 1 provides a microscopy image of a subject before treatment with formulation 1 of the minimalist hydration formulation of the present application (panel A), and after treatment with formulation 1 of the minimalist hydration formulation of the present application (panel B). After treatment with formulation 1, a marked improvement in skin texture and dryness was observed after 14 days of treatment. Images were taken with LED UV-A light video camera with high resolution to study the skin surface directly.

FIG. 2 provides a histogram illustrating expert grading assessments of skin improvements of subjects after treatment with formulation 1 of the minimalist hydration formulation of the present application. Subjects were observed for visual (hydration, radiance and texture) improvements on the face skin and lower leg skin, and for tactile (softness) improvements on the face skin before (TO) and after 14 (T14) days of treatment. Shown are mean and standard error per time-point.

FIG. 3 provides a histogram illustrating Corneometer CM 825 measurements on skin improvements before (TO) and after 14 (T14) days of treatment of formulation 1 of the minimalist hydration formulation. Treatment with formulation 1 significantly increases hydration on the skin. Shown are mean and standard error per time-point.

FIG. 4 provides a histogram illustrating Visioscan VC 20plus measurements on skin improvements before (TO) and after 14 (T14) days of treatment of formulation 1 of the minimalist hydration formulation. Treatment with formulation 1 significantly reduces scaliness and dryness on the skin. Shown are mean and standard error per time-point.

FIGS. 5A-5B provide histograms illustrating results of a self-assessment questionnaire about skin improvements on the legs performed by the study subjects in a clinical trial. Treatment with formulation 1 i of the minimalist hydration formulation improves skin hydration, softness, smoothness, radiance, and texture. The assessment attributes and scale are summarized in Table 19, and the results are summarized in Tables 22.

FIGS. 6A-6B provide histograms illustrating results of a self-assessment questionnaire about skin improvements on the face performed by the study subjects in a clinical trial. Treatment with formulation 1 of the minimalist hydration formulation improves skin hydration, softness, smoothness, radiance, and texture. The assessment attributes and scale are summarized in Table 19, and the results are summarized in Tables 23.

FIG. 7 depicts a microscopy image of a representative subject before treatment with formulation 1 (minimalist hydration formulation 1) of the present application (TO), and after treatment with minimalist hydration formulation 1 of the present application (T14). After treatment with minimalist hydration formulation 1, a marked improvement in skin hydration, texture and dryness was observed after 14 days of treatment. Images were taken with LED UV-A light video camera with high resolution to study the skin surface directly.

FIG. 8 illustrates the head-to-head stability test of vitamin C concentration retention among gold standard (current benchmark) technology, first technology (ascorbic acid 20, AA20) and second technology (ascorbic acid 15, AA15) as summarized in Table 30 and in Example 11. The results showed that the first technology (AA20) outperformed the gold standard by 500%, taking more than 6× longer to experience the same amount of degradation. The second technology (AA15) containing 15% vitamin C in the formulation outperformed the gold standard by 1000%, taking more than 11× longer to experience the same amount of degradation.

FIG. 9 depicts a microscopy image of a representative subject's (Subject 1) face before and after treatment with the combination of AA20 (formulation 6A of Table 28) and MHC (formulation 1 of Table 1) (also described as “Better Together”) of the present application of Example 13. After treatment with Better Together, a marked improvement in the appearance of wrinkles on forehead, around the eyes, around the mouth, and overall improvement in skin tone evenness was observed at month 2. Reduction of hyperpigmentation on forehead and lower cheeks was also observed. Images were taken with VISIA-CR to control for lighting and positioning.

FIG. 10 depicts a microscopy image of another representative subject's (Subject 2) face before and after treatment with the combination of AA20 (formulation 6A of Table 28) and MHC (Formulation 1 of Table 1) (also described as “Better Together”) of the present application of Example 13. After treatment with Better Together, a marked improvement in the appearance of wrinkles around the eyes, improvement in skin tone evenness, and overall brightening of skin tone was observed at month 1. Reduction of hyperpigmentation on forehead and lower cheeks was also observed. Images were taken with VISIA-CR to control for lighting and positioning.

FIG. 11 Representative case showing marked lightening of periorbital hyperpigmentation above and below the eyes and overall brightening of skin tone at 12 weeks for clinical study 1 of: Ascorbic Acid (AA) 20: Significant Brightening Effects of Example 12.

FIG. 12 shows representative case showing marked brightening of skin on the cheeks and nose, and overall improvement of skin tone at 6 weeks clinical study 1: Ascorbic Acid (AA) 20: Significant Brightening Effects of Example 12.

### DETAILED DESCRIPTION

### Minimalist Hydrating Formulations

Aspects of the present disclosure provides topical formulations of minimalist emulsion compositions. The formulations provide moisturization for dry skin conditions. This disclosure provides particular topical formulations which have been developed and optimized to provide skin compatibility and desirable physical properties.

Topical compositions of this disclosure find use in treating or preventing a variety of cosmetic and/or dermatological conditions. Non-limiting examples of dermatocosmetic conditions that may be improved by topical application of the compositions of the present disclosure include: inflammatory dermatoses (including eczema, acne, psoriasis), and xeroses (also known in the art as dry skin or pruritus).

Traditional emulsions suffer from undesirable physical properties, including perceived heaviness and stickiness or greasiness upon application, especially to the face, uneven or inconsistent texture, as well as perceived irritation and allergenicity in those with sensitive skin. Such emulsions often lack ingredients with significant barrier-repairing properties and often rely on triglycerides and plant-derived oils that break down unpredictably on skin due to differences in microflora present on individuals' skin. The unpredictable breakdown of triglycerides, resulting in free fatty acids, is problematic for a number of reasons, including the potential for flooding skin with free fatty acids that may impair barrier repair of skin, as well as the unpleasant odor that accompanies the release of certain free fatty acids, such as capric and caprylic acids. The inventor of the present disclosure discovered that by incorporating panthenol, alkanediol(s), chelating agent(s), barrier repairing agent(s), non-triglyceride emollient(s), specific emulsifier(s), and soothing agent(s), one can combine the exceeding gentleness with desirable physical properties (viscous texture, perceived lightness on skin, lack of stickiness, reduced or no pilling, pourable texture, shelf-stability).

The inventor of the present disclosure discovered that by limiting the fatty alcohol and triglyceride content to less than 0.5% each, one can limit skin's exposure to free fatty acids which have the potential to delay barrier repair in compromised skin, as well as release an unpleasant odor.

### Minimalist Hydrating Formulation Components

The CTFA International Cosmetic Ingredient Dictionary and Handbook (2014), 15th Edition, describes a wide variety of non-limiting cosmetic ingredients that can be used in the context of the present disclosure. Examples of these ingredient classes include: fragrances (artificial and natural), dyes and color ingredients, adsorbents, emulsifiers, stabilizers, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraaminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., glycerin, propylene glycol, butylene glycol, pentylene glycol, sorbitol, urea, and manitol), exfoliants (e.g., alpha-hydroxyacids, and beta-hydroxyacids such as lactic acid, glycolic acid, and salicylic acid; and salts thereof) waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate), thickening agents (e.g., substances which that can increase the viscosity of a composition such as carboxylic acid polymers, crosslinked polyacrylate polymers, polyacrylamide polymers, polysaccharides, and gums), and silicone containing compounds (e.g., silicone oils and polyorganosiloxanes). The following provides specific non-limiting examples of some of the ingredients that can be used in the formulations of the present disclosure.

The percentages of the ingredients described in the present disclosure include a weight percentage based on the total weight of the final formulation.

i. Panthenol

In certain embodiments, the formulation comprises one or more panthenol compounds. The panthenol compound can function as a moisturizing agent, a barrier repairing agent, or a lubricant in the formulation, without effecting stability of the composition The present inventors found that, in contrast to niacinamide, panthenol did not destabilize the emulsion or effect the optimized pH level within the composition. In some embodiments, the composition does not comprise niacinamide. In some embodiments, the composition does not consist of niacinamide. As used herein, the term “panthenol compound” includes panthenol, one or more pantothenic acid derivatives, and mixtures thereof. Non-limiting examples of panthenol compounds include D-panthenol ([R]-2,4-dihydroxy-N-[3-hydroxypropyl)]-3,3-dimethylbutamide), D,L-panthenol, pantothenic acids and their salts, panthenyl triacetate, royal jelly, panthetine, pantetheine, panthenyl ethyl ether, pangamic acid, pantoyl lactose, Vitamin B complex, or mixtures thereof. Accordingly, the formulation may include from 1% to 15% by weight of the panthenol compound; such as from 3% to 10% by weight of the panthenol compound; from 4% to 8% by weight of the panthenol compound; or from 4% to 6% by weight of the panthenol compound, wherein the percentage is a weight percentage based on the total weight of the final formulation. For example, the formulation may include about 4% by weight of the panthenol compound, about 5% by weight of the panthenol compound, or about 6% by weight of the panthenol compound.

In certain embodiments, the panthenol compound is D-panthenol (also referred to as dexpanthenol). D-panthenol is the alcohol analog of D-pantothenic acid, and has the following structure:

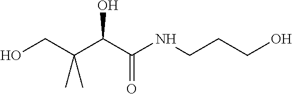

In certain embodiments, the formulation may include from 1% to 15% by weight of D-panthenol; such as from 3% to 10% of D-panthenol; from 4% to 8% by weight of D-panthenol; or from 4% to 6% by weight of D-panthenol, wherein the percentage is a weight percentage based on the total weight of the final formulation. For example, the formulation may include about 4% of D-panthenol, about 5% by weight of D-panthenol, or about 6% of D-panthenol. In certain embodiments, the formulation comprises 5% by weight of D-panthenol. In certain embodiments, the formulation comprises 4% by weight of D-panthenol. In certain embodiments, the formulation comprises 6% by weight of D-panthenol. In certain embodiments, the formulation comprises 1% by weight, 2% by weight, 3% by weight, 4% by weight, 5% by weight, 6% by weight, 7% by weight, 8% by weight, 9% by weight, 10% by weight, 11% by weight, 12% by weight, 13% by weight, 14% by weight, or 15% by weight of D-panthenol. In certain embodiments, the formulation comprises 1% by weight, 2% by weight, 3% by weight, 4% by weight, 5% by weight, 6% by weight, 7% by weight, 8% by weight, 9% by weight, 10% by weight, 11% by weight, 12% by weight, 13% by weight, 14% by weight, or 15% by weight of panthenol. In certain embodiments, the formulation comprises 1% by weight, 2% by weight, 3% by weight, 4% by weight, 5% by weight, 6% by weight, 7% by weight, 8% by weight, 9% by weight, 10% by weight, 11% by weight, 12% by weight, 13% by weight, 14% by weight, or 15% by weight of DL-panthenol.

ii. Alkanediol

In certain embodiments, the formulation comprises one or more alkanediol. The alkanediol can function as a solvent, a viscosity decreasing agent, a fragrance ingredient, a humectant, a skin conditioning agent, an emollient, a preservative and/or a moisturizer in the subject formulation. In certain embodiments, the alkanediol is a (C2-C6)alkane diol, e.g., an ethanediol, a propane diol, a butanediol, a pentanediol or a hexanediol. In certain embodiments, the alkane diol is a (C3-C6)alkane diol, e.g., a propanediol, a butanediol, a pentanediol or a hexanediol. In certain embodiments, the alkanediol is a C3-alkanediol, e.g., a propanediol.

In certain cases, the alkanediol is selected from 1,3-propanediol, propylene glycol, 1,3-butanediol (butylene glycol), 1,4-butanediol, 2,3-butanediol, 1,5-pentanediol, pentylene glycol, 1,2-hexanediol, 1,6-hexanediol, 1,8-octanediol, 1,2-octanediol (caprylyl glycol), 1,10-decanediol, butyl ethyl propane diol and isopentyldiol.

In certain embodiments, the alkanediol is selected from a propanediol, a propylene glycol, a butylene glycol, a pentylene glycol, a hexanediol, and a combination thereof. In certain cases, the alkane diol is 1,3 propanediol. In certain cases, the alkane diol is a combination of 1,2-hexanediol and 1,3-propanediol. In certain cases, the ratio of 1,2-hexanediol to 1,3-propanediol is about 1:22. In certain cases, the ratio of 1,2-hexanediol to 1,3-propanediol is about 1:20. In certain cases, the ratio of 1,2-hexanediol to 1,3-propanediol is about 1:19. In certain cases, the ratio of 1,2-hexanediol to 1,3-propanediol is about 1:23. In certain cases, the ratio of 1,2-hexanediol to 1,3-propanediol is about 1:24. In certain cases, the ratio of 1,2-hexanediol to 1,3-propanediol is about 1:25.

In some embodiments, the formulation may include from 2% to 10% by weight of the alkanediol; such as from 3% to 9% by weight of the alkanediol; from 4% to 8% by weight of the alkanediol; or from 4% to 7% by weight of the alkanediol, wherein the percentage is a weight percentage based on the total weight of the final formulation. For example, the formulation may include about 4% of the alkanediol, about 5% by weight of the alkanediol, about 6% by weight of the alkanediol, or about 7% by weight of the alkanediol. In certain embodiments, the formulation comprises from 6-7% by weight of the alkanediol.

In some embodiments, the formulation comprises from 2% to 10% by weight of 1,3-propanediol; such as from 3% to 9% by weight of 1,3-propanediol; from 4% to 8% by weight of 1,3-propanediol; or from 4% to 7% by weight of 1,3-propanediol, wherein the percentage is a weight percentage based on the total weight of the final formulation. For example, the formulation may include about 4% by weight of 1,3-propanediol, about 5% by weight of 1,3-propanediol, about 6% by weight of 1,3-propanediol, or about 7% by weight of 1,3-propanediol. In certain embodiments, the formulation comprises from 6-7% of 1,3-propanediol. In addition, the formulation may comprise from 0.1 to 0.5% by weight of 1,2-hexanediol; from 0.1 to 0.4% of 1,2-hexandiol; or from 0.2% to 0.4% by weight of 1,2-hexanediol, wherein the percentage is a weight percentage based on the total weight of the final formulation. For example, the formulation may include about 0.2% by weight of 1,2-hexanediol, about 0.3% by weight of 1,2-hexane diol, or about 0.4% by weight of 1,2-hexane diol.

iii. Emulsifier

In certain embodiments, the formulation includes one or more non-ethoxylated emulsifiers. In certain embodiments the emulsifier does not comprise 1,4-dioxane, or an ethoxylated emulsifier. Emulsifiers can reduce the interfacial tension between phases and improve the formulation and stability of the emulsion. The emulsifiers can be synthetic or natural emulsifiers. In certain cases, the emulsifier comprises one or more of a glycolipid, a lipopeptide, a phospholipid, a fatty acid, or a polymeric compound. In certain cases one or more glycolipid is selected from sophorolipids, rhamnolipids and mannosylerythritol lipids.

Non-limiting examples of natural emulsifiers may comprise combinations of cetearyl alcohol and cetearyl glucoside; cetearyl olivate and sorbitan olivate; cetearyl wheat straw glycosides and cetearyl alcohol; or glyceryl stearate alcohol and sodium stearoyl lactylate.

In certain embodiments, the emulsifier comprises a phosphoglyceride. In certain embodiments, the phosphoglyceride is lecithin. In some embodiments, the emulsifier comprises a combination of lecithin and sodium acrylate copolymer, marketed under the tradename Lecigel by Lucus Meyer Cosmetics. In some embodiments, the emulsifier comprises a combination of lecithin and sodium acrylate copolymer in a ratio of 1:3, 1:2.5, 1:2, 1:1.5, or 1:1. In certain embodiments the emulsifier comprises a combination of lecithin and sodium acrylate copolymer in a ratio of 3:1, 2.5:1, 2:1, 1:1.5 or 1:1. In certain embodiments, the emulsifier comprises 10-40% lecithin and 60-90% by weight sodium acrylate copolymer, such as 15-35% by weight lecithin and 65-85% w/w sodium acrylate copolymer, 20-30% w/w lecithin and 70-80% w/w sodium acrylate copolymer, wherein the percentages are weight percentages based on the total weight of the emulsifier. In certain embodiments, the emulsifier comprises about 25% lecithin and about 75% w/w sodium acylate copolymer, wherein the percentages are weight percentages based on the total weight of the emulsifier. In certain embodiments, the emulsifier comprises 25% lecithin and 75% w/w sodium acylate copolymer, wherein the percentages are weight percentages based on the total weight of the emulsifier.

In some embodiments the emulsifier is polyacrylate crosspolymer-6, marketed under the tradename Sepimax Zen by Seppic. In some embodiments, the emulsifier comprises a combination of polyglyceryl-10 mono/dioleate, polyglyceryl-3 oleate, glycerin and phosphatidylglycerol, marked under the tradename Distinctive Emul-Lipid Ba by Vantage. In some embodiments the emulsifier comprises a combination of C14-22 alcohols and C12-20 alkyl glucosides, marketed under the tradename Montanov L by Seppic. The combination of C14-C22 alcohols and C12-C20 alcohols can be obtained from the distilled acetalization product between glucose and C12, C14, C18, C20, C22 alcohols. Any combination of C14-C22 alcohols and C12-C20 alkyl glucosides can find use in the present application. In some embodiments, the emulsifier comprises a combination of C14-22 alcohols and C12-20 alkyl glucosides in a ratio of 1:1, 1:1.5, 1:2, 1:2.5, 1:3, 1:3.5, 1:4, 1:4.5; 1:5, 1:5.5, 1:6, 1:6.5, 1:7, 1.7:5, 1:8, 1:8.5, 1:9, 1:9.5 or 1:10. In certain embodiments, the emulsifier comprises a combination of C14-C22 alcohols and C12-C20 alkyl glucosides in a ratio of 10:1, such as 9.5:1, 9:1, 8.5:1, 8:1, 7.5:1, 7:1, 6.5:1, 6:1, 5.5:1, 5:1, 4.5:1, 4:1, 3.5:1, 3:1, 2.5:1, 2:1, or 1.5:1. In some embodiments, the emulsifier comprises a combination of C10-30 cholesterol/lanosterol esters.

In some embodiments, the formulation may include from 0.5% to 5% by weight of the emulsifier; such as from 0.5% to 4% by weight of the emulsifier; from 0.5% to 3% by weight of the emulsifier; or from 0.5% to 2% by weight of the emulsifier, wherein the percentage is a weight percentage based on the total weight of the final formulation. For example, the formulation may include about 3% of the emulsifier, about 2.5% by weight of the emulsifier, or about 2% by weight of the emulsifier.

In some embodiments, the emulsifier comprises a combination of lecithin and sodium acrylate copolymer in a ratio of 1:3, and the combination is present in an amount from 0.5% to 3%; such as from 0.5% to 2.5%; or from 0.5% to 2%, wherein the percentage is a weight percentage based on the total weight of the final formulation. For example, the formulation may include about 3%, about 2.5%, or about 2% of an emulsifier comprising a combination of lecithin and sodium acrylate copolymer. In some embodiments, the emulsifier comprises a combination of lecithin and sodium acrylate copolymer in a ratio of 1:3, and the combination is present in an amount from 1.6 to 2%, such as 1.6%, 1.7%, 1.8%, 1.9%, or 2%, wherein the percentage is a weight percentage based on the total weight of the final formulation.

iv. Chelating Agent

In certain embodiments, the formulation includes one or more chelating agents. In certain embodiments, the chelating agent is a natural plant derived chelating agent.

In some embodiments the chelating agent includes ethylenediaminetetraacetic acid (EDTA) and salts thereof, e.g., tetrasodium EDTA; nitrilotriacetic acid (NTA) and salts thereof; ethylenediamine disuccinate (EDDS) and salts thereof, e.g., trisodium ethylenediamine disuccinate; citric acid and salts thereof, e.g., sodium citrate; glucono-delta-lactone (GDL); phytic acid; sodium phytate; sodium gluconate; a cyclodexin; and activated charcoal. In certain embodiments, the chelating agent is a phytic acid solution, marketed as dermofeel PA by Evonik. In certain embodiments, the chelating agent is a phytic acid solution in water (50% w/w).

In some embodiments the formulation may include from 0.05% to 0.2% of the chelating agent; such as from 0.05% to 0.1% of the chelating agent; from 0.05% to 0.15% of the chelating agent; or from 0.05% to 0.2% of the chelating agent, wherein the percentage is a weight percentage based on the total weight of the final formulation. For example, the formulation may include about 0.2% of the chelating agent, about 0.15% of the chelating agent, or about 0.1% of the chelating agent. In certain embodiments, the formulation comprises 0.1% of the chelating agent.

v. Emollient

In certain embodiments, the formulation includes one or more emollients. In certain cases emollients can also be referred to as occlusives and humectants. In certain embodiments the emollient is a long chain polyhydric alcoholic ester. In certain cases, the emollient is derived from a vegetable source. In certain embodiments, the emollient is pentaerythrityl tetraethylhexanoate (2,2-bis[[(2-Ethyl-1-oxohexyl)oxy]methyl]propane-1,3-diyl bis(2-ethylhexanoate)).

In certain cases, the emollient does not comprise a triglyceride. In certain embodiments, the emollient is selected from glycerin, sodium lactate, lanolin, beeswax, mineral oil, petrolatum, she butter, safflower oil, stearic acid, linoleic acid, oleic acid, lauric acid and pentaeythrityl tetraethylhexanoate.

In some embodiments the formulation may include from 2% to 20% of the emollient; such as from 2% to 18% by weight of the emollient; from 2% to 16% by weight of the emulsifier; or from 2% to 15% by weight of the emollient, wherein the percentage is a weight percentage based on the total weight of the final formulation. In certain embodiments the formulation may include from 10% to 15% by weight of the emollient; such as from 10% to 14% of the emollient; from 10% to 13% by weight of the emollient; or from 11% to 13% by weight of the emollient, wherein the percentage is a weight percentage based on the total weight of the final formulation. For example, the formulation may include about 14% by weight of the emollient, about 13% by weight of the emollient, or about 12% by weight of the emollient.

vi. Barrier Repairing Agent

In certain embodiments, the formulation includes one or more barrier repairing agent. In certain embodiments the barrier repairing agent comprises a mixture of fatty acids. In certain cases, the fatty acids are derived from lanolin and cholesterol. In some cases, the barrier repairing agent comprises a ceramide. In certain embodiments, the barrier repairing agent comprises C10-30 cholesterol and lanosterol esters. In certain embodiments, the barrier repairing agent comprises a 3:1:1 ratio of ceramides, cholesterol and free fatty acids. In certain embodiments, the barrier repairing agent comprises a 3:0.5:0.5 ratio of ceramides, cholesterol and free fatty acids. In certain embodiments, the barrier repairing agent comprises a 2:0.5:0.5 ratio of ceramides, cholesterol and free fatty acids. In certain embodiments, the barrier repairing agent comprises a 2.5:0.5:0.5 ratio of ceramides, cholesterol and free fatty acids.

In some embodiments, the formulation comprises less than 10% by weight of the barrier repairing agent, such as less than 8% by weight, less than 7% by weight, or less than 6% by weight of the barrier repairing agent. In some embodiments the formulation may include from 2% to 10% by weight of the barrier repairing agent; such as from 2% to 8% by weight of the barrier repairing agent; from 2% to 6% by weight of the barrier repairing agent; or from 2% to 5% by weight of the barrier repairing agent, wherein the percentage is a weight percentage based on the total weight of the final formulation. For example, the formulation may include about 5% by weight of the barrier repairing agent, about 4% by weight of the barrier repairing agent, or about 3% of the barrier repairing agent. In certain embodiments, the formulation comprises 4% by weight of the barrier repairing agent.

vii. Soothing Agent

In certain embodiments, the formulation includes one or more soothing agents. In certain embodiments the soothing agent comprises one or more plant extracts. In certain cases, soothing agent is an extract from Centella asiatica, a perennial plant in the flowering plant family Apiaceae. In certain cases, the soothing agent comprises a triterpenoid saponin compound. In certain cases, the soothing agent also has one or more advantageous properties, such as wound healing properties, anti-inflammatory properties, and antioxidant properties. In some cases, the soothing agent comprises madecassoside, asiaticoside, or a combination thereof. In certain embodiments, the barrier repairing agent comprises madecassoside. In certain cases, the barrier repairing agent comprises a combination of madecassoside and asiaticoside in a ratio of 2.3:1, 2.2:1, 2.1:1, 2:1, 1.7:1, 1:5:1, 1:2:1 or 1:1. In certain cases, the soothing agent comprises an oleanolic acid. In certain cases, the oleanolic acid is glycyrrhetinic acid, and the glycyrrhetinic acid also has anti-inflammatory properties.

In some embodiments, the formulation comprises less than 2% of the soothing agent, such as less than 1.5% by weight, less than 1% by weight, or less than 0.8% by weight of the soothing agent. In some embodiments the formulation may include from 0.1% to 1.5% of the soothing agent; such as from 0.1% to 1.2% by weight of the soothing agent; from 0.1% to 1% by weight of the soothing agent; or from 0.1% to 0.8% by weight of the soothing agent, wherein the percentage is a weight percentage based on the total weight of the final formulation. For example, the formulation may include about 0.2% by weight of the soothing agent, about 0.5% by weight of the soothing agent, or about 0.7% by weight of the soothing agent. In certain embodiments, the formulation comprises 0.2% by weight of the soothing agent. In certain embodiments, the formulation comprises 0.7% by weight of the soothing agent.

viii. Additional Agents

In certain embodiments, the formulation comprises one or more additional agents. In certain cases, the one or more additional agents is a multifunctional agent. In certain cases, the one or more additional agents can function as an emulsifier, a chelating agent, an emollient, a barrier repairing agent, a soothing agent, an antimicrobial agent, an anti-inflammatory agent, a thickening agent, a humectant, a moisturizing agent, or any combination thereof.

In certain embodiments, one or more additional agents comprises a solvent, for example, one or more additional agents may include an additional alkanediol. In certain cases, the additional agent comprises caprylhydroxamic acid (CHA), glycerin, caprylyl glycol, glycyrrhetinic acid, hydoxyethyl acrylate, sodium acryloyldimethyl taurate copolymer, caesalpinia spinosa gum, sodium lactate, or any combination thereof.

The sum of additional agents (e.g., as described herein) may be present in an amount of 10% by weight or less of the composition, such as 9% by weight or less, 8% by weight or less, 7% by weight or less, 6% by weight or less, 5% by weight or less, 4% by weight or less, 3% by weight or less, 2% by weight or less, 1% or less by weight, wherein the percentage is a weight percentage based on the total weight of the final formulation. In some embodiments the total amount of the one or more additional agents (e.g., as described herein) in the formulation is 10% by weight or less, such as 9% by weight or less, 8% by weight or less, 7% by weight or less, 6% by weight or less, 5% by weight or less, 4% by weight or less, 3% by weight or less, 2% by weight or less, 1% by weight or less.

In some embodiments, the composition further includes 10% or less by weight in total of one or more optional additional components selected from an antioxidant, an antimicrobial agent, an anti-inflammatory agent, a skin lightening agent, and a moisturizing agent.

In certain embodiments, one or more additional agents is an antimicrobial agent. In certain cases, the antimicrobial agent is present in an amount of from 0.01 to 1% by weight, such as 0.01 to 0.5% by weight; 0.01 to 0.1% by weight, 0.01 to 0.08% by weight, 0.01 to 0.07% by weight, or 0.01 to 0.06% by weight, wherein the percentage is a weight percentage based on the total weight of the final formulation. In certain cases, the antimicrobial agent is present in an amount of about 0.04% by weight, about 0.05% by weight or about 0.06% by weight, wherein the weight percent is based on the total weight of the final formulation.

In certain embodiments, one or more additional agents is caprylhydroxamic acid (CHA). In some embodiments, the formulation comprises from 0.02 to 1% of CHA by weight, such as from 0.02 to 0.5% by weight of CHA, from 0.02 to 0.2% by weight of the CHA, or 0.02 to 0.06% by weight of the CHA, wherein the percentage is a weight percentage based on the total weight of the final formulation. In certain cases, the CHA is present in an amount of about 0.04% by weight, about 0.05% by weight or about 0.06% by weight, wherein the weight percent is based on the total weight of the final formulation.

In certain embodiments, one or more additional agents is an anti-inflammatory agent. In certain cases, the anti-inflammatory agent is present in an amount of from 0.1 to 1% by weight, such as 0.2 to 0.8% by weight; 0.3 to 0.7% by weight, or 0.3 to 0.6% by weight, wherein the percentage is a weight percentage based on the total weight of the final formulation. In certain cases, the anti-inflammatory agent is present in an amount of about 0.4% by weight, about 0.5% by weight or about 0.6% by weight, wherein the weight percent is based on the total weight of the final formulation.

In certain embodiments, one or more additional agents is glycrrhetinic acid. In some embodiments, the formulation comprises from 0.1 to 1% by weight of glycrrhetinic acid, such as from 0.2 to 0.8% by weight of glycrrhetinic acid, from 0.3 to 0.7% by weight of glycrrhetinic acid, or 0.3 to 0.6% by weight of glycrrhetinic acid, wherein the percentage is a weight percentage based on the total weight of the final formulation. In certain cases, the formulation comprises about 0.4% by weight of glycrrhetinic acid, about 0.5% by weight or glycrrhetinic acid, or about 0.6% by weight glycrrhetinic acid, wherein the percentage is a weight percent based on the total weight of the final formulation.

ix. Minimal or Excluded Components

In certain embodiments, the formulation does not comprise an oil based stabilizer, such as a fatty acid stabilizer. In certain cases, the formulation does not comprise a fatty acid stabilizer selected from cetyl alcohol, cetearyl alcohol, behenyl alcohol, stearyl alcohol, and any combination thereof. In certain cases, the formulation comprises less than 0.5% by weight of an oil based stabilizer (e.g., a fatty acid stabilizer as described herein), such as less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, or even less of an oil based stabilizer, wherein the percentage is a weight percent based on the total weight of the final formulation.

In certain embodiments, the formulation does not comprise a triglyceride. In certain embodiments, the formulation does not consist of a triglyceride. In certain cases, the formulation does not comprise a triglyceride selected from caprylic/capric triglyceride, argan oil, sunflower seed oil, rapeseed oil, olive oil, coconut oil, palm kernel oil, babassu oil, palm oil, apricot kernel oil, tallow, baobab oil, cocoa butter, andiroba seed oil, mango butter, avocado oil, cottonseed oil, marula oil, shea butter, grape seed oil, almond oil, safflower oil, moringa oil, and a combination thereof. In certain cases, the formulation comprises less than 0.5% by weight of triglycerides (e.g., a triglyceride as described herein), such as less than 0.4%, less than 0.3%, less than 0.2% by weight, less than 0.1% by weight, less than 0.09% by weight, less than 0.08% by weight, less than 0.07% by weight, less than 0.06% by weight, less than 0.05% by weight, or even less of triglycerides, wherein the percentage is a weight percent based on the total weight of the final formulation.

In certain embodiments, the formulation does not comprise any ethoxylated emulsifiers. In certain cases, the formulation does not comprise any 1,4-dioxane. In certain cases, the formulation does not comprise any polyethylene glycol (PEG) based emulsifiers. In certain cases, the formulation does not comprise sodium laureth sulfate (SLES), ceteareth, a polyoxyethylene alcohol, a steareth, or a polysorbate. In certain cases, the formulation comprises less than 0.5% by weight of any ethoxylated emulsifiers (e.g., ethoxylated emulsifiers as described herein), such as less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, or even less of ethoxylated emulsifiers, wherein the percentage is a weight percent based on the total weight of the final formulation.

### Containers

Any containers suitable for storing and/or dispensing the subject formulations can be adapted for use. The container can provide a sealed environment for containing the composition, and separation from the atmosphere. The container can prevent during storage undesirable degradation, e.g., from absorption of light and/or moisture from the atmosphere or surrounding environment. Provided are ready-to-use topical preparations of a cream in a multi-use container which is pre-filled with a storage stable composition (e.g., as described herein).

In certain embodiments, the container is a glass container, such as a glass jar. In certain other embodiments, the container is a plastic container. In certain embodiments, the container includes a dispensing means, such as an airless pump.

Additional packaging for the container can be included. In some cases, the packaging provides a further barrier that prevents absorption of light and/or moisture from the atmosphere or surrounding environment.

### Physical Properties

### Viscosity

Aspects of the present disclosure includes emulsion formulations with a viscosity ranging from 10,000 cps to 75,000 cps. The viscosity of the formulations of the present disclosure may change over time.

In some embodiments, the viscosity ranges from 10,000 cps to 75,000 cps. In some embodiments, the viscosity ranges from 15,000 cps to 75,000 cps. In some embodiments, the viscosity ranges from 20,000 cps to 75,000 cps. In some embodiments, the viscosity ranges from 20,000 cps to 50,000 cps. In some embodiments, the viscosity ranges from 15,000 cps to 75,000 cps. In some embodiments, the viscosity ranges from 20,000 cps to 75,000 cps. In some embodiments, the viscosity ranges from 20,000 cps to 50,000 cps. In some embodiments, the viscosity ranges from 40,000 cps to 75,000 cps. In some embodiments, the viscosity ranges from 40,000 cps to 75,000 cps after storage for at least 3 years at room temperature (25° C.). In certain embodiments, the viscosity ranges from 45,000 cps to 75,000 cps at the time of manufacture. In some embodiments, the viscosity ranges from 45,000 cps to 75,000 cps after storage for at least 3 years at room temperature (25° C.). In some embodiments, the viscosity is about 10,000 cps, 15,000 cps, 20,000 cps, 25,000 cps, 30,000 cps, 31,000 cps, 32,000 cps, 33,000 cps, 34,000 cps, 35,000 cps, 36,000 cps, 37,000 cps, 38,000 cps, 39,000 cps, 40,000 cps, 41,000 cps, about 42,000 cps, about 43,000 cps, about 44,000 cps, about 45,000 cps, about 46,000 cps, about 47,000 cps, about 48,000 cps, about 49,000 cps, about 50,000 cps, about 51,000 cps, about 52,000 cps, about 53,000 cps, about 54,000 cps, about 55,000 cps, about 56,000 cps, about 57,000 cps, about 58,000 cps, about 59,000 cps, about 60,000 cps, about 61,000 cps, about 62,000 cps, about 63,000 cps, about 64,000 cps, about 65,000 cps, about 66,000 cps, about 67,000 cps, about 68,000 cps, about 69,000 cps, about 70,000 cps, about 71,000 cps, about 72,000 cps, about 73,000 cps, about 74,000 cps, or about 75,000 cps at the time of manufacture, after storage for at least 1 year at room temperature (25° C.), after storage for at least 1.5 years at room temperature, after storage for at least 2 years at room temperature, or after storage for at least 3 years at room temperature.

pH

Aspects of the present disclosure includes emulsion formulations with a pH ranging from 3.5 to 6.5. In some embodiments, the present disclosure includes emulsion formulations with a pH ranging from 3.5 to 6.0. Aspects of the present disclosure includes emulsion formulations with a pH ranging from 4.0 to 6.0. In some embodiments, the present disclosure includes emulsion formulations with a pH ranging from 5.0 to 6.0. The pH of the formulations of the present disclosure may change over time.

In some embodiments, the pH ranges from 4.5 to 6.0. In some embodiments, the pH ranges from 4.5 to 6.0 after storage for at least 3 years at room temperature (25° C.). In certain embodiments, the pH ranges from 4.5 to 6.0 at the time of manufacture. In some embodiments, the pH ranges from 5.0 to 6.0 after storage for at least 3 years at room temperature (25° C.). In some embodiments, the pH is 3.5, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, or 6.5. at the time of manufacture, after storage for at least 1 year at room temperature (25° C.), after storage for at least 1.5 years at room temperature, after storage for at least 2 years at room temperature, or after storage for at least 3 years at room temperature.

### Pilling

Aspects of the present disclosure include an emulsion formulation with reduce or no pilling. In some embodiments, pilling is classified using a grading scale of 0-3, with 0 being no pilling, 1 being minor or low levels pilling, 2 being a mediocre level of pilling, and 3 being high levels of pilling.

Formulations of the present application include low or zero levels of pilling.

### Cooling Sensation

Formulations of the present disclosure provide advantageous physical properties to the skin of a subject treated with the formulation, such as a cooling sensation of the skin.

### Stickiness

Formulations of the present disclosure provide advantageous physical properties to the skin of a subject treated with the formulation, such as reduced or no levels of stickiness.

### Kit

Another aspect of the present disclosure provides a kit comprising the minimalist hydrating formulation of the present disclosure, and optionally instructions for using the minimalist hydrating formulation. In some embodiments, the kit comprises instructions for using the kit. In some embodiments, the instructions for using the kit comprises: gently applying to the skin (e.g., face) ½ to 1 pump of the composition of any one of claims 1-57, wherein the composition is applied in the morning, in the evening, once daily, twice daily, or as needed. In some embodiments, the composition is applied daily. In some embodiments, the composition is applied twice daily. In some embodiments, the composition is applied as needed.

### Vitamin C Formulation

In another aspect, the present disclosure provides a vitamin C formulation. The vitamin C formulation is a topical formulations of L-ascorbic acid (vitamin C) dissolved in a combination of a urea agent and a non-aqueous skin-compatible solvent. The formulations are storage stable for an extended period of time without significant degradation of the L-ascorbic acid in the composition, are have desirable physical properties. The topical formulations can include high concentrations of the L-ascorbic acid. The topical formulations can include cinnamic acid or derivatives thereof as a penetration enhancer and stabilizing component of ascorbic acid. In some embodiments, the vitamin C formulation is a formulation described in one or both of International Application publication Number WO2020081868; and International Application Number WO2021212075, which applications are incorporated herein by reference in their entireties.

Vitamin C topical compositions of this disclosure find use in treating or preventing a variety of cosmetic and/or dermatological conditions as well as to reduce the appearance of chronological and/or environmentally-caused skin aging, such as facial fine lines and wrinkles, dyschromia or uneven pigmentation, and dark circles under the eyes. Non-limiting examples of dermatocosmetic conditions that may be improved by topical application of the compositions of the present disclosure include: keratoses, melasma, lentigines, liver spots, inflammatory dermatoses (including eczema, acne, psoriasis), and xeroses (also known in the art as dry skin or pruritus). Topical application can be accomplished by use of a biocompatible gel, which may be provided in the form of a patch, or by use of a cream, foam, and the like. Several gels, patches, creams, foams, and the like appropriate for application to wounds.

In some embodiments, vitamin C formulations of the present disclosure include the ingredients: (i) 5 to 28% by weight ascorbic acid; and (ii) urea agent; and (iii) optionally cinnamic acid dissolved in (iv) a non-aqueous skin-compatible solvent.

The present inventor has discovered that the vitamin C formulations of the present disclosure provides the basis for enhancement and acceleration of topical wound healing. Thus, the inventors found that the formulations can be applied topically to facilitate wound healing.

The present inventor has discovered that a urea agent dissolved in a non-aqueous solvent provides for enhanced solubility and penetration of the ascorbic acid in the non-aqueous solvent.

The present inventor has discovered that cinnamic acids or derivatives thereof, such as ferulic acid, in addition to ascorbic acid and urea in a non-aqueous solvent, provide for enhanced stabilizing effects in the formulations of the present disclosure. Thus, incorporation of cinnamic acids and derivatives are penetration enhancers that provide for enhanced stability of ascorbic acid and a urea agent in a non-aqueous formulation.

### Vitamin C Formulation Components

### Ascorbic Acid

This disclosure provides vitamin C formulations that include combination of particular amount of a urea agent in a non-aqueous skin-compatible solvent which together can provide for dissolution of particular amounts of ascorbic acid and which produce skin-compatible liquid compositions in which the ascorbic acid is substantially stable to decomposition. In some embodiments, the amounts of ascorbic acid stably dissolved in the composition are greater than would otherwise be possible without the particular combinations of ingredients provided by the disclosure.

The terms “ascorbic acid”, “L-ascorbic acid” and “vitamin C” are used interchangeably herein, and refer to the naturally occurring vitamin of CAS Registry Number: 50-81-7. Any convenient form of ascorbic acid can be utilized in the subject formulations. In some embodiments, the ascorbic acid used in the high potency Vitamin C concentrate of the present disclosure is a powder.

In particular embodiments, the amount of ascorbic acid in the subject composition is between about 25% by weight and about 28% by weight (e.g., about 25%, about 26%, about 27% or about 28%) where the ratio of ascorbic acid to urea agent (% wt ratio) is 1.0 to 1.3, such as a ratio of 1.25 (i.e., 1.25:1) or a ratio of 1.0 (i.e., 1:1).

In general, the amounts of ascorbic acid in a composition are calculated relative to the solution phase based on the non-aqueous solvent. See e.g., Formulations 1A, 3A, 4A, 6A and 7A of Table 28. However, the amounts of ascorbic acid and other ingredients relative to the emulsion composition as a whole can readily be calculated by the skilled artisan. Formulations 2 and 5 of Table 28 show exemplary emulsion compositions where the % by weight values shown are relative to the total emulsion composition. It is understood that, in some cases, these concentrate solutions having particular amounts of ascorbic acid can be combined with an immiscible ingredient (e.g., an oil component) and an emulsifying agent to produce an emulsion composition (e.g., as described below).

### Urea Agent

The vitamin C formulations of the present disclosure include a urea agent in an amount sufficient to enhance the solubility of ascorbic acid in the non-aqueous skin compatible solvent and to provide a stable solution. The inventor discovered that particular amounts of urea agent can be added to a non-aqueous solvent to provide stable solutions of ascorbic acid at various desired concentration levels. These amounts of urea agent are selected based on observations regarding the maximum amount of ascorbic acid that can be stably dissolved in the particular non-aqueous solvent, and minimum amounts of urea agent that should be included to provide a stable ascorbic acid solution.

Urea agents of interest include, but are not limited to, urea and substituted urea, such as alkyl substituted urea, more particularly mono-substituted or di-substituted alkyl urea (e.g., hydroxyalkyl urea). In some embodiments, the urea agent is a hydroxyalkyl urea, such as hydroxyethyl urea. The urea agent ingredient used in the subject formulations can be a combination of urea and/or substituted urea. For example, the urea agent can be a combination of urea and hydroxyethyl urea. In certain embodiments, the urea agent is urea. In certain embodiments, the urea agent is hydroxyethyl urea.

TABLE 31
Minimum urea agent in 1,3-propanediol compositions
{concentration of AA-12} * 1.25

| ascorbic acid | Minimum urea agent |
|---|---|
| (% wt) | (% wt) |
| 13 | 1.25 |
| 14 | 2.5 |
| 15 | 3.75 |
| 16 | 5 |
| 17 | 6.25 |
| 18 | 7.5 |
| 19 | 8.75 |
| 20 | 10 |
| 21 | 11.25 |
| 22 | 12.5 |
| 23 | 13.75 |
| 24 | 15 |
| 25 | 16.25 |
| 26 | 17.5 |
| 27 | 18.75 |
| 28 | 20 |

For example, for vitamin C compositions including 15% by weight ascorbic acid, at least about 4% urea is included in the 1,3-propanediol solvent. For compositions including 20% by weight ascorbic acid, at least about 10% urea is included in the 1,3-propanediol solvent. For compositions including 25% by weight ascorbic acid, at least about 16% urea is included in the 1,3-propanediol solvent. In some embodiments, additional amounts of urea agent can be included up to a maximum amount of 20% by weight, to provide desirable physical properties, in combination with additional optional minor ingredients.

### Cinnamic Acids and Derivatives Thereof

The vitamin C formulations of the present disclosure can also include a cinnamic acid and sources thereof, which are known to work synergistically with ascorbic acid to provide additional antioxidant protection to skin. Cinnamic acids of interest and sources thereof include, but are not limited to, ferulic acid, caffeic acid and coumaric acid. In some embodiments, the cinnamic acid is ferulic acid. The cinnamic acid ingredient used in the subject formulation can be a combination of ferulic acid and/or substituted cinnamic acids. For example, the cinnamic acid agent used can be a combination of ferulic acid and caffeic acid.

In some embodiments, the subject vitamin C composition includes about 0.1% to 2% by weight of cinnamic acid (e.g., about 0.1%, about 0.5%, about 1%, about 1.5%, or about 2%).

The vitamin C formulations of the present disclosure include cinnamic acid and derivatives thereof (e.g., ferulic acid, caffeic acid, coumaric acid, sinapinic acid, and other phenolic cinnamic acids), cis and trans isomers thereof, salts thereof, equivalents thereof.

### Caffeic Acid and Derivatives Thereof

In some embodiments, the cinnamic acid derivative is a caffeic acid. Caffeic acid is an antioxidant that increases AA's photoprotective effect on skin. It can also stabilize AA in aqueous systems. In some embodiments, the composition of the present disclosure includes caffeic acid or derivatives thereof.

In some embodiments, caffeic acid is readily soluble in non-aqueous solvents. In some embodiments, the non-aqueous solvent is one or more of 1,3 propanediol, 1,2 propanediol, 1,3 butanediol, and dimethyl isosorbide. In some embodiments, isosorbide can increase the effectiveness of caffeic acid by enhancing skin penetration.

In some embodiments, the vitamin C composition of the present disclosure includes 0.1% or more by weight of caffeic acid or derivatives thereof. In some embodiments, the vitamin C composition includes 0.2% or more, 0.3% or more, 0.4% or more, 0.5% or more, 0.6% or more, 0.7% or more, 0.8% or more, 0.9% or more, 1.0% or more, 1.1% or more, 1.2% or more, 1.3% or more, 1.4% or more, 1.5% or more, 1.6% or more, 1.7% or more, 1.8% or more, 1.9% or more, or 2.0% or more by weight of caffeic acid or derivatives thereof. In some embodiments, the vitamin C composition includes about 0.1 to 5.0% by weight of caffeic acid or derivatives thereof (e.g., 0.1% to 0.5%, 0.5% to 1.0%, 1.0% to 1.5%, 1.5% to 2.0%, 2.0% to 2.5%, 2.5% to 3.0%, 3.0% to 3.5%, 3.5 to 4.0%, 4.0 to 4.5%, or 4.5% to 5.0% by weight of caffeic acid or derivatives thereof). In some embodiments, the cinnamic acid derivative is a combination of ferulic acid and caffeic acid. In some embodiments, the cinnamic acid derivative is trans-ferulic acid and caffeic acid.

### Coumaric Acid and Derivatives Thereof

In some embodiments, the cinnamic acid derivative is a coumaric acid. Coumaric acid is an antioxidant that increases AA's photoprotective effect on skin. It can also stabilize AA in aqueous systems. In some embodiments, the composition of the present disclosure includes coumaric acid or derivatives thereof. In some embodiments, coumaric acid comprises p-coumaric acid.

In some embodiments, coumaric acid is readily soluble in non-aqueous solvents. In some embodiments, the non-aqueous solvent is one or more of 1,3 propanediol, 1,2 propanediol, 1,3 butanediol, and dimethyl isosorbide. In some embodiments, isosorbide can increase the effectiveness of coumaric acid by enhancing skin penetration.

In some embodiments, the vitamin C composition of the present disclosure includes 0.1% or more by weight of coumaric acid or derivatives thereof. In some embodiments, the composition includes 0.2% or more, 0.3% or more, 0.4% or more, 0.5% or more, 0.6% or more, 0.7% or more, 0.8% or more, 0.9% or more, 1.0% or more, 1.1% or more, 1.2% or more, 1.3% or more, 1.4% or more, 1.5% or more, 1.6% or more, 1.7% or more, 1.8% or more, 1.9% or more, or 2.0% or more by weight of coumaric acid or derivatives thereof. In some embodiments, the composition includes about 0.1 to 5.0% by weight of coumaric acid or derivatives thereof (e.g., 0.1% to 0.5%, 0.5% to 1.0%, 1.0% to 1.5%, 1.5% to 2.0%, 2.0% to 2.5%, 2.5% to 3.0%, 3.0% to 3.5%, 3.5 to 4.0%, 4.0 to 4.5%, or 4.5% to 5.0% by weight of coumaric acid or derivatives thereof).

### Sinapinic Acid (e.g., Hydroxycinnamic Acids) and Derivatives Thereof

In some embodiments, the cinnamic acid derivative is sinapinic acid or derivatives thereof. Sinapinic acid is an antioxidant that increases AA's photoprotective effect on skin. It can also stabilize AA in aqueous systems. In some embodiments, the composition of the present disclosure includes sinapinic acid or derivatives thereof.

In some embodiments, sinapinic acid or derivatives thereof is readily soluble in non-aqueous solvents. In some embodiments, the non-aqueous solvent is one or more of 1,3 propanediol, 1,2 propanediol, 1,3 butanediol, and dimethyl isosorbide. In some embodiments, isosorbide can increase the effectiveness of sinapinic acid or derivatives thereof by enhancing skin penetration.

In some embodiments, the vitamin C composition of the present disclosure includes 0.1% or more by weight of sinapinic acid or derivatives thereof. In some embodiments, the composition includes 0.2% or more, 0.3% or more, 0.4% or more, 0.5% or more, 0.6% or more, 0.7% or more, 0.8% or more, 0.9% or more, 1.0% or more, 1.1% or more, 1.2% or more, 1.3% or more, 1.4% or more, 1.5% or more, 1.6% or more, 1.7% or more, 1.8% or more, 1.9% or more, or 2.0% or more by weight of sinapinic acid or derivatives thereof. In some embodiments, the composition includes about 0.1 to 5.0% by weight of sinapinic acid or derivatives thereof (e.g., 0.1% to 0.5%, 0.5% to 1.0%, 1.0% to 1.5%, 1.5% to 2.0%, 2.0% to 2.5%, 2.5% to 3.0%, 3.0% to 3.5%, 3.5 to 4.0%, 4.0 to 4.5%, or 4.5% to 5.0% by weight of sinapinic acid or derivatives thereof).

### Skin Compatible Solvent

In addition to the urea agent and optionally cinnamic acid (e.g., as described herein), the high-potency Vitamin C formulations of the present disclosure contain, at least one non-aqueous skin-compatible solvent. A skin compatible solvent is a solvent that does not cause irritation or sensitization when applied topically to the skin. Non-aqueous skin-compatible solvents of interest include polyols, C(2-6) alkanediols, glycol ethers, dimethyl ethers, and combinations thereof.

In some embodiments, the solvent is a skin compatible polyol. A polyol is an organic alcohol solvent having two or more hydroxy groups. In some embodiments, the polyol solvent is a C(3-6)polyol. In some embodiments, the polyol solvent is a polyether polyol. In some embodiments, the polyol solvent is a polyester polyol. Skin compatible polyols of interest include, but are not limited to, glycerol (1,2,3-propanetriol); diglycerol; propylene glycol (1,2-propanediol); dipropylene glycol; 1,3-propanediol; butylene glycol (1,3-butanediol); 1,2-butanediol; pentylene glycol (1,2-pentanediol); 1,5-pentanediol; 1,2-hexanediol; 1,6-hexanediol; 1,2,3-hexanetriol, 1,2,6-hexanetriol; ethoxydiglycol; and dimethyl isosorbide. In some embodiments, the solvent is a glycol ether, a dimethyl ether, or a combination thereof. A preferred skin-compatible solvent is 1,3-propanediol, commercially available from DuPont Tate & Lyle BioProducts LLC under the tradename ZEMEA®. In some embodiments, the solvent is a mixture of 1,3 propanediol and 1,2 hexanediol.

In some embodiments, the vitamin C composition includes about 10 to 99% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more) of a non-aqueous skin compatible solvent. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol and one or more additional skin compatible solvents.

### Additional Components

A vitamin C formulation may contain one or more (optional) additional ingredients. Any convenient ingredient known to the skilled artisan to provide cosmetic/aesthetic benefits can be utilized in the subject formulations. Such cosmetic/aesthetic benefits include, but are not limited to, reducing the appearance of fine lines/wrinkles, improving skin barrier function (by reducing the rate/extent of trans-epidermal water loss), making the skin feel smoother/more supple/softer, creating the appearance of more even skin tone (reducing dyschromia) and/or “glow”/radiance (also described in the art as “brightness”).

In some embodiments, the vitamin C composition further includes one or more optional additional components (e.g., as described herein). In some embodiments, the one or more optional additional components are selected from tocopherols, tocotrienols (e.g., alpha, beta, delta and gamma tocopherols or alpha, beta, delta and gamma tocotrienols), azelaic acid, hydroxy acids (e.g., salicylic acid), panthenol, pinus pinaster bark extract, emulsifying agent, hyaluronic acid complex, madecassoside, madecassoside asiaticoside, acetyl zingerone, bakuchiol, and bis-ethylhexyl hydroxydimethoxy benzylmalonate.

Each optional additional component (e.g., as described herein) may be present in an amount of 10% or less by weight of the composition, such as 9% or less, 8% or less, 7% or less, 6% or less, 5% or less, 4% or less, 3% or less, 2% or less, 1% or less by weight. In some embodiments the total amount of the one or more optional additional components (e.g., as described herein) in the composition 10% or less by weight, such as 9% or less, 8% or less, 7% or less, 6% or less, 5% or less, 4% or less, 3% or less, 2% or less, 1% or less by weight.

In some embodiments, the vitamin C composition further includes 10% or less by weight in total of one or more optional additional components selected from an antioxidant, a skin lightening agent, and a moisturizing agent.

### Tocopherol or Tocotrienol Agent

In some embodiments, the vitamin C composition further includes optional additional component that is a tocopherol or tocotrienol agent. In some embodiments, the tocopherol or tocotrienol agent is a form of Vitamin E selected from alpha, beta, delta and gamma tocopherols and alpha, beta, delta and gamma tocotrienols, and combinations thereof. In some embodiments, the tocopherol or tocotrienol is alpha-tocopherol.

In some embodiments, the tocopherol or tocotrienol agent is present in the composition in an amount of 2% or less by weight, such as 1.5% or less, 1% or less, or 0.5% or less by weight.

In some embodiments of any one of the vitamin C formulations described herein, the formulation excludes tocopherol or tocotrienol agents, e.g., or precursors thereof having vitamin E activity. In certain embodiments of any one of the formulations described herein, the formulation excludes vitamin E acetate.

### Antioxidants

In certain embodiments, the vitamin C formulation contains a secondary antioxidant (i.e., in addition to Vitamin C or the optional additive tocopherol or tocotrienol agent).

In some embodiments, secondary antioxidants include terpenoid antioxidants, and benzoic acid derivatives (e.g., p-hydroxy benzoic acid, gallic acid, or protocatechuic acid).

Pinus Pinaster Bark/Bud Extract (available under the tradename Pycnogenol® from DKSH North America, Inc., or from Res Pharma Industriale under the tradename Pantrofina® Skin360) contains these cinnamic acid derivatives and benzoic acid derivatives, and is, therefore, a preferred secondary antioxidant.

In some embodiments, the secondary antioxidant is zingerone or acetyl zingerone. In some embodiments, the secondary antioxidant is bakuchiol (10309-37-2) a natural terpenoid antioxidant. In some embodiments, the secondary antioxidant is bis-ethylhexyl hydroxydimethoxy benzylmalonate (HDBM).

The secondary antioxidant, when included, is preferably present in an amount in the range of 0.1 to 3%, more preferably 0.1 to 2% by weight of the composition, such as 0.1 to 1% by weight, 0.1 to 0.5% by weight, e.g., about 0.2%, about 0.3%, about 0.4% or about 0.5% by weight. In some embodiments, the secondary antioxidant is acetyl zingerone.

In some embodiments, the antioxidant is suitable for boosting photoprotection from UVA radiation. Additional non-limiting examples of antioxidants include polydatin, phloretin, resveratrol, ferulic acid, and a mixture thereof. In some embodiments, the antioxidant can be combined with one or more UV filters, for example organic UV filters, in a cosmetically acceptable carrier. The UV filter(s) may be UVB filters, UVA filters (UVA1 and/or UVA2 filters), and/or inorganic UV filters (UVA and/or UVB filters).

### Skin Lightening Agents

In certain embodiments, the vitamin C formulation contains a secondary skin lightening agent (e.g., as defined herein) (i.e., in addition to Vitamin C). Skin lightening agents which may be included in compositions of the present disclosure include, but are not limited to: hydroquinone and its derivatives, including, for example, its monomethyl and monobenzyl ethers; licorice root (Glycyrrhiza glabra) extract; azelaic acid; kojic acid; arbutin; retinoids (including all-trans-retinoic acid, adapalene and tazarotene); alpha hydroxy acids, in particular citric acid, lactic acid, and glycolic acid; ellagic acid; gluconic acid; gentisic acid (2,5-dihydrobenzoic acid); 4-hydroxy benzoic acid; salts and esters of the above-mentioned acids, including ammonium lactate and sodium lactate; N-acetyl glucosamine; aloesin, a hydroxymethyl chromone isolated from aloe vera; Vitamin B3 compound or its derivative—niacin, nicotinic acid, niacinamide. Epigallocatechin 3-O-gallate (EGCG), and other catechin constituents of tea extracts, in particular green tea; extract of soybean oil (Glycine soja), including isoflavones; hydroxystilbene; butyl hydroxy anisole; and butyl hydroxy toluene may also be utilized as a skin lightening agent. In some embodiments, the additional skin lightening agent is azelaic acid or arbutin.

### Hydroxy Acids

In some embodiments, the vitamin C formulation contains a hydroxy acid, e.g., a small molecule compound including a carboxylic acid and a hydroxy group. The acid may be an alkyl carboxylic acid or a benzoic acid. The hydroxy group can be a phenol or an alkyl alcohol. In certain embodiments, the hydroxy acid is an alpha-hydroxy carboxylic acid. In certain embodiments the hydroxy acid contains 2-12 carbon atoms, such as 2-6 or 2-4 carbons. Hydroxy acids of interest include, but are not limited to, glycolic acid, lactic acid, mandelic acid, salicylic acid, capryloyl salicylic acid, salicyloyl phytosphingosine, gluconolactone, lactobionic acid, maltobionic acid, and combinations thereof.

### Anti-Inflammatory

In some embodiments, the vitamin C formulation contains an anti-inflammatory agent as an additional ingredient. In some embodiments, the anti-inflammatory agent is madecassoside, madecassoside asiaticoside, or madecassic acid. The anti-inflammatory agent, when included, is preferably present in an amount in the range of 0.1 to 2%, more preferably 0.1 to 1% by weight of the composition, such as 0.1 to 0.5% by weight, or 0.1 to 0.2% by weight. In some embodiments, madecassoside is included in an amount in the range of 0.1 to 0.5%, such as about 0.1% or about 0.2% by weight.

### Exemplary Topical Vitamin C Formulations

In some embodiments, the topical vitamin C composition includes: a) 5% to 28% by weight ascorbic acid; and b) 5% to 20% by weight of a urea agent, wherein the ratio of ascorbic acid to urea agent is between about 1.0 and about 3.5; dissolved in a non-aqueous skin-compatible solvent selected from polyol, C(2-6) alkanediol, glycol ether, dimethyl ether, or a combination thereof. In general, the ascorbic acid is dissolved at a concentration (AA) that is above its maximum concentration in the solvent alone (X), and the urea is dissolved at a concentration that is at least about (AA-X)*1.25. In some embodiments, the urea is dissolved at a concentration that is about (AA-X)*1.25. In some embodiments, the urea is dissolved at a concentration that is (AA-X)*1.25±1% by weight, such as (AA-X)*1.25±0.5% by weight.

In some embodiments, the ratio of ascorbic acid to urea agent in the composition is 1.8 to 2.2. In some embodiments, the topical composition includes about 15% by weight ascorbic acid; about 8% by weight urea agent; a solvent that includes 1,3-propanediol and/or 1,2-hexanediol; and one or more optional additional components. In certain embodiments, the one or more optional additional component includes acetyl zingerone. In certain embodiments, the one or more optional additional component is a tocopherol or tocotrienol (e.g., as described herein).

In some embodiments, the ratio of ascorbic acid to urea agent in the composition is 1.8 to 2.2. In some embodiments, the topical composition includes about 20% by weight ascorbic acid; about 10% by weight urea agent; a solvent that is 1,3-propanediol; and one or more optional additional components.

In some embodiments, the ratio of ascorbic acid to urea agent in the composition is 1.8 to 2.2. In some embodiments, the topical composition includes about 10% by weight ascorbic acid; about 5% by weight urea agent; a solvent that is 1,3-propanediol; and one or more optional additional components. In certain embodiments, the one or more optional additional components include pinus pinaster bark extract. In some embodiments, the composition includes 2% or less by weight of the pinus pinaster bark extract, such as 1.5% or less, 1% or less, or 0.5% or less (e.g., about 0.5% by weight) of the pinus pinaster bark extract.

In some embodiments, the ratio of ascorbic acid to urea agent in the composition is a ratio from 1.0 to 1.3, such as 1.25. In some embodiments, the topical composition includes about 25% by weight ascorbic acid; about 20% by weight urea agent; a solvent that is 1,3-propanediol; and one or more optional additional components. In certain embodiments, the one or more optional additional components include a hydroxy acid, such as glycolic acid, lactic acid, mandelic acid, salicylic acid, capryloyl salicylic acid, salicyloyl phytosphingosine, gluconolactone, lactobionic acid, maltobionic acid, or combinations thereof. In some embodiments, the hydroxy acid is salicylic acid. In some embodiments, the composition includes 3% or less by weight of the hydroxy acid, such as 2% or less, or 1% or less (e.g., about 2% by weight) of the hydroxy acid.

In some embodiments, the ratio of ascorbic acid to urea agent in the composition is about 1 (e.g., 1:1). In some embodiments, the topical composition includes about 5% by weight ascorbic acid; about 5% by weight urea agent; a solvent that is 1,3-propanediol; and one or more optional additional components. In certain embodiments, the one or more optional additional components include panthenol. In some embodiments, the composition includes 10% or less by weight of the panthenol, such as 5% or less, 4% or less, 3% or less, 2% or less, or 1% or less (e.g., about 4% by weight) of panthenol. In some embodiments, the composition includes about 1% to about 6% by weight of the panthenol, such as about 6%, about 5%, about 4%, about 3%, about 2%, or about 1% by weight of panthenol. In certain embodiments, the one or more optional additional components include hyaluronic acid complex. In some embodiments, the composition includes 2% or less by weight of the hyaluronic acid complex, such as 1.5% or less, 1% or less, or 0.5% or less (e.g., about 1% by weight) of the hyaluronic acid complex.

In some embodiments, the vitamin C formulations of the present disclosure are concentrates which are generally: free of silicones, and “substantially free” of water. By “substantially free” of water is meant that (i) water is not intentionally added to the concentrate, and (ii) the amount of water in the concentrate is less than about 2% by weight of the concentrate, preferably less than 1% by weight, more preferably less than about 0.5%, and still more preferably less than about 0.1%. In certain embodiments, the concentrate is also free of oils or lipids.

### Emulsion Compositions

It is understood that any of the non-aqueous liquid compositions having particular amounts of ascorbic acid (e.g., as described herein) can be combined with an immiscible phase or ingredient (e.g., an oil component) to produce a vitamin C emulsion composition. In some embodiments, the non-aqueous liquid composition that makes up the first phase of an emulsion composition is referred to as a concentrate. The liquid concentrate can be mixed with one or more additional components (e.g., an immiscible oil phase or component and an optional emulsifying agent) to produce an emulsion. A variety of methods and ingredients for preparing emulsions are available and can be used in the subject emulsion compositions.

In some embodiments, a vitamin C emulsion composition of this disclosure is referred to as a gel. Any convenient oils and lipids can be utilized in the oil component of the subject emulsions. An oil component or oil phase refers to any phase that is immiscible with the non-aqueous liquid composition. In some embodiments, the oil component is silicone-based, e.g., includes a silicone polymer. In some embodiments, the oil component includes a silicone oil or silicone elastomer, such as a polyorganosiloxane. In some embodiments, the silicone polymers have dual characteristics, and can be used as emulsifiers and/or act as the continuous/dispersed phase of the emulsion composition.

Oils and lipids of interest include, but are not limited to, silicone oils, linseed oil, tsubaki oil, macadamia nut oil, corn oil, mink oil, olive oil, avocado oil, sasanqua oil, castor oil, safflower oil, apricot oil, cinnamon oil, jojoba oil, grape oil, sunflower oil, almond oil, rapeseed oil, sesame oil, wheat germ oil, rice germ oil, rice bran oil, cottonseed oil, soybean oil, peanut oil, teaseed oil, evening primrose oil, eggyoke oil, neetsfoot oil, liver oil, triglycerine, glycerine trioctanate, pentaerythritol tetraoctanate, glycerine triisopalmitate, cholesterol, free fatty acids, and combinations thereof.

Any convenient emulsifying agents or emulsifiers can be utilized in the preparation of the subject vitamin C emulsions to stabilize the composition and prevent separation of the oil component from the solvent solution (e.g., the non-aqueous liquid composition). Exemplary emulsifying agents include but are not limited to polysorbates, laureth-4, potassium cetyl sulfate, and silicone and silicone-elastomer-based emulsifiers and emulsifying blends. In some embodiment, a surfactant such as a monoglyceride, sorbitan fatty acid ester, or polyglycerine fatty acid ester, polyoxyethylene hardened castor oil, polyoxyethylene fatty acid ether, is added thereto in a small amount, and the stability is further improved.

### Storage Stability of the Vitamin C Formulations

High-potency Vitamin C formulations of the present disclosure are capable of maintaining at least 90% of the starting ascorbic acid content when the concentrate is stored at room temperature for 12 months or longer.

In certain embodiments, the vitamin C formulation can be described by the following clauses:

Clause 1. A storage stable topical composition comprising:
a) 5% to 28% by weight ascorbic acid;
b) 5% to 20% by weight of a urea agent;
c) 0.1% to 5% by weight of a cinnamic acid or derivative thereof; and
d) less than 10% by weight in total of one or more optional additional components;
e) dissolved in a non-aqueous skin-compatible solvent comprising polyol, C₍₂₋₆₎ alkanediol, glycol ether, dimethyl ether, or a combination thereof; wherein the ascorbic acid is dissolved at a concentration (AA) that is above its maximum concentration in the solvent alone (X), and the urea is dissolved at a concentration that is at least (AA-X)*1.25.

Clause 2. The composition of clause 1, wherein the composition demonstrates less than 10 mol % degradation of the ascorbic acid after storage for 6 weeks at 40° C.±2° C. in a sealed container.

Clause 3. The composition of clause 1, wherein the composition demonstrates less than 5 mol % degradation of the ascorbic acid after storage for 8 months at 40° C.±2° C. in a multi-use container.

Clause 4. The composition of clause 1, wherein the composition demonstrates less than 10 mol % degradation of the ascorbic acid after storage for 16 months at 40° C.±2° C. in a multi-use container.

Clause 5. The composition of any one of clauses 1-4, wherein the urea agent is urea.

Clause 6. The composition of any one of clauses 1-4, wherein the urea agent is hydroxyethyl urea.

Clause 7. The composition of any one of clauses 1-4, wherein the urea agent comprises a mixture of urea and hydroxyethyl urea.

Clause 8. The composition of any one of clauses 1-7, wherein the solvent is selected from 1,3 propanediol, 1,2 propanediol, 1,3 butanediol, 1,5 pentanediol, 1,2 hexanediol, 1,6 hexanediol, 1,2 hexanediol, glycerol, diglycerol, ethoxydiglycol, dimethyl isosorbide, and a combination thereof.

Clause 9. The composition of clause 8, wherein the solvent is 1,3 propanediol.

Clause 10. The composition of clause 8, wherein the solvent is a mixture of 1,3 propanediol and 1,2 hexanediol.

Clause 11. The composition of any one of clauses 1-10, wherein the one or more optional additional components are selected from tocopherols, tocotrienols (e.g., alpha, beta, delta and gamma tocopherols or alpha, beta, delta and gamma tocotrienols), azelaic acid, hydroxy acids (e.g., salicylic acid), panthenol, pinus pinaster bark extract, emulsifying agent, hyaluronic acid complex, madecassoside, acetyl zingerone, bakuchiol, and bis-ethylhexylhydroxydimethoxybenzylmalonate.

Clause 12. The composition of any one of clauses 1-11, wherein the composition comprises about 5% by weight of ascorbic acid.

Clause 13. The composition of any one of clauses 1-11, wherein the composition comprises about 10% to about 20% by weight of ascorbic acid.

Clause 14. The composition of clause 13, wherein the composition comprises about 10% by weight of ascorbic acid.

Clause 15. The composition of clause 13, wherein the composition comprises about 15% by weight of ascorbic acid.

Clause 16. The composition of clause 13, wherein the composition comprises about 20% by weight of ascorbic acid.

Clause 17. The composition of any one of clauses 1-11, wherein the composition comprises about 25% by weight of ascorbic acid.

Clause 18. The composition of any one of clauses 13-16, wherein the ratio of ascorbic acid to urea agent is 1.8 to 2.2.

Clause 19. The composition of clause 18, wherein the ratio of ascorbic acid to urea agent is 2 to 1.

Clause 20. The composition of any one of clauses 18-19, wherein the optional additional component comprises acetyl zingerone.

Clause 21. The composition of clause 20, wherein the composition comprises 2% or less by weight of the acetyl zingerone.

Clause 22. The composition of clause 21, wherein the composition comprises about 0.5% by weight of the acetyl zingerone.

Clause 23. The composition of any one of clauses 1-19, wherein the cinnamic acid derivative is selected from ferulic acid, caffeic acid, coumaric acid, sinapinic acid, and derivatives thereof.

Clause 24. The composition of any one of clauses 15 and 18-23, wherein the composition comprises:
about 15% by weight ascorbic acid;
about 8% by weight urea agent;
about 1% by weight ferulic acid; and
a solvent that comprises 1,3-propanediol.

Clause 25. The composition of clause 23, wherein the composition comprises 0.1 to 2% by weight of the ferulic acid.

Clause 26. The composition of clause 23, wherein the composition comprises 1% or less by weight of the ferulic acid.

Clause 27. The composition of clause 26, wherein the composition comprises about 0.5% by weight of the ferulic acid.

Clause 28. The composition of clause 1, wherein the composition comprises 60% by weight of the solvent comprising propanediol.

Clause 29. The composition of clause 1, wherein the composition comprises 10% by weight urea.

Clause 30. The composition of clause 1, wherein the composition comprises 0.5% by weight of diglycerin and pinus pinaster bark extract.

Clause 31. The composition of any one of clauses 16 and 23, wherein the composition comprises:
20% by weight ascorbic acid;
10% by weight urea agent
0.5% by weight ferulic acid;
60% by weight propanediol; and
0.5% by weight diglycerin and pinus pinaster bark extract.

Clause 32. The composition of any one of clauses 16 and 23, wherein the composition comprises:
20% by weight ascorbic acid;
10% by weight urea agent
0.5% by weight ferulic acid; and
a solvent that is 1,3-propanediol.

Clause 33. The composition of any one of clauses 15 and 23, wherein the composition comprises:
15% by weight ascorbic acid;
8% by weight urea agent;
0.5% by weight ferulic acid; and
a solvent that is 1,3-propanediol.

Clause 34. The composition of any one of clauses 13-14, wherein the ratio of ascorbic acid to urea agent is between 3 and 3.5.

Clause 35. The composition of any one of clauses 1-29, wherein the optional additional component comprises azelaic acid.

Clause 36. The composition of clause 31, wherein the composition comprises 3% to 10% by weight of the azelaic acid.

Clause 37. The composition of clause 31, wherein the composition comprises about 7.5% by weight of the azelaic acid.

Clause 38. The composition of any one of clauses 14, 23, and 31, wherein the composition comprises:
about 10% by weight ascorbic acid;
about 3% by weight urea agent;
about 0.5% to 2% by weight ferulic acid; and
a solvent that is 1,3-propanediol.

Clause 39. The composition of any one of clauses 1-38, wherein the one or more optional additional components comprises pinus pinaster bark extract.

Clause 40. The composition of clause 39, wherein the composition comprises 2% or less by weight of the pinus pinaster bark extract.

Clause 41. The composition of clause 39, wherein the composition comprises about 0.5% by weight of the pinus pinaster bark extract.

Clause 42. The composition of any one of clauses 1-41, wherein the one or more optional additional components comprises madecassoside (e.g., madecassoside asiaticoside).

Clause 43. The composition of any one of clauses 1-13, wherein the ratio of ascorbic acid to urea agent is a ratio from 1.0 to 1.3.

Clause 44. The composition of any one of clauses 1-13, wherein the ratio of ascorbic acid to urea agent is 1.25 to 1.

Clause 45. The composition of any one of clauses 1-44, wherein the optional additional component comprises a hydroxy acid.

Clause 46. The composition of clause 45, wherein the hydroxy acid is selected from glycolic acid, lactic acid, mandelic acid, salicylic acid, capryloyl salicylic acid, salicyloyl phytosphingosine, gluconolactone, lactobionic acid, maltobionic acid, and combinations thereof.

Clause 47. The composition of clause 45, wherein the hydroxy acid is salicylic acid.

Clause 48. The composition of clause 45, wherein the composition comprises 3% or less by weight of the hydroxy acid.

Clause 49. The composition of clause 45, wherein the composition comprises about 2% by weight of the hydroxy acid.

Clause 50. The composition of clause 17, wherein the composition comprises:
about 25% by weight ascorbic acid;
about 20% by weight urea agent;
about 0.5% to 2% by weight ferulic acid; and
a solvent that is 1,3-propanediol.

Clause 51. The composition of clause 1, wherein the ratio of ascorbic acid to urea agent is 1 to 1.

Clause 52. The composition of any one of clauses 1-23, wherein the optional additional component comprises panthenol.

Clause 53. The composition of clause 52, wherein the composition comprises 10% or less by weight of the panthenol.

Clause 54. The composition of clause 52, wherein the composition comprises about 5% by weight of the panthenol.

Clause 55. The composition of any one of clauses 12 and 51, wherein the composition comprises:
about 5% by weight ascorbic acid;
about 5% by weight urea agent; and
about 0.1 to 2% by weight of ferulic acid;
a solvent that is 1,3-propanediol.

Clause 56. The composition of any one of clauses 51-54, wherein the one or more optional additional components comprises madecassoside (e.g., madecassoside asiaticoside).

Clause 57. The composition of clause 42, wherein the composition comprises about 1% or less by weight of the madecassoside.

Clause 58. An emulsion composition, comprising:
the composition according to any one of clauses 1-57;
an oil component; and
an optional emulsifying agent.

Clause 59. The emulsion composition of clause 58, wherein the oil component is silicone-based.

Clause 60. The emulsion composition of clause 58 or 59, wherein the emulsion composition comprises an emulsifying agent.

Clause 61. The emulsion composition of any one of clauses 58-59, wherein the emulsifying agent is selected from polysorbates, laureth-4, potassium cetyl sulfate and silicone and silicone-elastomer-based emulsifiers and emulsifying blends.

Clause 62. A ready-to-use topical preparation of ascorbic acid in a multi-use container which is pre-filled with a storage stable topical composition according to any one of clauses 1-57, wherein the multi-use container comprises means for dispensing a single dose of the storage stable topical composition.

Clause 63. The preparation of clause 62, wherein the storage stable topical composition demonstrates less than 10 mol % degradation of the ascorbic acid after storage for 6 weeks at 40° C.±2° C. in the container.

Clause 64. The preparation of clause 62, wherein the storage stable topical composition demonstrates less than 10 mol % degradation of the ascorbic acid after storage for 6 months at 25° C.±2° C. in the container.

Clause 65. The preparation of any one of clauses 58-60, wherein the storage stable topical composition is sealed in the container.

Clause 66. The preparation of any one of clauses 58-61, wherein the container is placed in packaging.

Clause 67. A process for stabilizing ascorbic acid for storage, the process comprising:
combining:
1% to 20% by weight urea agent selected from urea, hydroxyethyl urea, and combination thereof,
10% to 94% by weight of a non-aqueous skin-compatible solvent comprising C(3-6)polyol, ethoxydiglycol, dimethyl ether, or a combination thereof,
0.1 to 5% or less of a cinnamic acid or derivatives thereof, and
optionally one or more additional agents; with
5% to 28% by weight ascorbic acid;
thereby dissolving the ascorbic acid to produce storage stable, nonaqueous, single-phase clear liquid composition of ascorbic acid.

Clause 68. The process of clause 67, wherein the one or more additional agents are combined and comprise:
0.5% to 2% pinus pinaster bark extract.

Clause 69. The process of clause 67, wherein the one or more additional agents are combined and comprise:
3% to 10% by weight azelaic acid.

Clause 70. The process of clause 67, further comprising:
combining 0.5% to 2% by weight of acetyl zingerone and 1.5% to 5% by weight of an emulsifying agent to produce a second liquid composition; and
combining the second liquid composition with the liquid composition of ascorbic acid to produce an emulsion.

Clause 71. The process of clause 67, further comprising:
combining 0.5% to 2% by weight of a lipid component and 1.5% to 5% by weight of an emulsifying agent to produce a second liquid composition; and
combining the second liquid composition with the liquid composition of ascorbic acid to produce an emulsion.

Clause 72. The process of clause 71, wherein the lipid component is selected from cholesterol, ceramides, free fatty acids, and combinations thereof.

Clause 73. The process of clause 67, wherein the one or more additional agents are combined and comprise:
0.5% to 2% by weight hydroxy acid.

Clause 74. The process of clause 73, wherein the hydroxy acid is selected from glycolic acid, lactic acid, mandelic acid, salicylic acid, capryloyl salicylic acid, salicyloyl phytosphingosine, gluconolactone, lactobionic acid, maltobionic acid, and combinations thereof.

Clause 75. A product produced by the process according to any one of clauses 67-74.

Clause 76. The product of clause 75, wherein the product is used for wound healing

Clause 77. The product of clause 75, wherein the product is a serum.

### Kits

The present disclosure provides a kit comprising a package containing i) a topical vitamin C formulation (e.g., as described herein); ii) a topical minimalist hydrating formulation (e.g., as described herein; and iii) instructions for use.

In accordance with embodiments of the invention, the kit comprises at least two containers (e.g., as described herein).

In accordance with the invention, the kit comprises instructions (e.g., a leaflet) inserted into the container or box, typically a user information leaflet containing printed information, which information may include a description of the form and composition of the formulations contained in the kit, an indication of the use for which the product is intended, instructions as to how the product is to be used (e.g., with respect to each of the two formulations) and information and warnings concerning adverse effects and contraindications associated with the use. In accordance with the present disclosure, the leaflet will usually contain the information concerning the uses, treatment regimens, etc. as described herein in relation to the methods of use of the present disclosure. In certain cases, the leaflet contains printed instructions to repeatedly (self-)apply the topical formulations in order to reduce skin aging effects such as wrinkling, dark spots, firmness and elasticity.

In some embodiments, the vitamin C formulation comprises:
h. 5% to 28% by weight ascorbic acid;
i. 5% to 20% by weight of a urea agent;
j. 0.1% to 5% by weight of a cinnamic acid or derivative thereof; and
k. less than 10% by weight in total of one or more optional additional components;
l. dissolved in a non-aqueous skin-compatible solvent comprising polyol, C₍₂₋₆₎ alkanediol, glycol ether, dimethyl ether, or a combination thereof; wherein the ascorbic acid is dissolved at a concentration (AA) that is above its maximum concentration in the solvent alone (X), and the urea is dissolved at a concentration that is at least (AA-X)*1.25.

In some embodiments, the minimalist hydrating formulation comprises:
a continuous phase that is at least 75% by weight in total of the composition, comprising:
m. a solvent comprising a mixture of water and an alkanediol; and
n. panthenol;
a dispersed phase that is at least 5% by weight in total, comprising:
o. a non-ethoxylated emulsifier at 0.5% to 3% by weight;
p. an emollient;
q. a barrier repairing agent; and
r. a soothing agent;
with the proviso that the composition comprises:
s. less than 0.5% by weight of oil-based stabilizers (e.g., fatty alcohol stabilizer, e.g., cetyl alcohol) and
t. less than 0.5% by weight of triglycerides; and
u. less than 0.5% by weight of ethoxylated emulsifiers.

In some embodiments, the composition of the minimalist hydrating formulation is storage stable and exhibits a viscosity ranging from 10,000 to 75,000 cps (e.g., 45,000+/−1,000 cps) and pH between 3.5 and 6.5 (e.g., 5.0 to 6.0) when stored at 25° C.

In some embodiments, the minimalist emulsion formulation comprises:
o. 55%-80% by weight of water;
p. 4% to 8% by weight of the alkanediol;
q. 3% to 10% by weight of panthenol;
r. 0.5% to 3% by weight of the non-ethoxylated emulsifier (e.g., lecithin, sodium acrylate copolymer, etc);
s. 2%-18% by weight of the emollient;
t. 2%-10% by weight of the barrier repairing agent;
u. 0.1% to 1.5% by weight of the soothing agent;
with the proviso that the composition does not comprise an oil-based stabilizer or a triglyceride.

In some embodiments, the kit further comprises instructions for using the minimalist hydrating formulation and the vitamin C formulation. In certain embodiments, the instructions for using the minimalist hydrating formulation comprise: applying an effective amount of a topical minimalist hydrating formulation to an area of the skin.

In certain embodiments, the instructions for using the vitamin C formulation comprise applying an effective amount of a vitamin C formulation to the same area of the skin for which the minimalist hydrating formulation was applied.

In certain embodiments, the area of skin is selected from: face, arms, and legs. In certain embodiments, the instructions comprise:
(a) gently applying ½ to 1 pump of the vitamin C formulation to clean skin;
(b) applying the vitamin C formulation in the morning or evening, before or after the use of the minimalist hydrating formulation

In some embodiments, the instructions further comprise using the vitamin C formulation 2-3 times per week, once daily, or initially 2-3 times per week until the skin is acclimated to the vitamin C formulation, followed by once daily.

In some embodiments, the instructions further comprise applying the minimalist hydrating formulation in the morning or evening, once daily or as needed.

In some embodiments, vitamin C formulation is used before applying the minimalist hydrating formulation.

In some embodiments, vitamin C formulation is used after applying the minimalist hydrating formulation. In some embodiments, the vitamin C formulation comprises: Propanediol, Ascorbic Acid, Urea, Ferulic Acid, Diglycerin, and Pinus Pinaster Bark Extract.

In some embodiments, the minimalist hydrating formulation comprises: Water, Pentaerythrityl Tetraethylhexanoate, Propanediol, Panthenol, C10-30 Cholesterol/Lanosterol Esters, Sodium Acrylates Copolymer, Lecithin, Glycyrrhetinic Acid, Madecassoside, Asiaticoside, Phytic acid, Caprylhydroxamic acid, and 1,2-Hexanediol.

In some embodiments, the kit comprises two containers, wherein one container comprises the vitamin C formulation, and a second container comprises the minimalist hydrating formulation.

### Methods

Aspects of the present disclosure include a method of reducing the appearance of skin aging, the method comprising:
i) applying an effective amount of a topical minimalist hydrating formulation of any one of claims 1-59 to an area of skin; and
ii) applying an effective amount of a vitamin C formulation to the same area of skin as the minimalist hydrating formulation;
wherein steps i) and ii) are carried out at least once daily to reduce the appearance of skin aging.

In some embodiments, the vitamin C formulation is applied immediately after or immediately before applying the minimalist hydrating formulation. The method of claim 78, wherein the vitamin C formulation is applied 1 minute after applying the minimalist hydrating formulation, 2 minutes after applying the minimalist hydrating formulation, 3 minutes after applying the minimalist hydrating formulation, 4 minutes after applying the minimalist hydrating formulation, 5 minutes after applying the minimalist hydrating formulation, 6 minutes after applying the minimalist hydrating formulation, 7 minutes after applying the minimalist hydrating formulation, 8 minutes after applying the minimalist hydrating formulation, 9 minutes after applying the minimalist hydrating formulation, or 10 minutes after applying the minimalist hydrating formulation.

In some embodiments, each of the minimalist hydrating formulation and the vitamin C formulation are applied at once daily, at least twice daily, or 2-3 times per week.

Aspects of the present disclosure include methods for using the minimalist hydrating formulation of the present disclosure. Aspects of the present disclosure include methods for using the vitamin C formulation of the present disclosure.

### Definitions

The following definitions are set forth to illustrate and define the meaning and scope of the terms used in the description.

It must be noted that as used herein and in the appended claims, the singular forms “a”, “an”, and “the” include plural referents unless the context clearly dictates otherwise. For example, the term “an emollient” refers to one or more emollients, i.e., a single emollient and multiple emollients. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

“At least one” means one or more, and also includes individual components as well as mixtures/combinations.

Numbers used in describing quantities of ingredients and/or reaction conditions are to be understood as being modified in all instances by the term “about.” Unless otherwise indicated, percentages and ratios are to be understood as based upon the total weight of the concentrate.

Numerical ranges are meant to include numbers within the recited range, and combinations of subranges between the given ranges. For example, a range from 1-5 includes 1, 2, 3, 4 and 5, as well as subranges such as 2-5, 3-5, 2-3, 2-4, 1-4, etc.

The terms “formulation” and “composition” are used interchangeably herein.

As used herein, the term “non-aqueous” refers to compositions that are substantially anhydrous. Non-limiting examples of substantially anhydrous compositions include, for example, 1% or less water in the subject compositions, such as 0.5% or less, 0.4% or less, 0.3% or less, 0.2% or less, or 0.1% or less water.

The term “storage stable” or “shelf stable” refers to a formulation that is stable at room temperature (e.g., 25° C.) for a period of time without any separation of the emulsion. In certain cases, the period of time is 3 months or more, 6 months or more, 1 year or more, 2 years or more, or 3 years or more. In certain cases, the term “storage stable” refers to a formulation that has met industry stability testing requirements, such as freezer/thaw stability testing and accelerated heat stability testing (e.g., as described herein in Example 3).

It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are described herein.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed. All patents and publications referred to herein are expressly incorporated by reference.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### EXAMPLES

### Example 1—Exemplary Formulations of Minimalist Hydration Formulations

The formulations of Tables 1-4 were prepared and assessed as having desirable properties including storage stability (see, e.g., Example 3 below).

TABLE 1
Formulation 1

| | |
|---|---|
| Component | (% by Weight) |

Components of Continuous Phase

| | |
|---|---|
| Water | 69.2% |
| Panthenol compound | 5% dexpanthenol (D-panthenol) |
| Alkanediol | 6.65% 1,3 propanediol and 0.30% |
| | 1,2-hexanediol |
| Chelating Agent | 0.1% phytic acid Solution |
| | (50% (w/w) in water) |

Components of Dispersed phase

| | |
|---|---|
| Emulsifier | 1.5% sodium acrylate copolymer and |
| | 0.5% lecithin |
| Emollient | 12% pentaerythrityl tetraethylhexanoate |
| Barrier repairing agent | 4% C10-30 cholesterol/lanosterol Esters |
| Soothing Agent | 0.14% madecassoside and 0.06% |
| | asiaticoside |

Additional Agents

| | |
|---|---|
| Antimicrobial agent | 0.05% caprylhydroxamic acid |
| Anti-inflammatory agent | 0.5% glycyrrhetinic acid |

TABLE 2
Formulation 2

| | |
|---|---|
| Component | (% by Weight) |

Components of Continuous Phase

| | |
|---|---|
| Water | 70.45% |
| Panthenol compound | 3.75% dexpanthenol (D-panthenol) |
| Alkanediol | 6.65% 1,3 propanediol and 0.30% |
| | 1,2-hexanediol |
| Chelating Agent | 0.1% phytic acid Solution |
| | (50% (w/w) in water) |

Components of Dispersed phase

| | |
|---|---|
| Emulsifier | 1.5% sodium acrylate copolymer and |
| | 0.5% lecithin |
| Emollient | 12% pentaerythrityl tetraethylhexanoate |
| Barrier repairing agent | 4% C10-30 cholesterol/lanosterol Esters |
| Soothing Agent | 0.14% madecassoside and 0.06% |
| | asiaticoside |

Additional Agents

| | |
|---|---|
| Antimicrobial agent | 0.05% caprylhydroxamic acid |
| Anti-inflammatory agent | 0.5% glycyrrhetinic acid |

TABLE 3
Formulation 3

| | |
|---|---|
| Component | (% by Weight) |

Components of Continuous Phase

| | |
|---|---|
| Water | 71.2% |
| Panthenol compound | 5% panthenol |
| Alkanediol | 6.65% 1,3 propanediol and 0.30% |
| | 1,2-hexanediol |
| Chelating Agent | 0.1% phytic acid solution |
| | (50% (w/w) in water) |

Components of Dispersed phase

| | |
|---|---|
| Emulsifier | 1.5% sodium acrylate copolymer and |
| | 0.5% lecithin |
| Emollient | 12% pentaerythrityl tetraethylhexanoate |
| Barrier repairing agent | 2% C10-30 cholesterol/lanosterol Esters |
| Soothing Agent | 0.2% madecassoside |

Additional Agent(s)

| | |
|---|---|
| Antimicrobial agent | 0.05% caprylhydroxamic acid |
| Anti-inflammatory agent | 0.5% glycyrrhetinic acid |

TABLE 4
Formulation 4

| | |
|---|---|
| Component | (% by Weight) |

Components of Continuous Phase

| | |
|---|---|
| Water | 71.2% |
| Panthenol compound | 5% panthenol |
| Alkanediol | 6% 1,3 propanediol, 0.70% caprylyl |
| | glycol |
| Chelating Agent | 0.1% phytic acid solution |
| | (50% (w/w) in water) |

Components of Dispersed phase

| | |
|---|---|
| Emulsifier | 1.5% sodium acrylate copolymer and |
| | 0.5% lecithin |
| Emollient | 12% pentaerythrityl tetraethylhexanoate |
| Barrier repairing agent | 2% C10-30 cholesterol/lanosterol esters |
| Soothing Agent | 0.2% madecassoside |

Additional Agent(s)

| | |
|---|---|
| Antimicrobial agent | 0.15% caprylhydroxamic acid |
| Humectant/moisturizer/ | 0.15% glycerin |
| solvent | |
| Anti-inflammatory agent | 0.5% glycyrrhetinic acid |

TABLE 5
Formulation 5

| | |
|---|---|
| Component | (% by Weight) |

Components of Continuous Phase

| | |
|---|---|
| Water | 76.55% |
| Panthenol compound | 5% dexpanthenol (D-panthenol) |
| Alkanediol | 6.65% 1,3 propanediol and 0.30% |
| | 1,2-hexanediol |
| Chelating Agent | 0.25% phytic acid Solution |
| | (50% (w/w) in water) |

Components of Dispersed phase

| | |
|---|---|
| Emulsifier | .75% sodium acrylate copolymer and |
| | 0.25% lecithin |
| Emollient | 6% pentaerythrityl tetraethylhexanoate |
| Barrier repairing agent | 4% C10-30 cholesterol/lanosterol Esters |
| Soothing Agent | 0.14% madecassoside and 0.06% |
| | asiaticoside |

Additional Agents

| | |
|---|---|
| Antimicrobial agent | 0.05% caprylhydroxamic acid |

TABLE 6
Formulation 6

| | |
|---|---|
| Component | (% by Weight) |

Components of Continuous Phase

| | |
|---|---|
| Water | 81.79% |
| Panthenol compound | 5% dexpanthenol (D-panthenol) |
| Alkanediol | 6.65% 1,3 propanediol and 0.30% |
| | 1,2-hexanediol |
| Chelating Agent | 0.01% phytic acid Solution |
| | (50% (w/w) in water) |

Components of Dispersed phase

| | |
|---|---|
| Emulsifier | .75% sodium acrylate copolymer and |
| | 0.25% lecithin |
| Emollient | 1% pentaerythrityl tetraethylhexanoate |
| Barrier repairing agent | 4% C10-30 cholesterol/lanosterol Esters |
| Soothing Agent | 0.14% madecassoside and 0.06% |
| | asiaticoside |

Additional Agents

| | |
|---|---|
| Antimicrobial agent | 0.05% caprylhydroxamic acid |

TABLE 7
Formulation 7

| | |
|---|---|
| Component | (% by Weight) |

Components of Continuous Phase

| | |
|---|---|
| Water | 69.7% |
| Panthenol compound | 5% panthenol |
| Alkanediol | 6% 1,3 propanediol and 0.70% caprylyl |
| | glycol |
| Chelating Agent | 0.1% phytic acid solution |
| | (50% (w/w) in water) |

Components of Dispersed phase

| | |
|---|---|
| Emulsifier | 1.5% sodium acrylate copolymer and |
| | 0.5% lecithin |
| Emollient | 12% pentaerythrityl tetraethylhexanoate |
| Barrier repairing agent | 2% C10-30 cholesterol/lanosterol esters |
| Soothing Agent | 0.2% madecassoside |

Additional Agent(s)

| | |
|---|---|
| antimicrobial agent | 0.15% caprylhydroxamic acid |
| anti-inflammatory agent | 0.5% glycyrrhetinic acid |

Formulations 1-7 were produced by combining the respective ingredients listed in each of tables 1-7, and adjusting the pH to a range of from 3.5-6.5. Each formulation exhibited a desirable consistency with a soft matte finish. Each of formulations 1-7 exhibit a desirable viscosity at 25° C., was not sticky and exhibited no odor. See Example 3 below for physical properties of Formulation 1. Additionally, the pH of Formulations 1-4 were adjusted to a pH range of 5.0-5.5 using lactic acid and sodium hydroxide, and formed a cream-like consistency with a soft-matte finish.

### Example 2—Comparative Examples of Minimalist Hydration Formulation

Formulations 8-14 in each of Tables 8-14 respectively are comparative examples. Each formulation was evaluated for its physical appearance and properties. Table 15 provides a list of physical properties for each of Formulations 8-14.

TABLE 8
Formulation 8

| | |
|---|---|
| Component | (% by Weight) |

Components of Continuous Phase

| | |
|---|---|
| Water | 76.2% |
| Panthenol compound | 4% panthenol |
| Gelling agent/pseudo- | 1% polyacrylate crosspolymer-6 |
| emulsifier | |
| Chelating Agent | 0.1% phytic acid solution |
| | (50% (w/w) in water) |
| preservative booster | 0.70% caprylyl glycol |

Components of Dispersed phase

| | |
|---|---|
| Emollient | 14% pentaerythrityl tetraethylhexanoate |
| Barrier repairing agent | 1% C10-30 cholesterol/lanosterol esters |
| Soothing Agent | 0.2% madecassoside |

Additional Agent(s)

| | |
|---|---|
| Humectant/moisturizer/ | 1.15% glycerin |
| solvent | |
| Thickening agent | 1% of hydroxyethyl acrylate/sodium |
| | acryloyldimethyl taurate copolymer |
| Gum/thickener | 1% caesalpinia spinosa gum |
| Antimicrobial agent | 0.15% caprylhydroxamic acid |
| Anti-inflammatory agent | 0.5% glycyrrhetinic acid |

Combining the ingredients listed above in Formulation 5 resulted in a viscous formulation having a desirable consistency. However, Formulation 5 exhibited pilling upon repeated application or vigorous rubbing. This may be due to the type and concentration of the thickening agent and the gum/thickener.

TABLE 9
Formulation 9

| | |
|---|---|
| Component | (% by Weight) |

Components of Continuous Phase

| | |
|---|---|
| Water | 71.3% |
| Panthenol compound | 4% panthenol |
| Chelating Agent | 0.1% phytic acid solution |
| | (50% (w/w) in water) |
| Alkanediol | 0.70% caprylyl glycol |

Components of Dispersed phase

| | |
|---|---|
| Emulsifier | 4% blend of C14-22 Alcohols and |
| | C12-20 alkyl glucoside |
| | (obtained from distilled acetalization |
| | product between glucose and C12, |
| | C14, C18, C20, C22 alcohols) |
| Emollient | 14% pentaerythrityl tetraethylhexanoate |
| Barrier repairing agent | 1.5% C10-30 cholesterol/lanosterol |
| | esters |
| Soothing Agent | 0.2% madecassoside |

Additional Agent(s)

| | |
|---|---|
| Humectant/moisturizer/ | 2.15% glycerin |
| solvent | |
| Thickening agent | 0.5% of hydroxyethyl acrylate/sodium |
| | acryloyldimethyl taurate copolymer |
| Gum/thickener | 0.95% caesalpinia spinosa gum |
| Antimicrobial agent | 0.15% caprylhydroxamic acid |
| Anti-inflammatory agent | 0.5% glycyrrhetinic acid |

Combining the ingredients listed above in Formulation 9 resulted in an emulsion with a desirable texture and consistency, such as cream-like properties. However, formulation 9 exhibited slight pilling upon vigorous rubbing.

TABLE 10
Formulation 10

| | |
|---|---|
| Component | (% by Weight) |

Components of Continuous Phase

| | |
|---|---|
| Water | 72.2% |
| Panthenol compound | 4% panthenol |
| Chelating Agent | 0.1% phytic acid solution |
| | (50% (w/w) in water) |
| Alkanediol | 0.70% caprylyl glycol |

Components of Dispersed phase

| | |
|---|---|
| Emulsifier | 3% blend of polyglyceryl-10 mono/dioleate, |
| | polyglyceryl-3 oleate, glycerin and |
| | phosphatidylglycerol |
| | (wherein the blend consists of: 30.0-60.0% |
| | Polyglyceryl-3 Oleate, 30.0-60.1% |
| | Polyglyceryl-10 Mono/Dioleate, 0.50-1.50% |
| | Phosphatidylglycerol, |
| | 3.00-7.00 glycerin) |
| Emollient | 13% pentaerythrityl tetraethylhexanoate |
| Barrier repairing agent | 1.5% C10-30 cholesterol/lanosterol esters |
| Soothing Agent | 0.2% madecassoside |

Additional Agent(s)

| | |
|---|---|
| Humectant/moisturizer/ | 3.15% glycerin |
| solvent | |
| Thickening agent | 1.5% of hydroxyethyl acrylate/sodium |
| | acryloyldimethyl taurate copolymer |
| Antimicrobial agent | 0.15% caprylhydroxamic acid |
| Anti-inflammatory agent | 0.5% glycyrrhetinic acid |

Combining the ingredients listed above in Formulation 10 resulted in a formulation having some desirable physical properties, such as gel-cream-like properties.

TABLE 11
Formulation 11

| | |
|---|---|
| Component | (% by Weight) |

Components of Continuous Phase

| | |
|---|---|
| Water | 73.2% |
| Panthenol compound | 5% panthenol |
| Alkanediol | 0.70% caprylyl glycol |
| Chelating Agent | 0.1% phytic acid solution |
| | (50% (w/w) in water) |

Components of Dispersed phase

| | |
|---|---|
| Emulsifier | 3% blend of polyglyceryl-10 mono/dioleate, |
| | polyglyceryl-3 oleate, glycerin and |
| | phosphatidylglycerol |
| Emollient | 10% pentaerythrityl tetraethylhexanoate |
| Barrier repairing agent | 2% C10-30 cholesterol/lanosterol esters |
| Soothing Agent | 0.2% madecassoside |

Additional Agent(s)

| | |
|---|---|
| Humectant/moisturizer | 3.15% glycerin |
| Thickening agent | 2% of hydroxyethyl acrylate/sodium |
| | acryloyldimethyl taurate copolymer |
| antimicrobial agent | 0.15% caprylhydroxamic acid |
| anti-inflammatory agent | 0.5% glycyrrhetinic acid |

Combining the ingredients listed above in Formulation 11 resulted in a viscous formulation having a cream consistency. Formulation 11 was observed to exhibit some skin irritation.

TABLE 12
Formulation 12

| | |
|---|---|
| Component | (% by Weight) |

Components of Continuous Phase

| | |
|---|---|
| Water | 69.2% |
| Panthenol compound | 5% niacinamide |
| Alkanediol | 6.65% 1,3 propanediol and 0.30% |
| | 1,2-hexanediol |
| Chelating Agent | 0.1% phytic acid Solution |
| | (50% (w/w) in water) |

Components of Dispersed phase

| | |
|---|---|
| Emulsifier | 1.5% sodium acrylate copolymer and |
| | 0.5% lecithin |
| Emollient | 12% pentaerythrityl tetraethylhexanoate |
| Barrier repairing agent | 4% C10-30 cholesterol/lanosterol Esters |
| Soothing Agent | 0.14 % madecassoside and 0.06% |
| | asiaticoside |

Additional Agents

| | |
|---|---|
| Antimicrobial agent | 0.05% caprylhydroxamic acid |
| Anti-inflammatory agent | 0.5% glycyrrhetinic acid |

Combining the ingredients listed above for Formulation 12 resulted in an unstable emulsion. Instead of forming a homogenous texture, an emulsion failed to form, resulting in two separate phases of water and oil. C

TABLE 13
Formulation 13

| | |
|---|---|
| Component | (% by Weight) |

Components of Continuous Phase

| | |
|---|---|
| Water | 70.8% |
| Panthenol compound | 5% dexpanthenol (D-panthenol) |
| Alkanediol | 6.65% 1,3 propanediol and 0.30% |
| | 1,2-hexanediol |
| Chelating Agent | 0.1% phytic acid Solution |
| | (50% (w/w) in water) |

Components of Dispersed phase

| | |
|---|---|
| Emulsifier | .3% sodium acrylate copolymer and |
| | .1% lecithin |
| Emollient | 12% pentaerythrityl tetraethylhexanoate |
| Barrier repairing agent | 4% C10-30 cholesterol/lanosterol Esters |
| Soothing Agent | 0.14% madecassoside and 0.06% |
| | asiaticoside |

Additional Agents

| | |
|---|---|
| Antimicrobial agent | 0.05% caprylhydroxamic acid |
| Anti-inflammatory agent | 0.5% glycyrrhetinic acid |

Combining the ingredients listed above for Formulation 13 resulted in an unstable emulsion. Instead of forming a homogenous texture, an emulsion failed to form, resulting in two separate phases of water and oil.

TABLE 14
Formulation 14
Components of Continuous Phase

| Component | (% by Weight) |
|---|---|
| Water | 66.2% |
| Panthenol | 5% dexpanthenol (D-panthenol) |
| compound | |
| Alkanediol | 6.65% 1,3 propanediol and 0.30% 1,2-hexanediol |
| Chelating Agent | 0.1% phytic acid Solution (50% (w/w) in water) |

Components of Dispersed phase

| | |
|---|---|
| Emulsifier | 3% sodium acrylate copolymer and 1% lecithin |
| Emollient | 12% pentaerythrityl tetraethylhexanoate |
| Barrier repairing | 4% C10-30 cholesterol/lanosterol Esters |
| agent | |
| Soothing Agent | 0.14% madecassoside and 0.06% asiaticoside |

Additional Agents

| | |
|---|---|
| Antimicrobial agent | 0.05% caprylhydroxamic acid |
| Anti-inflammatory | 0.5% glycyrrhetinic acid |
| agent | |

Combining the ingredients listed above for Formulation 14 resulted in an overly viscous texture that pilled on skin upon vigorous rubbing.

A compilation of physical properties for each of Formulation 1 and 3-9 is provided as Table 15, below:

TABLE 15
Physical properties of Formulations (Form.) 1-14

| Physical property | Form. 1 | Form. 2 | Form. 3 | Form. 4 | Form. 5 | Form. 6 | Form. 7 | Form. 8 |
|---|---|---|---|---|---|---|---|---|
| Pilling (e.g., | 0 | 0 | 0 | 0 | 0 | 0 | 3 | 1 |
| result of film | | | | | | | | |
| formation) | | | | | | | | |
| 0 = no pilling | | | | | | | | |
| 1 = mild pilling | | | | | | | | |
| 2 = moderate pilling | | | | | | | | |
| 3 = high pilling | | | | | | | | |
| Viscosity (CPS) | ≥45,000 | ≥45,000 | ≥45,000 | ≥45,000 | ≥10,000 | ≥10,000 | ≥45,000 | ≥45,000 |
| Cooling sensation | Yes | Yes | Yes | Yes | Yes | Yes | No | Yes |
| Stickiness | No | No | No | No | No | No | No | No |
| Greasiness | No | No | No | No | No | No | No | No |
| Texture (thin | Cream | Cream | Cream | Cream | Lotion | Lotion | Cream | Gel- |
| liquid/lotion/Gel/ | | | | | | | | Cream |
| Gel-cream/cream) | | | | | | | | |
| pH | 5-5.5 | 5-5.5 | 5-5.5 | 5-5.5 | 3.5-4.0 | 6-6.5 | 5-5.5 | 5-5.5 |

| Physical property | Form. 9 | Form. 10 | Form. 11 | Form. 12 | Form. 13 | Form. 14 |
|---|---|---|---|---|---|---|
| Pilling (e.g., | 1 | 1 | 3 | 0 | 0 | 3 |
| result of film | | | | | | |
| formation) | | | | | | |
| 0 = no pilling | | | | | | |
| 1 = mild pilling | | | | | | |
| 2 = moderate pilling | | | | | | |
| 3 = high pilling | | | | | | |
| Viscosity (CPS) | ≥45,000 | ≥30,000 | ≤45,000 | ≤5,000 | ≤5,000 | ≥45,000 |
| Cooling sensation | No | No | No | No | No | Yes |
| Stickiness | Yes | No | Yes | No | No | Yes |
| Greasiness | No | No | No | No | No | No |
| Texture (thin | Cream | Gel- | Cream | Thin, non- | Thin, non- | Too Thick, |
| liquid/lotion/Gel/ | | cream | | homogenous | homogenous | Creamy |
| Gel-cream/cream) | | | | liquid | liquid | |
| pH | 5-5.5 | 5-5.5 | 5-5.5 | 5-5.5 | 5-5.5 | 5-5.5 |

Based on the comparative studies as shown in Example 2, the present inventor surprisingly and unexpectedly found that if the percentages of the components of Formulation 1 of the minimalist hydrating formulation were changed (e.g., decreased/increased) or eliminated, the following undesirable properties to the emulsion formulation would occur:
if the emulsifier containing a blend of lecithin and sodium acrylate copolymer (e.g., 1:3 lecithin: sodium acrylate copolymer) is increased above 2% by weight, the formulation would exhibit pilling;
if the alkanediol 1,3 propanediol is decreased below 6.65% by weight, the formulation would fail to pass the preservative/antimicrobial efficacy testing during manufacture;
if the alkanediol 1,3 propanediol is increased above 6.65% by weight, the formulation would eliminate the “cooling” sensation of the emulsion; and it would increase the stickiness texture of the formulation significantly, affecting the sensorial profile in a negative direction;
If the panthenol (e.g., 5% dexpanthenol (D-panthenol)) is decreased below 1%, the formulation would fail to demonstrate the same level of barrier function improvement as Formulation 1 containing 5% Panthenol;
If the barrier repairing agent containing cholesterol/lanosterol esters were decreased below 2%, the formulation would fail to demonstrate the same amount of barrier function improvement
If the barrier repairing agent containing cholesterol/lanosterol esters were increased above 10%, the formulation would result in a greasy/shiny texture, affecting the sensorial profile in a negative direction;
if the emulsifier containing lecithin and sodium acrylates copolymer (e.g., 1:3 lecithin: sodium acrylate copolymer) is decreased below 1.6%, the emulsion would be unstable, e.g., separation of the oil and water phases;
If the chelating agent (e.g., phytic acid (50% w/w in water)) was decreased or eliminated, the pH of the emulsion would rise beyond the 3.5-6.5 range that is desired; and
If the chelating agent (e.g., phytic acid (50% w/w in water)) increased beyond 0.2%, the pH would drop below the 3.5-6.5 range that is desired.

### Example 3—Physical Properties and Stability Data for Formulation 1 of Minimalist Hydration Formulation

Table 16 illustrates the physical properties for Formulation 1.

TABLE 16
Physical properties of Formulation 1 at 25° C.

| Property | Observation |
|---|---|
| Color | Off-white |
| Odor | Characteristic |
| Appearance | Opaque, viscous lotion |
| pH | 5.20 |
| Viscosity | 55,000 cps |
| Specific Gravity (Sp. Gr.) | 1.00 |
| % Solids | 23.92% |

Two samples of Formulation 1 were packaged and submitted to accelerated stability testing to determine if the formulation would remain stable under various temperature changes. Formulation 1-A was packaged in a glass jar, and Formulation 1-B was packaged in a plastic jar with airless pump. Two stability testing methods were performed on each of the formulations 1-A and 1-B:
i) Freeze/thaw testing; and
ii) Accelerated heat testing.

The results are shown in Tables 17-20 below. Tables 17 and 18 demonstrate that both Formulations 1-A and 1-B are stable after 3 freeze/thaw cycles. Tables 19 and 20 further demonstrate that both formulations 1-A and 1-B are stable for at least three years at room temperature (25° C.). Note that for the storage stability study results of Tables 19-20, three months at 45° C. represents and simulates about 3 years of shelf life at room temperature. One month at 45° C. represents about 1 year of shelf life at room temperature. 12 weeks at 40° C. represents about 2 years of shelf life at room temperature. 12 weeks at 45° C. represents about 3 years of shelf life at room temperature.

TABLE 17
Freeze/thaw testing of Formulation 1-A

| | Cycle Number | | |
|---|---|---|---|
| Property | 1 | 2 | 3 |
| Color | Conforms | Conforms | conforms |
| Odor | Conforms | Conforms | Conforms |
| Appearance | Conforms | Conforms | Conforms |
| pH | 5.69 | 5.35 | 5.59 |
| Viscosity (cps) | 57,000 | 66,000 | 62,000 |

*“Conforms” as used in Table 17 is defined as conforming to the physical property of Formulation 1 as outlined in Table 16.

Acceptable pH ranges at the time of manufacture include a pH range of 3.5-6.5 which is shown to be beneficial to skin and storage-stable.

Acceptable viscosity ranges at the time of manufacture include a viscosity range of 15,000-75,000 cps.

TABLE 18
Freeze/thaw testing of Formulation 1-B

| | Cycle Number | | |
|---|---|---|---|
| Property | 1 | 2 | 3 |
| Color | Conforms | Conforms | conforms |
| Odor | Conforms | Conforms | Conforms |
| Appearance | Conforms | Conforms | Conforms |
| pH | 5.28 | 5.59 | 5.74 |
| Viscosity | 59,000 | 64,000 | 58,000 |

*“Conforms” as used in Table 18 is defined as conforming to the physical property of formulation 1 as outlined in Table 16.

Acceptable pH ranges at the time of manufacture include a pH range of 3.5-6.5 which is shown to be beneficial to skin.

Acceptable viscosity ranges at the time of manufacture include a viscosity range of 10,000-75,000 cps.

TABLE 19
Accelerated Heat Testing of Formulation 1-A

| | | 1 Week | 1 month | 2 months | 3 months |
|---|---|---|---|---|---|
| 5° C. | Color | Conforms | Conforms | Conforms | Conforms |
| | Odor | Conforms | Conforms | Conforms | Conforms |
| | Appearance | Conforms | Conforms | Conforms | Conforms |
| | pH | 5.39 | 5.62 | 5.55 | 5.60 |
| | Viscosity | 49,500 | 52,500 | 51,500 | 49,000 |
| 25° C. (RT) | Color | Conforms | Conforms | Conforms | Conforms |
| | Odor | Conforms | Conforms | Conforms | Conforms |
| | Appearance | Conforms | Conforms | Conforms | Conforms |
| | pH | 5.69 | 5.75 | 5.64 | 5.62 |
| | Viscosity | 54,500 | 52,000 | 52,000 | 49,000 |
| 40° C. w/75% | Color | Conforms | Conforms | Conforms | Conforms |
| RH | Odor | Conforms | Conforms | Conforms | Conforms |
| | Appearance | Conforms | Conforms | Conforms | Conforms |
| | pH | 5.79 | 5.82 | 6.03 | 5.90 |
| | Viscosity | 51,500 | 48,500 | 43,500 | 38,500 |
| 45° C. | Color | Conforms | Slightly darker | Slightly darker | Slightly darker |
| | Odor | Conforms | Conforms | Conforms | Conforms |
| | Appearance | Conforms | Conforms | Conforms | Conforms |
| | pH | 5.92 | 5.79 | 5.95 | 6.01 |
| | Viscosity | 51,500 | 46,500 | 38,500 | 35,500 |

*“Conforms” as used in Table 19 is defined as conforming to the physical property of formulation 1 as outlined in Table 16.

Acceptable pH ranges at the time of manufacture include a pH range of 4.5-6.0 which is shown to be beneficial to skin.

Acceptable viscosity ranges at the time of manufacture include a viscosity range of 10,000-75,000 cps.

The results of Table 19 show that formulation 1 is shelf stable (e.g., storage stable) over 3 years without any separation of the emulsion.

TABLE 20
Accelerated Heat Testing of Formulation 1-B

| | | 1 Week | 1 month | 2 months | 3 months |
|---|---|---|---|---|---|
| 5° C. | Color | Conforms | Conforms | Conforms | Conforms |
| | Odor | Conforms | Conforms | Conforms | Conforms |
| | Appearance | Conforms | Conforms | Conforms | Conforms |
| | pH | 5.90 | 5.60 | 5.58 | 5.60 |
| | Viscosity | 50,000 | 48,000 | 51,500 | 50,000 |
| 25° C. (RT) | Color | Conforms | Conforms | Conforms | Conforms |
| | Odor | Conforms | Conforms | Conforms | Conforms |
| | Appearance | Conforms | Conforms | Conforms | Conforms |
| | pH | 5.76 | 5.41 | 5.61 | 5.68 |
| | Viscosity | 50,000 | 52,000 | 52,500 | 50,000 |
| 40° C. w/75% | Color | Conforms | Conforms | Conforms | Conforms |
| RH | Odor | Conforms | Conforms | Conforms | Conforms |
| | Appearance | Conforms | Conforms | Conforms | Conforms |
| | pH | 5.74 | 5.63 | 5.96 | 5.96 |
| | Viscosity | 47,500 | 45,500 | 42,500 | 38,000 |
| 45° C. | Color | Conforms | Slightly darker | Slightly darker | Slightly darker |
| | Odor | Conforms | Conforms | Conforms | Conforms |
| | Appearance | Conforms | Conforms | Conforms | Conforms |
| | pH | 5.75 | 5.82 | 5.96 | 6.04 |
| | Viscosity | 48,500 | 44,000 | 40,000 | 35,000 |

*“Conforms” as used in Table 20 is defined as conforming to the physical property of formulation 1 as outlined in Table 16.

Acceptable pH ranges at the time of manufacture include a pH range of 4.5-6.0 which is shown to be beneficial to skin.

Acceptable viscosity ranges at the time of manufacture include a viscosity range of 10,000-75,000 cps.

The results of Table 20 show that formulation 1 is shelf stable (e.g., storage stable) over 3 years without any separation of the emulsion.

### Example 4: Trial of Formulation 1 of Minimalist Hydration Formulation on Subjects with Sensitive Skin

To investigate the absence of the skin primary and cumulative irritation potential and skin sensitization potential of formulation 1, subjects with sensitive skin were treated with a controlled product amount to a particular application site over a 6-week period and monitored by a dermatologist.

### Methodology

In this study, the patch test methodology of Kligman et al. was used (KLIGMAN, A. M. & WOODING, W. M. A method for the measurement and evaluation of irritants of human skin. J. Invest. Derm. 49: 78-94, 1967.). This methodology is also known as a contact test or

### Epicutaneous Test.

Both formulation 1 (0.05 g/cm2, also referred herein below as “the product”) and a control (sterile saline solution (NaCl 0.9%)) were applied to patch test filter paper discs, then attached to the right or left back (scapular area) of the study subjects. The two patches were applied to the same dorsum area of the subjects throughout the induction period of the study. The study was initiated with 82 subjects:
69 of the subjects were female
13 of the subjects were male
Age range of subjects from 18 to 58 years old, with a mean age of 35 years old.

Induction Period (3 weeks): The product was always applied to the same duly protected area
(right or left scapular area of the subjects). The applications were made three times a week for three consecutive weeks and the product remained in contact with the skin for 48 hours during the week and for 72 hours during weekends.

Rest Period (10 days): There was a rest period of, at least, 10 days following the induction period, during which neither the product nor the control were applied.

Challenge period: After the rest period, the product and control were applied to the right or
left back of the subjects on a virgin area, that is, where no products had been applied before.

The product was removed by the trained technicians after approximately 48 hours of contact with the subjects' skin. The assessments (readings) were performed immediately (48 h reading) and 24 hours (72 h reading) after the product removal.

Subjects were instructed to contact the study coordinator at any time, in case they presented any complaints. If so, they would be sent for evaluation and guidance by the dermatologist in charge, who would evaluate the subjects, then rate the reaction and follow the appropriate procedure (guidance and/or medication and photographic record, when necessary).

The readings (48 hours after application) were performed soon after the removal of the test. product, in all cases in which no clinical signs were observed. If it were observed, the readings would be performed after, at least, 30 minutes and, at most, 60 minutes so that the signs, possibly caused by the removal of the test product, did not represent a false positive result.

During the study, the areas of product and control applications were evaluated and in case of any clinical sign, it was classified according to a scale recommended by the International Contact Dermatitis Research Group—ICDRG—(FISHER, 1995), show below in Table 21.

TABLE 21
Scale published by the ICDRG

| Reaction | Result |
|---|---|
| 0 − None | Negative (−) |
| 1 − Mild Erythema | Doubtful (?) |
| 2 − Clear Erythema | Positive (+) |
| 3 − Erythema + Edema + Papules | Positive (++) |
| 4 − Erythema + Edema + Papules + Vesicles | Positive (+++) |

Results: Of the 82 subjects who initiated the study:
62 subjects completed the study;
19 subjects were absent due to personal reasons unrelated to the study
1 subject was removed from the study due to an adverse event not related to the product. Namely, irritation caused by continuous exposure of the subjects skin to the adhesive tape used to apply the patch.

Over the course of the study:
No subjects presented any clinical signs at the product (formulation 1 of minimalist hydration formulation) application site; and
No subjects presented any clinical signs at the control site.

Table 22 summarizes the clinical responses observed during the study according to the scale published by the ICDRG as outlined in Table 21.

TABLE 22

| Number of | Positive responses | Positive response |
|---|---|---|
| subjects who | during the | during the |
| completed the study | induction period | Challenge Period |
| 62 | 0 | 0 |
| | Description | Number of sensitization |
| | of the signs | confirmed responses |
| | N/A | 0 |

More specifically, no clinical signs were observed during either the induction period or the challenge period:

Induction period: No reactions were observed during the induction period. The results for this phase indicate the absence of irritation potential for the product.

Challenge period: No reactions were observed during the challenge phase. The results for this phase indicate the absence of allergenic potential for the product.

### Conclusion

According to the methodology used to assess the absence of the skin primary and cumulative irritation potential and skin sensitization of formulation 1 (“the product”) it was concluded that:
The product did not induce a skin primary and cumulative irritation process in the study group.
The product did not induce a skin sensitization process in the study group.
The product was considered safe under the study conditions.

As such, Formulation 1 of minimalist hydration formulation has been dermatologically tested and found to be suitable for use on sensitive skin and storage and shelf-stable.

### Example 5—Clinical Trial of Formulation 1 of Minimalist Hydration Formulation on Subjects to Assess Physical Properties of Skin

To investigate physical changes in skin on subjects in Brazil, subjects were treated with a controlled product amount to a particular application site over a 14-day period and monitored by a dermatologist. An aim of this study was to test the minimalist hydration formulation's ability to ameliorate various signs of transient and chronic skin dryness. Clinical grading, self-assessment, and quantitative measurements were carried out after 14 days of treatment with Formulation 1. A brief summary of the protocol is provided below:

31 patients, all female with an average age of 42 (ranging from 18-59), were included in this study.

Patients were instructed to apply product twice daily at home, under normal conditions of use, for 14+2 days. Patients were instructed to record in a daily log of product use all the applications performed and possible comments about the product.

After 14+2 days of product use, subjects returned to the institute and clinical efficacy assessments were performed by a visual/tactile clinician grading, instrumental measurements with a Corneometer CM 825 device, and image acquisition of the skin with a Visioscan VC20plus device.

Patients were also asked to answer a self-assessment questionnaire

### Materials and Methods

### Subject Inclusion Criteria and Restrictions

A total of 34 female subjects were enrolled and a total of 31 completed the study. Subjects must have no pathologies that could interfere with the study results. Subjects were aged between 18 and 59 years old (mean age: 42 years old), presenting facial dull skin, facial dry skin, visible dryness on lower leg and dry skin on the lower leg (corneometry value of 45 a.u. as maximum, in the initial measurement). Subjects must not change any cosmetic and/or hygienic habit during the trial.

### Withdrawal and Adverse Events

Subjects may have early withdrawn if, for example, the subjects present complications affecting their suitability between the signature of the ICF and the beginning of the study, subjects who lack of adhesion to the study, subjects who have serious adverse events, or subjects who have concurrent disorder or treatment such as any pathological process or treatment that occurred during the study period and that might interfere with the study product, such as a medication interaction or masking of results.

An adverse event is any untoward medical occurrence in a patient or clinical investigation subject administered a product and that does not necessarily have a causal relationship with this treatment. An adverse event can therefore be any unfavorable and unintended sign (including an abnormal laboratory finding), symptom, or disease temporally associated with the use of the investigational product (adapted from ICH, 2016).

According to the Good Clinical Practices (ICH, 2016), a Serious Adverse Event is any untoward medical occurrence that at any dose:
results in death;
is life-threatening;
requires inpatient hospitalization or prolongation of existing hospitalization;
results in persistent or significant disability/incapacity;
is a congenital anomaly/birth defect

Thus, any new sign, symptom or disease, or clinically significant worsening compared to the condition at the first visit, should be considered an Adverse Event. Lack of clinical or self-assessment efficacy of a cosmetic product or drug is not considered an Adverse Event. Clinical signs and dermatological or systemic diseases observed during the selection process of the study subjects are not considered as Adverse Events. The adverse events occurred as a result of incorrect product use (either cosmetics or drugs products)—such as inappropriate frequency or incorrect application—are considered as adverse events that do not interfere with the product evaluation.

### Methodology

On the initial visit (TO) the subjects were informed about the study objective, its methodology and duration, also about the possibly expected benefits and constraints related to the study and they signed an Informed Consent Form (ICF) and an Informed Consent for Image Release (ICIR). The study subject was evaluated by a clinic technician to confirm the inclusion and non-inclusion criteria. To confirm the inclusion criteria of presenting corneometry value of 45 a.u. as maximum on lower legs where instrumental measurements using the device, e.g., Corneometer® CM 825, were performed. These first measurements were considered the time-point baseline in data analysis for the subjects approved. Clinical Efficacy Assessments also was carried out by an expert grader in the approved subjects and images acquisition of the lower legs with the device, e.g., Visioscan® VC 20plus, was done by a technician.

Subjects received the product for use at home, under normal conditions of use, for 14±2 days and the product daily log. Subjects were instructed to apply the product on face and lower legs twice a day. During this period, subjects were instructed to record in the daily log of the product use all the applications performed and possible comments about the product.

After 14±2 days of product use (T14), subjects returned to the institute and Clinical Efficacy Assessments were performed by an expert grader, instrumental measurements with the device Corneometer® CM 825 and images acquisition of the lower legs with the device Visioscan® VC 20plus was done by a technician. The subjects also were asked to answer a self-assessment questionnaire.

At all visits, subjects remained at rest in a room with controlled temperature and humidity (20° C.±2° C. and 50%±5 RH) for at least 15 minutes before the assessments and during the measurements.

### Expert Grading Assessments

Visual and tactile assessments were performed by the expert grader using 10-point scale on the face and lower leg of subjects using overhead lighting as well as a lighted magnifying loop as needed. Natural sunlight was blocked from the room to ensure the same lighting conditions at each time point.

All efforts were made to ensure that the same expert grader assesses the same subjects at every time point. Table 23 summarizes the attributes being evaluated. The evaluations were performed at two time-points: T0=before investigational product use; and T14=after 14 days of investigational product use.

TABLE 23
Expert grading assessment attributes and scales

| | Area | Type of | |
|---|---|---|---|
| Attribute | Assessed | grading | Scale |
| Skin | Face | Visual | 0-Very hydrated skin |
| Hydration | | | 9-Very dry skin |
| Skin Softness | Face | Tactile | 0-Smooth, soft surface feel |
| | | | 9-Rough, coarse surface feel |
| Skin | Face | Visual | 0-Radiant, Luminous, Glowing |
| Radiance | | | appearance |
| | | | 9-Dull/Matte and/or Sallow |
| Skin Texture | Lower leg | Visual | 0-Very smooth and regular texture |
| | | | 9-Extremely rough and uneven texture |

### Corneometer CM 825 Measurements

The moisturization measurements were performed by using the device Corneometer® CM 825 (Courage+Khazaka electronic GmbH), through a measuring probe. The readings were performed with the application of the probe to the test site with the pressure allowed by the spring (3.5 N). Four measurements were performed in each area. The reading indicates the degree of humidity on the skin surface based on variations in electric capacity. The apparatus scale is arbitrary, i.e., greater reading values indicate greater moisturization. The measurements were done to the same site or in a close vicinity.

The measurements were performed on the left or right lower leg (previously randomized) (APPENDIX 4) at the following time-points: T0=before investigational product use; and T14=after 14 days of investigational product use.

### Visioscan VC 20Plus Measurements

The video dermatoscope Visioscan® VC 20plus is a device that allows taking pictures of the skin through magnifying lenses. The images enable a view of the skin topography, texture, dryness, desquamation, blemishes, pores and cuticles.

The dryness aspect was evaluated using the SELS system (Surface Evaluation of the Living Surface), which is based on the graphic representation of the living skin under special lighting, with electronic processing to assess the image (MELO & CAMPOS, 2016).

The resulting images show the skin structure, its dryness degree and the distribution of the gray scale. In this study, the parameter SEsc (Scaliness: shows how dry the stratum corneum is being analyzed) was used to evaluate the efficacy of the investigational product to improve the texture (softness/smoothness) of the skin. The lower the SEsc value, the lower the dryness and, therefore, the greater the hydration.

To capture the images, the glass of the device was perpendicularly placed upon the subjects' skin. The measurements follow a previously determined randomization (APPENDIX 4) and was performed at the subject's lower legs at the following time-points: T0=before investigational product use; and T14=after 14 days of investigational product use.

### Self-Assessment Questionnaire Performed by the Study Subjects

The Self-Assessment by the study subjects is performed by following the “Standard Guide for Sensory Claim Substantiation” (ASTM E 1958-06, 2006), by using questionnaires. The ASTM (American Society for Testing and Materials) standards organization has been developed for over a century and represents one of the greatest voluntary organizations for standards development in the world, being a reliable source of technical standards of material, products, systems and services. Known by their high technical quality and relevance on market, ASTM standards have an important role in the infrastructure of the information guiding the study design, product manufacturing and commerce in global economy. The “Standard Guide for Sensory Claim Substantiation” is an ASTM standard that aims to disclose the good practices in sensory studies, approaching reasonable practices for executing sensory studies to validate product claims.

Subjective questionnaires allow the sponsor to gauge the subjects' perceptions of the investigational product and its effects. Statements asked for subjects' agreement to a statement with a six-point scale.

Time-point, statements and scale to be answered by the study subjects are described in Table 24 below.

TABLE 24
Self-Assessment attributes and scale.

| Time-point | Statement | Scale |
|---|---|---|
| T14 | 1. Improves the overall appearance of my skin. | 1 = Agree Strongly |
| | 2. Improves the moisturization of my skin. | 2 = Agree |
| | 3. Skin looks and feels more hydrated. | 3 = Agree somewhat |
| | 4. Skin feels calmer. | 4 = Disagree |
| | 5. Skin looks less red. | somewhat |
| | 6. Skin texture feels and looks smoother. | 5 = Disagree |
| | 7. Skin feels soft. | 6 = Disagree strongly |
| | 8. Product feels gentle on my skin. | |
| | 9. Skin looks brighter (illuminated). | |
| | 10. Skin looks more youthful and healthier. | |
| | 11. Improves the radiance of my skin. | |
| | 12. Improves the evenness of skin. | |
| | 13. Improves the appearance of fine lines and wrinkles* | |
| | 14. I would continue to use this product at the conclusion | |
| | of the study. | |
| | 15. I would recommend this product to friends and family. | |

*Only for face

### Results

### Clinical Grading

At day 14, there was a statistically significant improvement in hydration (visual assessment) (p<0.001) for subjects treated with Formulation 1. Additionally, there was a statistically significant improvement in softness (tactile assessment) (p<0.001), radiance (visual assessment) (p<0.001), texture (tactile assessment) (p<0.001).

### Instrumental, Quantitative Measurements

At 14 days, there was a statistically significant improvement in hydration as measured by Corneometer CM 825 (p=0.026), a statistically significant improvement in scaliness as measured by Visioscan VC 20plus (p=0.001), indicating an improvement in skin texture and hydration.

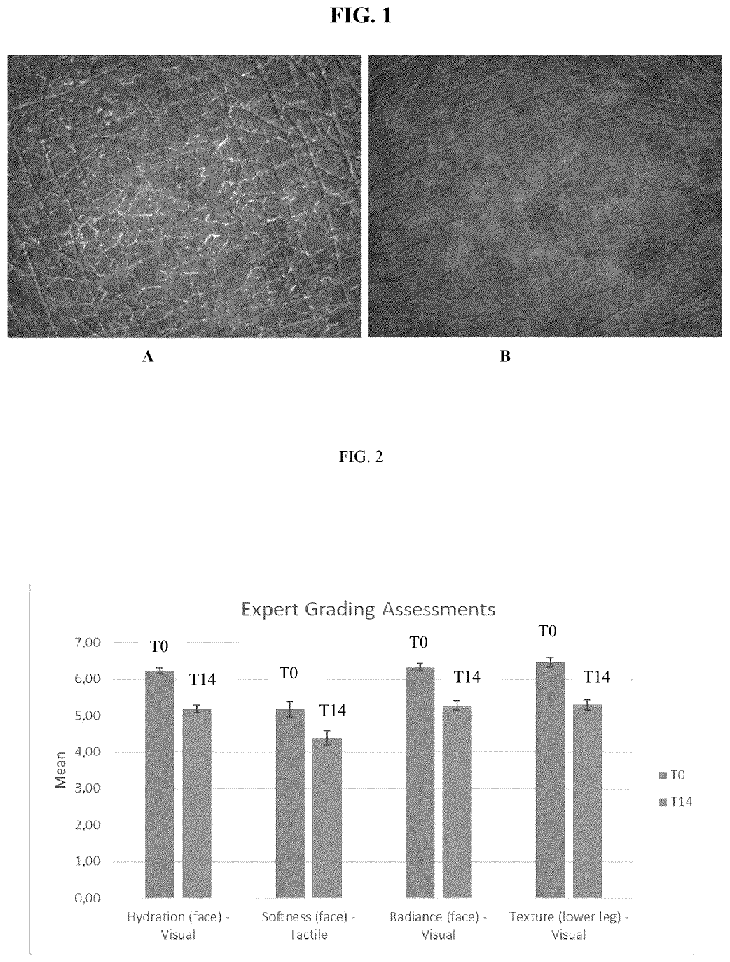

FIG. 1 provides a microscopy image of a subject before treatment with formulation 1 of the present application (panel A), and after treatment with formulation 1 of minimalist hydration formulation of the present application (panel B). After treatment with formulation 1, a marked improvement in skin texture and dryness was observed after 14 days of treatment. Images were taken with LED UV-A light video camera with high resolution to study the skin surface directly.

### Expert Grading Assessments

Expert evaluation indicates a statistically significant improvement of hydration (face), visual, softness (face), tactile and radiance (face), visual and texture (lower leg) as shown in FIG. 2 and summarized herein. Visual improvement was observed after 14 days (T14) of product use in comparison to the initial time-point (T0). 93.5% of subjects showed a statistically significant improvement in hydration of the face (p<0.001*). There was also a statistically significant improvement in softness (tactile) in the face (p<0.001*), radiance in the face (p<0.001*), as well as a statistically significant improvement in texture in the lower leg (p<0.001*) using a Student t test (* significant at 5%).

Referring to FIG. 2, expert evaluation indicates that the product promotes significant improvement of visual (e.g., hydration, radiance, texture) and/or tactile (e.g., softness) aspects of the skin on face and lower legs. After usage of the product for 14 days, subjects are confirmed to have improved hydration, radiance, softness and radiance on face, and improved texture on lower legs. At T14, about 93.5% of subjects had improved hydration on face by about 17.2%, about 67.7% subjects had improved softness and tactile on face by about 14.9%, about 80.6% of subjects had improved radiance on face by about 16.8%, and about 74.2% of subjects had improved texture on lower legs by about 18%, when compared to T0.

### Corneometer CM 825 Measurements

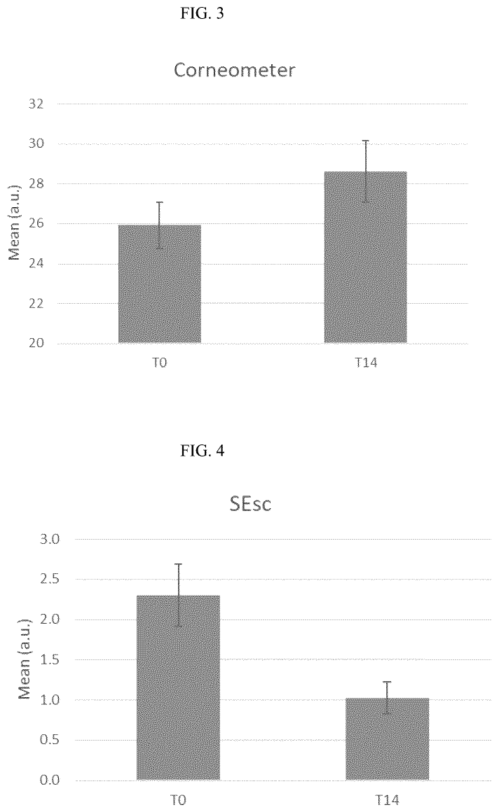

The Conrneometer CM 825 measurements show statistically significant increase of hydration was observed after 14 days (T14) of product use in comparison to the initial time-point (T0) as shown in FIG. 3 (p=0.026). Statistical analysis performed using a Student t test (* significant at 5%).

Referring to FIG. 3, Corneometer measurements indicate that the product promotes significant improvement of skin hydration. After usage of the product for 14 days, a statistically significant increase and improvement of hydration was observed by about 10.4% as quantified by Corneometer measurements when compared to T0 (p=0.026).

### Visioscan VC 20Plus Measurements

The Visioscan VC 20plus measurements show statistically significant reduction of scaliness (SEsc parameter) after 14 days (T14) of product use in comparison to the initial time-point (T0), indicating a reduction of the dryness and scaliness, thus improving the skin texture and hydration (p=0.001*; 87.1% of subjects felt improvement of the skin texture and hydration). The results are shown in FIG. 3.

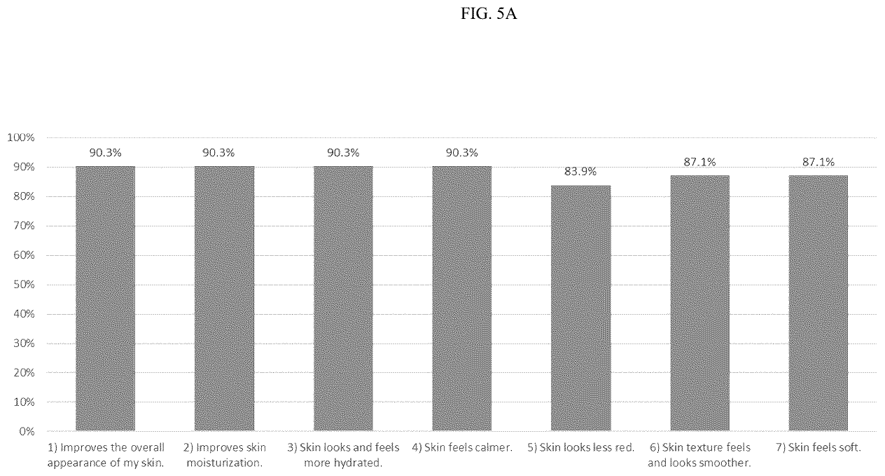

Referring to FIG. 4, Visioscan VC 20plus measurements indicate that the product promotes significant reduction of skin dryness and scaliness, which indicate improvement of the skin texture and hydration. After usage of the product for 14 days, a statistical significant reduction in skin dryness and scaliness was observed (p=0.001; * significant at 5% using a Student t test) About 87.1% of subjects were confirmed to have reduced skin dryness and scaliness (i.e. improved texture and hydration) by about 55.7% as quantified by SEsc parameters when compared to T0.

### Self-Assessment Questionnaire Performed by the Study Subjects

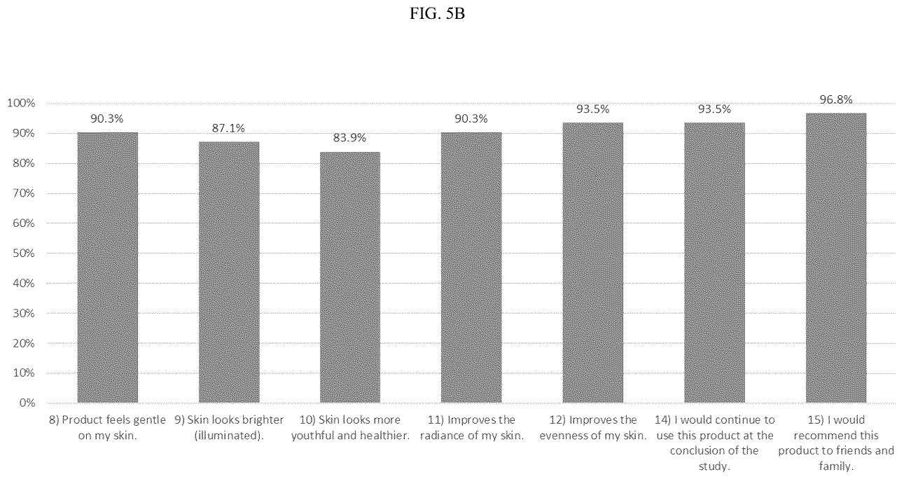

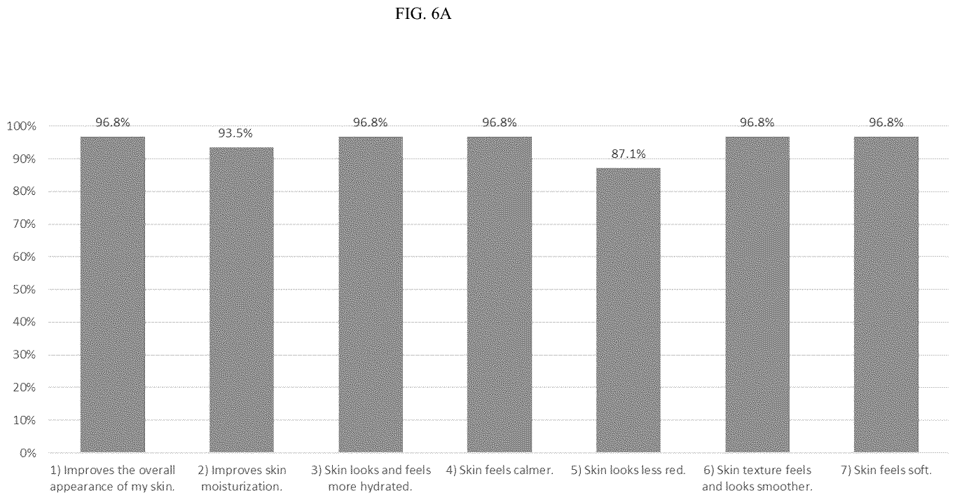

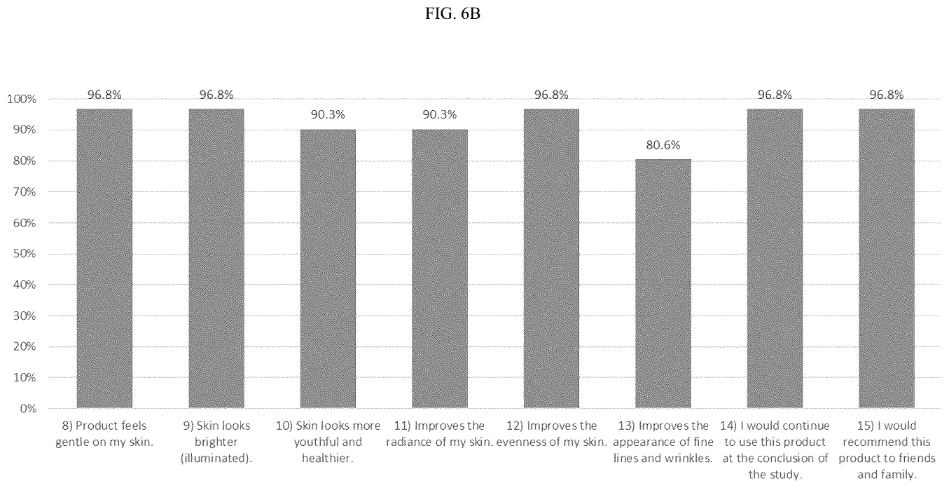

Subjects were asked to complete a self-assessment questionnaire after 14 days of using the product. The results are shown in FIGS. 5A and 5B, and Table 26 with respect to improvement of skin on the face, and in FIGS. 6A and 6B, and Table 25 with respect to improvement of skin in the lower legs.

TABLE 25
Self-assessment questionnaire about lower leg skin

| Statement | Agreement |
|---|---|
| Improves the overall appearance of my skin. | 90.3% |
| Improves skin moisturization. | 90.3% |
| Skin looks and feels more hydrated. | 90.3% |
| Skin feels calmer. | 90.3% |
| Skin looks less red. | 83.9% |
| Skin texture feels and looks smoother. | 87.1% |
| Skin feels soft. | 87.1% |
| Product feels gentle on my skin. | 90.3% |
| Skin looks brighter (illuminated). | 87.1% |
| Skin looks more youthful and healthier. | 83.9% |
| Improves the radiance of my skin. | 90.3% |
| Improves the evenness of my skin. | 93.5% |
| I would continue to use this product at the | 93.5% |
| conclusion of the study. | |
| I would recommend this product to friends | 96.8% |
| and family. | |

TABLE 26
Self-assessment questionnaire about face skin

| Question | Agreement |
|---|---|
| Improves the overall appearance of my skin. | 96.8% |
| Improves skin moisturization. | 93.5% |
| Skin looks and feels more hydrated. | 96.8% |
| Skin feels calmer. | 96.8% |
| Skin looks less red. | 87.1% |
| Skin texture feels and looks smoother. | 96.8% |
| Skin feels soft. | 96.8% |
| Product feels gentle on my skin. | 96.8% |
| Skin looks brighter (illuminated). | 96.8% |
| Skin looks more youthful and healthier. | 90.3% |
| Improves the radiance of my skin. | 90.3% |
| Improves the evenness of my skin. | 96.8% |
| Improves the appearance of fine lines and wrinkles. | 80.6% |
| I would continue to use this product at the | 96.8% |
| conclusion of the study. | |
| I would recommend this product to friends and family. | 96.8% |

Referring to FIGS. 5A and 5B that show improvements on lower leg skin, approximately 90.3% of subjects indicated the product improved their overall appearance of skin, skin moisturization, skin hydration by look and feel, skin calmness by feel, skin radiance, and/or the product feels gentle on their skin. Approximately 83.9% of subjects indicated that their skin looked less red, and/or more youthful and healthier. Approximately 87.1% of subjects indicated that their skin looks brighter (illuminated), and/or the skin texture felt and looked smoother and soft. Approximately 93.5% of subjects indicated the product improves the evenness of their skin. Approximately 93.5% of subjects indicated they would continue to use this product at the conclusion of the study, while approximately 96.8% of subjects indicated they would recommend this product to friends and family.

Referring to FIGS. 6A and 6B that show improvements on face skin, approximately 96.8% of subjects indicated the product improved their overall appearance of skin, skin hydration by look and feel, skin calmness by feel, skin texture feels or looks smoother, skin feels softer, the product feels gentle on their skin, the skin looked brighter (illuminated), and/or skin evenness. Approximately 93.5% of subjects indicated that the product improved their skin moisturization. Approximately 90.3% of subjects indicated that their skin texture felt and looked smoother and soft, and/or had improved skin radiance. Approximately 87.1% of subjects indicated their skin looked less red, while approximately 80.6% of subjects indicated the product improved their appearance of fine lines and wrinkles on their skin. Approximately 96.8% of subjects indicated they would continue to use this product at the conclusion of the study, and/or would recommend this product to friends and family.

In short, majority of subjects agree that usage of the product (Formulation 1) hydrates the skin, leaves the skin soft, leaves the skin smooth, improves skin radiance, and improves skin texture.

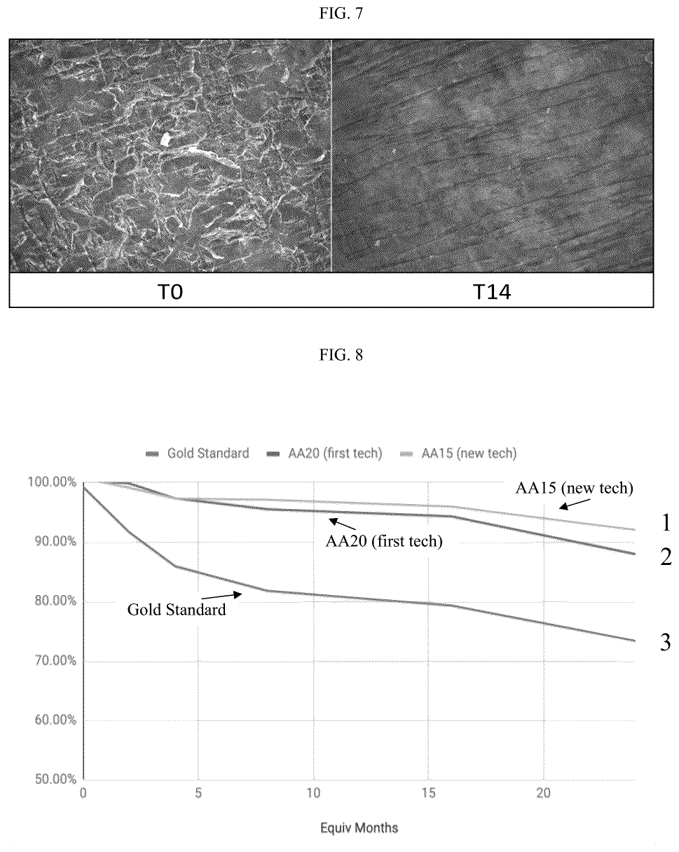

FIG. 7 depicts a representative image of skin improvement on a subject observed on the study. At the initial time of the study (T0), the subject's skin showed obvious dryness and scaliness. After using the product for 14 days (T14), the skin overall appearance had conspicuously improved, looked hydrated, and had less fine lines or wrinkles.

### Conclusions

Improvement in measures of hydration, and texture, as measured by clinician grading and instrumentation, were in agreement. Clinician grading also revealed significant improvements in skin softness and radiance. Furthermore, assessments revealed subjective improvements in overall appearance of skin, moisturization, hydration, calmness, redness, texture, softness, brightness, youthful and healthy appearance, radiant, evenness, and appearance of fine lines and wrinkles.

Self-Assessment by Patients after 14 Days of Use:

97% of participants, after a 14 day treatment with Formulation 1 agreed that:
the overall appearance of skin was improved;
skin looked and felt more hydrated;
skin felt calmer;
skin texture looked and felt smoother;
skin looked brighter;
skin tone evenness was improved; and
skin looked more youthful and healthier.

90% of participants, after a 14-day treatment with Formulation 1, agreed that skin radiance was improved.

### Example 6: Vitamin C Formulations—Assessment of Formulation Components

A series of experiments were performed to assess and optimize the components of the subject vitamin C formulations. AA refers to L-ascorbic acid. U refers to urea. % values are wt %

### Summary of Experiments:

### Ascorbic Acid to Urea:

The maximum amount of AA solubilized in 1,3-propanediol before recrystallization was ˜12%. This solubility limit was also observed for propylene glycol (1,2 propanediol).

First: completely solubilized AA/U in 1,3 propanediol at 20% AA, and 15% U.
Reduced to 10% U content, and still no recrystallization.
Reduced to 5% U content, and recrystallization occurred.
Tested 8% U content and recrystallization occurred.
10% U content thus appeared to be close to the minimum amount U required to solubilize 20% AA.
U in combination with a 15% AA content:
5% U prevented recrystallization
3.75% U prevented recrystallization
2.5% U resulted in recrystallization
Maximum saturation level experiments
30% AA, 20% U in 1,3 propanediol resulted in recrystallization
28% AA, 20% U resulted in fully solubilized AA with no recrystallization
The limitation of this composition is the solubility of U in 1,3 propanediol-˜27.8% saturation can be reached before recrystallization of U becomes apparent

Using these numbers is how the following equation was obtained for determining the amount of U, and thus the ratio of AA to U in high concentration ascorbic acid formulations:

(AA-X)*1.25=U %, where X=the maximum % solubility of AA in the chosen solvent. In this case, X=12%, as noted above.

The equation is relevant to compositions including a lower limit of 5% ascorbic acid because the inclusion of other polyols that provide very low or virtually no solubility of AA, such as dimethyl isosorbide (DMI). Therefore, when a mixture of propanediol and DMI is used as the solvent, for example, the X value can be 5% (maximum solubility of AA), depending on the ratio of propanediol and DMI used.

### Solvents

1,3 propanediol, 1,2 propanediol, butylene glycol, pentylene glycol, and hexanediol were identified as preferred solvents. 1,3 propanediol (trade name: Zemea) is inherently different from and preferable to the various polyols described. Below is a review of various polyols and reasons why 1,3 propanediol is unique and preferable:

1,3-propanediol, sometimes referred to in the art as propanediol, is unique in that it possesses a combination of gentleness on skin (even applied neat, or at 100% concentration), relatively low viscosity (and therefore perceived “lightness” on skin), environmental friendliness (not petroleum-derived), natural derivation (corn or sugar cane), low odor, and moderate ability to solubilize ascorbic acid.

1,2-propanediol, otherwise referred to in the art as propylene glycol, although of low viscosity and possessing a moderate ability to solubilize ascorbic acid, is well-known for inducing skin irritation and sensitivity. Additionally, it is derived from petroleum and possesses an unpleasant odor, reminiscent of acetone.

1,3-butanediol, otherwise referred to in the art as butylene glycol, is of low viscosity, possesses a moderate ability to solubilize ascorbic acid, and is relatively gentle on skin. However, like propylene glycol, it is derived from petroleum (not environmentally friendly) and possesses an unpleasant odor, reminiscent of acetone. also applicable to dipropylene glycol

1,5-pentanediol, otherwise referred to in the art as pentylene glycol, possesses a moderate ability to solubilize ascorbic acid, low odor, and certain versions are not derived from petroleum but from sugarcane or corn. However, upon application to skin, it imparts a “heavier”, less desirable texture on skin. Additionally, its recommended use level is capped at 5%, limiting usage as a primary solvent.

1,2-hexanediol possesses a moderate ability to solubilize ascorbic acid. However, upon application to skin, it imparts a “heavier”, less desirable texture on skin, possesses an unpleasant odor reminiscent of acetone, and is derived from petroleum. Additionally, its recommended use level is capped at 10%, limiting usage as a primary solvent.

Glycerin and diglycerin, possess a moderate ability to solubilize ascorbic acid, are relatively gentle on skin, are low-odor, and are not derived from petroleum. However, they are of a very viscous nature, and impart not only an undesirable, “heavy” texture on skin, but one that is exceedingly sticky.

Dimethyl isosorbide is relatively gentle on skin and not derived from petroleum, and imparts a “light”, not undesirable texture when applied to skin. However, it has a very limited ability to solubilize ascorbic acid and possesses a slight, but noticeable chemical odor reminiscent of chlorine.

### Urea Agents

Urea is preferable to hydroxyethyl urea. There are a number of reasons for this:
Urea, when used in sufficient low concentrations (10-15% and below) in leave-on applications, possesses desirable humectant, barrier-repairing and very mild keratolytic properties, which in combination are very effective at improving the feel and look of dry and/or rough skin.
Urea is naturally present not only in the human body but specifically in the skin, where it acts as a natural moisturizing factor (NMF).
Hydroxyethyl urea possesses similar humectant properties, but not the same level of barrier-repairing and mild keratolytic properties of urea.
Additionally, hydroxyethyl urea may contain trace amounts of diethanolamine, which is listed as a potential carcinogen by California's Proposition 65, and requires a warning on products sold to consumers. For this reason, at least one manufacturer of hydroxyethyl urea has stated that it will discontinue production of this ingredient (AkzoNobel).

### Optional Additional Components

Additional ingredients were chosen for their compatibility with (e.g., miscibility in) 1,3 propanediol, 1,2 propanediol, and 1,3 butanediol. Additional notes and observations on each optional additional component are shown below.

### Panthenol (Pro-Vitamin B5)

This is a humectant that shows soothing and moisturizing properties for skin. Both enantiomers, D-panthenol and L-panthenol, are potent humectants. However, only D-panthenol is converted into pantothenic acid in the skin, which confers additional benefits to skin (wound healing, for example).
Research shows that it can reduce irritation to skin by other ingredients
Research also shows barrier-repairing ability (stimulation of physiologic lipid synthesis)
DL-panthenol is a racemic mixture of the two enantiomers; it is in powdered/crystal form.
D-panthenol is a viscous liquid.
DL-panthenol is freely soluble in 1,3 propanediol, 1,2 propanediol and 1,3 propanediol (up to 50%)
D-panthenol is also freely soluble in 1,3 propanediol, 1,2 propanediol and 1,3 propanediol, with no risk of recrystallization at any concentration (as it is already liquid at room temperature).

Inhibition of transepidermal water loss is apparent at concentrations of 1% and above.

### Hyaluronic Acid

Hyaluronic acid is a humectant that shows the ability to form a viscoelastic film on skin that prevents transepidermal water loss.

It is usually incorporated in aqueous solutions in its salt form, sodium hyaluronate

However, there is a raw material blend that is largely free from water, in which it is incorporated in a vehicle of glyceryl polymethacrylate, butylene glycol (1,3 butanediol), and natto gum (trade name Hydrafilm 3 MW by The Innovation Company). This makes it compatible with the nonaqueous formulations of the present disclosure.

Documents from The Innovation Company show usage of this material up to 9.1% by weight of the final formula.

The chemical composition is as follows:
75-85% glyceryl polymethacrylate
15-20% butylene glycol
0.5-2% natto gum
0.5-2% hyaluronic acid.

### Pinus Pinaster Bark Extract

Components of the bark extract of pinus pinaster species show the ability to recycle vitamin C.

Additionally, there is research to show their general antioxidant, anti-inflammatory and anti-acne properties.

pycnogenol may be used as an alternative when pinus pinaster bark extract is desired.

A material blend from Kinetik called Pantrofina Skin360 (PS360) is utilized in the subject formulations.

PS360, unlike pycnogenol, is already in liquid form as it uses diglycerin as a solvent, making it very easy to incorporate.

Additionally, Res Pharma Industriale provides in-vitro and clinical data to show effectiveness against free radical damage, inflammation and acne at a concentration of 0.5% by weight of PS360.

The chemical composition is as follows:
90-95% diglycerin
5-10% pinus pinaster bark extract

### Madecassoside

Centella Asiatica extract is often used for its soothing properties.

Madecassoside is a highly purified glycosylated triterpene of Centella Asiatica. It is sold by raw material supplier SEPPIC, who share in-vitro and clinical data showing its anti-inflammatory and other effects on skin.

This is a very expensive ingredient ($6.10 per gram), but clinical data from SEPPIC shows desirable ability to reduce erythema (skin redness) in concentrations of 0.2%.

At a concentration of 0.2%, madecassoside is soluble in 1,3 propanediol, 1,2 propanediol and 1,3 butanediol.

In some embodiments, the madecassoside is madecassoside asiaticoside.

### Azelaic Acid

Azelaic acid (AzA) is well studied for its ability to treat acne, rosacea and melasma, due to the fact that it was studied and sold as a prescription drug. Though poorly understood, these effects are believed to be a result of AzA's anti-bacterial, anti-inflammatory, and keratolytic effects, as well as its unique ability to cause apoptosis in abnormal melanocytes.

It is very poorly soluble in most solvents. As a result, all products currently on the market, both prescription and cosmetic, are sold as opaque emulsions, where the AzA is not solubilized but instead finely milled into a powder and suspended in the viscous vehicle.

Because of an inability to solubilize AzA, a preferred component for maximizing delivery into the skin of active ingredients, the team behind prescription product Finacea (currently considered to be the gold standard) chose to manipulate pH, as they discovered that, counterintuitively, a salt form of AzA (formed in aqueous environments in which the pH is higher than the pKa of AzA, 4.15), is slightly better at penetrating skin.

The present inventor discovered that AzA can be solubilized in 1,3 propanediol at relatively high concentrations—up to 10%.

The solubility of AzA in 1,3 propanediol can be slightly increased by the presence of hydroxyethyl urea.

For example, it is possible to solubilize 7.5% AzA with 10% AA, 5% U in a 1,3 propanediol base.

### Cinnamic Acids and Derivatives Thereof

Cinnamic acid and derivatives thereof work synergistically with ascorbic acid to provide additional antioxidant protection to skin. Cinnamic acids of interest and sources thereof include, but are not limited to, ferulic acid, caffeic acid and coumaric acid. In some embodiments, the cinnamic acid is ferulic acid. The cinnamic acid ingredient used in the subject formulation can be a combination of ferulic acid and/or substituted cinnamic acids. For example, the cinnamic acid agent used can be a combination of ferulic acid and caffeic acid.

### Ferulic Acid

Ferulic acid is an antioxidant that increases AA's photoprotective effect on skin. It can also somewhat stabilize AA in aqueous systems.

Ferulic acid is readily soluble in 1,3 propanediol, 1,2 propanediol, 1,3 butanediol and dimethyl isosorbide can increase the effectiveness of ferulic acid by enhancing skin penetration.

Stabilizes AA and provides antioxidant activity and photoprotection.

### Acetyl Zingerone

Acetyl zingerone is a broad-spectrum antioxidant that can prevent lipid peroxidation. It was engineered to be a more stable, more potent derivative of zingerone.

Sytheon provides in-vitro and clinical data showing its antioxidant, photoprotective, and anti-aging properties.

Acetyl zingerone may be used as a replacement for tocopherol.

Acetyl zingerone is readily soluble in 1,3 propanediol, 1,2 propanediol and 1,3 butanediol at the desired concentrations (0.5-1%), eliminating the need for emulsifiers as would be required for tocopherol.

### Glycyrrhizic Acid

Glycyrrhizic acid, like many other derivatives from licorice root (Glycyrrhiza Glabra, Glycyrrhiza Uralensis), shows anti-inflammatory, antioxidant and skin lightening properties.

Unlike 18B-glycyrrhetinic acid, glycyrrhizic acid shows solubility in 1,3-propanediol. Other derivatives of licorice root can be use, such as dipotassium glycyrrhizate, monoammonium glycyrrhizate, etc.

### Example 7: Exemplary Vitamin C Formulations

The exemplary formulations of Table 27 were prepared and assessed.

TABLE 27
Exemplary Vitamin C Formulations 1A-3A

| Formulation | 1,3-propanediol | urea | L-ascorbic acid | ferulic acid |
|---|---|---|---|---|
| 1A | 69.5% | 10% | 20% | 0.5% |
| (Serum) | | | | |
| 2A | 51.5% | 20% | 28% | 0.5% |
| (Rinse-Off Mask) | | | | |
| 3A | 79% | 5% | 15% | 1% |
| (Serum) | | | | |

### Example 8: Exemplary Vitamin C Formulations

### Ratio of Ascorbic Acid to Urea

In order to determine a desirable ratio of ascorbic acid to urea for the compositions of this disclosure, the maximum concentration for ascorbic acid that can be solubilized is first determined, with heat exposure (not exceeding 80° C. in order to prevent degradation of ascorbic acid), in a given solvent without precipitation upon cooling. Experiments revealed this concentration to be approximately 10-12% for 1,3 propanediol, propylene glycol (1,2 propanediol) and butylene glycol (1,3 butanediol), and significantly lower for dimethyl isosorbide.

Next, concentrations of ascorbic acid beyond the aforementioned maximum concentration are solubilized, using urea as a co-solvent. Repeated experiments of this nature, using differing concentrations and ratios of urea to ascorbic acid, revealed the following relationship between these two substances (ascorbic acid and urea) that is useful to create fully solubilized composition which is storage stable:

$$( AA - X ) * 1.25 = U$$

AA=concentration of ascorbic acid
X=maximum solubilization point of ascorbic acid in solvent of choice
U=concentration of urea

Compositions having an ascorbic acid concentration as low as 5% can be prepared in cases where the polyol solvents used provide very low solubility, such as dimethyl isosorbide (DMI). Therefore, a mixture of propanediol and DMI, for example, can yield an X value of 5% (maximum solubility of AA), depending on the ratio of propanediol and DMI.

In general, 1,3 propanediol is preferred over 1,2 propanediol, butylene glycol, pentylene glycol, or hexanediol. 1,3 propanediol is preferable to various polyols described in the art. Below is a review of various polyols and reasons why 1,3 propanediol is unique and preferable:

1,3 propanediol, otherwise referred to in the art as propanediol, is unique in that it possesses a combination of gentleness on skin (even applied neat, or at 100% concentration), relatively low viscosity (and therefore perceived “lightness” on skin), environmental friendliness (not petroleum-derived), natural derivation (corn or sugar cane), low odor, and moderate ability to solubilize ascorbic acid.

1,2 propanediol, otherwise referred to in the art as propylene glycol, although of low viscosity and possessing a moderate ability to solubilize ascorbic acid, induces skin irritation and sensitivity. Additionally, it is derived from petroleum and possesses an unpleasant odor, reminiscent of acetone.

1,3 butanediol, otherwise referred to in the art as butylene glycol, is of low viscosity, possesses a moderate ability to solubilize ascorbic acid, and is relatively gentle on skin. However, like propylene glycol, it is derived from petroleum (not environmentally friendly) and possesses an unpleasant odor, reminiscent of acetone.

Note that these properties also apply to dipropylene glycol.

1,5 pentanediol, otherwise referred to in the art as pentylene glycol, possesses a moderate ability to solubilize ascorbic acid, low odor, and certain versions are not derived from petroleum but from sugarcane or corn. However, upon application to skin, it imparts a “heavier”, less desirable texture on skin. Additionally, its recommended use level is generally capped at 5%, limiting usage as a primary solvent.

1,2 hexanediol possesses a moderate ability to solubilize ascorbic acid. However, upon application to skin, it imparts a “heavier”, less desirable texture on skin, possesses an unpleasant odor reminiscent of acetone, and is derived from petroleum. Additionally, its recommended use level is capped at 10%, limiting usage as a primary solvent.

Glycerin and diglycerin, possess a moderate ability to solubilize ascorbic acid, are relatively gentle on skin, are low-odor, and are not derived from petroleum. However, they are highly viscous, and impart not only an undesirable “heavy” texture on skin, but one that is exceedingly sticky.

Dimethyl isosorbide is relatively gentle on skin and not derived from petroleum, and imparts a “light”, not undesirable texture when applied to skin. However, it has a very limited ability to solubilize ascorbic acid and possesses a slight, but noticeable chemical odor reminiscent of chlorine.

Urea is preferable to hydroxyethyl urea. There are a number of reasons for this, as summarized below:

Urea, when used in sufficient low concentrations (10-15% and below) in leave-on applications, possesses desirable humectant, barrier-repairing and very mild keratolytic properties, which in combination are very effective at improving the feel and look of dry and/or rough skin. Urea is naturally present not only in the human body but specifically in the skin, where it acts as a natural moisturizing factor (NMF).

Hydroxyethyl urea possesses similar humectant properties, but not the barrier-repairing and mild keratolytic properties of urea. Additionally, hydroxyethyl urea may contain trace amounts of diethanolamine, a potential carcinogen.

Additional ingredients can be included which are compatible with the ascorbic acid/solvent/urea combination of interest.

The exemplary formulations of Table 28 were prepared and assessed as having desirable properties including storage stability.

TABLE 28
Components of Exemplary Compositions (% by weight)

| Formulation | 1A | 2A | 3A | 4A | 5A | 6A | 7A | 8A |
|---|---|---|---|---|---|---|---|---|
| ascorbic acid | 5% | 5% | 10% | 15% | 15% | 20% | 25% | 20% |
| urea/ | 5% | 5% | 3% | 8% | 10% | 10% | 20% | 10% |
| hydroxyethyl | | | | | | | | |
| urea | | | | | | | | |
| Ferulic acid | 0.5% | 0.5% | 0.5% | 1% | 0.5% | 0.5% | | 0.5% |
| C3-C6 polyol | 83.8% 1,3- | 53.3% 1,3 | 75.3% 1,3- | 72.9% 1,3- | 48% 1,3 | 69% 1,3- | 53% 1,3 | 60% 1,3 |
| | propanediol | propanediol | propanediol | propanediol | propanediol | propanediol | propanediol | propanediol |
| Additive 1 | 5% panthenol | 5% panthenol | 7.5% azelaic | .5% acetyl | 1% tocopherol | 0.5% pinus | 2% salicylic | |
| | | | acid | zingerone | | pinaster | acid | |
| | | | | (trade name | | extract | | |
| | | | | Synoxyl AZ) | | (diglycerin, | | |
| | | | | | | pinus pinaster | | |
| | | | | | | bark extract; | | |
| | | | | | | trade name | | |
| | | | | | | Pantrofina | | |
| | | | | | | Skin360) | | |
| Additive 2 | 0.2% | 0.2% | 0.5% pinus | 0.5% pinus | .5% Bis- | | | 0.5% |
| | madecassoside | madecassoside | pinaster | pinaster | Ethylhexyl | | | diglycerin, |
| | | | extract | extract | Hydroxydi- | | | Pinus |
| | | | (diglycerin, | (diglycerin, | methoxy | | | Pinaster |
| | | | pinus pinaster | pinus pinaster | Benzylmalonate | | | Bark |
| | | | bark extract; | bark extract; | (trade name | | | Extract |
| | | | trade name | trade name | Ronacare AP) | | | |
| | | | Pantrofina | Pantrofina | | | | |
| | | | Skin360) | Skin360) | | | | |
| Additive 3 | 0.5% pinus | 0.5% pinus | 0.2% | 5% panthenol | 5% silicone- | | | |
| | pinaster | pinaster | madecassoside | | based emulsifier | | | |
| | extract | extract | | | (dimethicone, | | | |
| | (diglycerin, | (diglycerin, | | | dimethicone/ | | | |
| | pinus pinaster | pinus pinaster | | | peg-10/15 | | | |
| | bark extract; | bark extract; | | | crosspolymer; | | | |
| | trade name | trade name | | | trade name | | | |
| | Pantrofina | Pantrofina | | | KSG-280) | | | |
| | Skin360) | Skin360) | | | | | | |
| Additive 4 | | 15% dimethyl | 3% panthenol | 0.1% | 5% silicone- | | | |
| | | isosorbide | | madecassoside | based emulsifier | | | |
| | | | | | (Lauryl PEG-9 | | | |
| | | | | | Polydimeth- | | | |
| | | | | | ylsiloxyethyl | | | |
| | | | | | Dimethicone) | | | |
| Additive 5 | | 30% silicone- | | | 15% isododecane | | | |
| | | based emulsifier | | | | | | |
| | | (dimethicone, | | | | | | |
| | | dimethicone/ | | | | | | |
| | | peg-10/15 | | | | | | |
| | | crosspolymer; | | | | | | |
| | | trade name | | | | | | |
| | | KSG-280) | | | | | | |
| Additive 6 | | .5% Bis- | | | | | | |
| | | Ethylhexyl | | | | | | |
| | | Hydroxydi- | | | | | | |
| | | methoxy | | | | | | |
| | | Benzylmalonate | | | | | | |
| | | (trade name | | | | | | |
| | | Ronacare AP) | | | | | | |

Other variations: dimethyl isosorbide, caprylyl glycol or decylene glycol can be utilized as an alternative or additional solvents in the compositions of Table 27 or Table 28.

### Example 9: Storage Stability Studies of Vitamin C Formulation

### Stability Method

Samples are stored in sealed containers, sealed from the atmosphere, at 40 degrees Celsius for up to 12 weeks. Results at 0 to 8 weeks are shown in Table 4. In general, 8 weeks storage under these conditions is expected to be equivalent to storage for 16 months at room temperature. The compositions in the containers are sampled at each time point, and assessed for levels of degradation of vitamin C using HPLC analysis.

### Compositions

Exemplary compositions were prepared containing either approx. 20% vitamin C (Formulation 6A referred to in Table 28)

The storage stability of these compositions was compared to control compositions that included clinically comparable amount of vitamin C (15%) dissolved in water with the addition of a ferulic acid in a concentration of 0.5%, tocopherol in a concentration of 1%, with additional components of a glycol ether, alkanediol, laureth-23, panthenol, triethanolamine, phenoxyethanol, and sodium hyaluronate. The results are shown in Table 29. The exemplary serum (approx. 15% vitamin C) compositions are still within specification after weeks 8 of testing (or the equivalent to 16 months at room temperature), as opposed to the control compositions which fell out of specification (OOS) by week 2 of testing (or equivalent to 4 months at room temperature).

TABLE 29
Storage stability

| Storage time | | % vitamin C by HPLC | |
|---|---|---|---|
| Week (40° C.) | Equiv. Months (RT) | Serum | Serum Control |
| 0 | 0 | 100.35% | 99.20% |
| 1 | 2 | 99.80% | 91.60% |
| 2 | 4 | 97.30% | *85.87% |
| 4 | 8 | 95.45% | *81.73% |
| 8 | 16 | 94.25% | *79.27% |

* indicates the samples were assessed as being OOS according to Out of Specification (OOS) Standards: 90% or less vitamin C stability.

### Example 10: Comparative Studies of Vitamin C Formulation

U.S. Pat. No. 6,020,367 (patent '367), which is incorporated herein in its entirety, attempted to show the viability of “supersaturated solutions” of vitamin C in a polyol. Several compositions of patent '367 were prepared in accordance with the disclosure. However, many of the “supersaturated solutions” of vitamin C patent '367 do not actually remain solubilized at room temperature over time. Rather, the solutions lead to development of vitamin C crystals which at first create a cloudy appearance and then settle downward. Such compositions are non-uniform and unsuitable for use as end products.

### Glycerin Solvent

A mixture of 25% ascorbic acid and 75% glycerin was prepared. The ascorbic acid was and solubilized with heating at 95° C. to produce a transparent solution. Upon cooling to room temperature, crystallization became apparent within the first 24 hours of storage.

### Butylene Glycol Solvent

According to patent '367 butylene glycol has a lower ability to solubilize ascorbic acid.

A mixture of 25% ascorbic acid and 75% butylene glycol was prepared. Even with heating at the maximum temperature of 95° C. (under agitation), butylene glycol failed to solubilize the ascorbic acid content, leaving a “cloudy” appearance and sedimentation upon cessation of agitation.

### Propylene Glycol Solvent

According to patent '367 propylene glycol has the lowest ability of these solvents to solubilize ascorbic acid. A mixture of 25% ascorbic acid and 75% propylene glycol was prepared. The ascorbic acid was and solubilized with heating at 95° C. to produce a transparent solution. Upon cooling to room temperature, crystallization became apparent within the first 24 hours of storage.

It is important to note the fragile nature of ascorbic acid renders it sensitive not only to the presence of water and air, but also heat. When heated above 80° C., even in anhydrous vehicles such as polyols, there is a risk for degradation of the ascorbic acid. The solutions described above prepared according to the direction of patent '367, when heated to the described range of 85-95° C., showed signs of degradation.

U.S. Publication No. 2007/0077261 (publication '261), which is incorporated herein in its entirety, discloses compositions including broad ranges of ascorbic acid and urea, but fails to identify both the “floor” (minimum amount of urea required to solubilize a certain amount of ascorbic acid) and the “ceiling” (maximum amount of ascorbic acid that can be solubilized through this method).

Example 3 of publication '261 discloses a composition including: 50% propylene glycol, 22% urea and 28% ascorbic acid, heated to 75° C. with agitation until transparent, then cooled to room temperature. This example was reproduced. The solution started to precipitate within 24 hours, demonstrating a failure to understand and elucidate the required ratio of urea to ascorbic acid.

Using the equation of this disclosure set forth above, the correct concentration of urea to solubilize 28% ascorbic acid in propylene glycol would be 20% (the proper “floor”). Indeed, a solution of 28% ascorbic acid and 20% urea in propylene glycol was prepared and remained fully solubilized even after 30 days of storage at room temperature. Furthermore, experiments reveal that these concentrations of ascorbic acid (28%) and urea (20%), also represent the maximum concentrations soluble in propylene glycol, butylene glycol and propanediol, before urea itself starts to precipitate in solution (the “ceiling”).

Experiments showed that no concentration of urea within the 5-40% range can solubilize 40% ascorbic acid in a polyol base:
40% ascorbic acid, 5% urea, 55% propylene glycol
40% ascorbic acid, 10% urea, 50% propylene glycol
40% ascorbic acid, 20% urea, 40% propylene glycol
40% ascorbic acid, 40% urea, 20% propylene glycol

All mixtures were heated to 85° C. However, none were solubilized even after agitation at maximum temperature of 85° C. In addition, the urea content disclosed in several examples of publication '261 is not only unnecessarily high (likely because of a failure to identify the “floor”), but also renders the compositions unusable as leave-on facial products and result in irritation of the skin such as burning and irritation, etc. These compositions, when applied to the face, produce an intense burning and stinging sensation that is immediately apparent. This is likely due to urea's keratolytic properties. Additionally, the urea content disclosed in several examples of publication '261 precipitated out of the formulation. In leave-on products intended for the face, maximum urea content is usually 10-15%. Higher concentrations of urea in leave on products than necessary can result of burning sensation of the skin.

Alternatively, formulation 5A of Table 28 and formulation 2A of Table 27 of the present disclosure is identified as a rinse-off product.

### Example 11: AA20 (Vitamin C Formulation) and AA15 (Vitamin C Formulation) Retains Vitamin C Stability Better than Benchmark Technologies

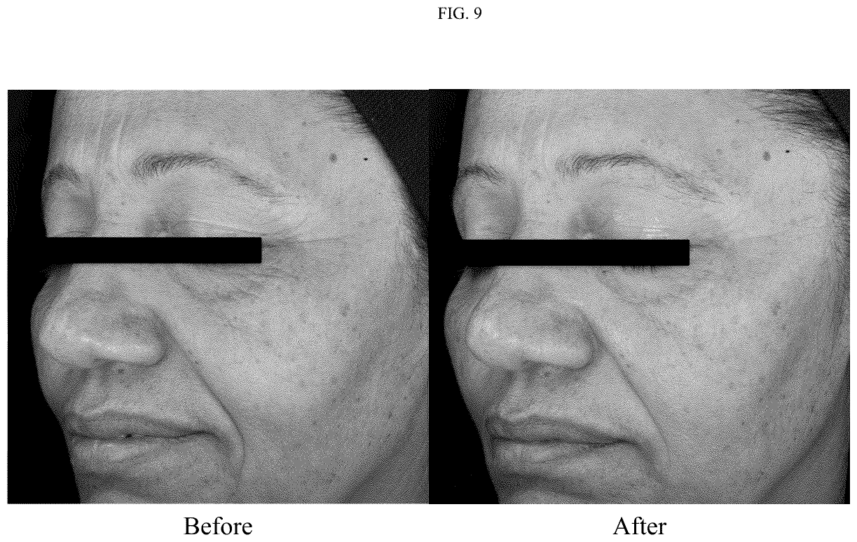

In a head-to-head stability test, rete containing 20% vitamin C (formulation 6A) of vitamin C concentration in the AA20 regimen (second technology: line 1) was compared with the AA20 (first technology: line 2) and the current benchmark technology (gold standard: line 3). The current benchmark technology showed significant degradation over a test period of 24 months, losing nearly 20% vitamin C concentration within the first 8 months of storage (in an unopened container) and nearly 30% after 24 months as shown in FIG. 8 and summarized in Table 30. The first technology (line 2) fared significantly better, losing less than 6% vitamin C concentration even after 16 months, and only ˜12% after 24 months. The second technology (line 1) performed the best, losing only 8% vitamin C concentration even after 24 months. In short, both the first and second technologies have superior vitamin C stability retention when compared to the current benchmark technology.

TABLE 30
Comparison of vitamin C concentration stability

| Equiv | | | |
|---|---|---|---|
| Months | Gold Standard | First Tech | Second Tech |
| 0 | 99.20% | 100.35% | 100.67% |
| 2 | 91.60% | 99.80% | 99.07% |
| 4 | 85.87% | 97.30% | 97.27% |
| 8 | 81.73%* | 95.45% | 97.07% |
| 16 | 79.27%* | 94.25% | 95.93% |
| 24 | 73.33%* | 87.90% | 92.00% |

### Example 12: Clinical Use of the Vitamin C Formulation

Pilot Clinical Study with Vitamin C Formulation (Ascorbic Acid 20 (AA20—Formulation 6A of Table 28)

The aim of this study was to determine if the topical use of the Vitamin C formulation could increase skin brightness and result in clinically visible differences in facial hyperpigmentation.

The AA20 (also used interchangeably herein as Vitamin C20, and formulation 6A of Table 28) formulation of Study 1 and Study 3 includes the following components:
69% by weight Propanediol;
10% by weight Urea;
20% by weight Ascorbic Acid;
0.5% by weight ferulic acid; and
0.5% by weight Diglycerin, Pinus Pinaster Bark Extract.

The study details are provided below:
35 patients, all female with light to medium skin tones, with an average age of 48.63, were included in this trial.
Patients were instructed to apply product twice daily in a double-blinded manner.
17 patients applied the vitamin C formula containing 20% ascorbic acid, ferulic acid, and pinus pinaster bark extract in an anhydrous vehicle.
18 patients applied the inactive vehicle.
All patients were given generic moisturizer, cleanser and sunscreen as adjunct products.
Clinical evaluation of pigmentation was performed prior to the study and at weeks 2, 6 and 12.
Direct visual assessment of pigmentation, assessment of skin brightening by Chromameter, self-assessment by patients, and acquisition of facial images by VISIA-CR were performed.

### Results

After 6 weeks of use:
94% of participants agreed that the overall appearance of skin was improved.
94% of participants agreed that skin tone was brighter.
88% of participants agreed that radiance and clarity of skin was increased.
88% of participants agreed that skin texture was smoother.

After 12 weeks of use:
88% of participants agreed that lines and wrinkles were less visible.
88% of participants agreed that skin looked more youthful and healthy

A statistically significant improvement of the vitamin C-treated group was seen in the hyperpigmentation scores at weeks 6 (p=0.039) and week 12 (p=0.007) as measured by clinical visual assessment.

A statistically significant improvement of the vitamin C-treated group was also seen in the pigmentation scores at weeks 6 (p=0.016) and week 12 (p=0.005) as measured by instrumentation (Chromameter).

Skin brightening as measured by clinician grading and instrumentation were in agreement.

There was no significant improvement in the negative control group at any time point as measured by either clinician grading or instrumentation (Chromameter).

Furthermore, self-assessment revealed subjective improvements in overall appearance of skin, uneven skin tone/discoloration, radiance and clarity of skin, appearance of brown spots, lines and wrinkles, skin texture, skin brightness, appearance of skin youth/health, and skin firmness.

### Unexpected Finding:

No adverse skin reactions occurred in any of the patients in the active group.

Two patients from the placebo group experienced adverse skin reactions and were withdrawn from the study. One patient from the placebo group reported symptoms of temporal burning, dryness after absorption and prickling. One patient from the placebo group reported symptoms of temporal burning, erythema, dryness after absorption, prickling and swelling.

### Example 13: Clinical Trial of Regimen Study to Assess Physical Properties of Skin Using a Combination of a Vitamin C Formulation and Minimalist Hydrating Formulation

This follow-up clinical trial was conducted to investigate whether a regimen consisting of the novel vitamin C preparation (Formulation 6A of Table 28) in combination with the Minimalist Hydrating Cream (MHC) (Formulation 1 of Table 1) can improve major sings of photo-aging, including wrinkling, hyperpigmentation, firmness and elasticity.

The AA20 (also used interchangeably herein as Vitamin C20, and formulation 6A of Table 28) formulation of Study 1 and Study 3 includes the following components:
69% by weight Propanediol;
10% by weight Urea;
20% by weight Ascorbic Acid;
0.5% by weight ferulic acid; and
0.5% by weight Diglycerin, Pinus Pinaster Bark Extract.

The minimalist hydration formulation (MHC) used in Study 2 and Study 3 is Formulation 1 of Table 1.

In Summary, human subjects were instructed to first apply the MHC formulation, followed by the AA20 formulation (vitamin C formulation) once daily. Clinical signs of skin aging was assessed prior to the study and at days 14, 30 60 and 90 after commencement of the study. Clinician grading was used to measure wrinkles/fine lines (visual), firmness (visual), elasticity (tactile), skin tone homogeneity/evenness (visual) and dark spots intensity (visual). Instrumental measurements were used to measure skin brightness (Chroma Meter), skin firmness (Cutometer), wrinkle visibility (Visia CR), wrinkle occupancy rate (Visia CR), and skin tone homogeneity (Visia CR). Subjective self-assessments of skin appearance were collected.

A total of 29 subjects, ranging from light to dark skin types, aged between 47 to 59 years old (mean age of 53), demonstrating mild to moderate signs of photo-aging completed the study. Subjects were instructed to first apply Minimalist Hydrating Cream, following by the Ascorbic Acid 20 product once daily. All subjects were given generic cleanser and sunscreen as adjunct products.

Clinical evaluation of signs of skin aging was performed prior to the study and at days 14, 30, 60 and 90. Clinician grading was used to measure wrinkles/fine lines (visual), firmness (visual), elasticity (tactile), skin tone homogeneity/evenness (visual) and dark spots intensity (visual). Instrumental measurements were used to measure skin brightness (Chroma Meter), skin firmness (Cutometer), wrinkle visibility (Visia CR), wrinkle occupancy rate (Visia CR), and skin tone homogeneity (Visia CR). Subjective self-assessments of skin appearance were collected.

### Summary of Results

In this study, the following observations were made:

A skin regimen consisting of AA20 in combination with the Minimalist Hydrating Cream (MHC) led to significant improvements in multiple signs of skin aging, including wrinkling, dark spots, firmness, and elasticity.

Improvements in firmness, elasticity, and skin tone evenness were statistically significant by day 14 after treatment commenced.

All other measures, including improvements in dark spot intensity, skin brightness, and wrinkles and fine lines were statistically significant by day 30 after treatment commenced.

Results demonstrate that AA20 and MHC work synergistically as part of a daily regimen.

Participants rated the combined AA20+MHC regimen positively, with 100% of participants agreeing that their skin looked more youthful and healthier at the end of the study and would continue the use of these product.

### Results

Clinical grading
At day 14, there was a statistically significant improvement for firmness (visual) (p=0.003) and elasticity (tactile) (p<0.001).
At day 30, there was a statistically significant improvement across all measures, including wrinkles/fine lines (p=0.001), firmness (p<0.001), elasticity (p<0.001), skin tone homogeneity/evenness (p=0.003), and dark spots intensity (p=0.024).
At day 60, there was a statistically significant improvement across all measures, including wrinkles/fine lines (p<0.001), firmness (p<0.001), elasticity (p=0.002), skin tone homogeneity/evenness (p=0.001), and dark spots intensity (p=0.01).
At day 90, there was a statistically significant improvement across all measures, including wrinkles/fine lines (p<0.001), firmness (p<0.001), elasticity (p<0.001), skin tone homogeneity/evenness (p<0.001), and dark spots intensity (p=0.002).

### Instrumental Measurements

At 14 days, there was a statistically significant improvement in the active group for wrinkle visibility as measured by Visia CR (p<0.001), wrinkle occupancy rate as measured by Visia CR (p<0.001), skin tone homogeneity as measured by Visia CR (p=0.045).

At day 30, there was a statistically significant improvement in the active group for wrinkle visibility as measured by Visia CR (p<0.001), wrinkle occupancy rate (p<0.001), skin tone homogeneity as measured by Visia CR (p=0.03), skin brightness as measured by Chroma Meter (p=0.025), firmness as measured by Cutometer (p=0.001).

At day 60, there was a statistically significant improvement in the active group for wrinkle visibility as measured by Visia CR (p<0.001), wrinkle occupancy rate (p<0.001), skin tone homogeneity as measured by Visia CR (p=0.009), skin brightness as measured by Chroma Meter (p=0.015), firmness as measured by Cutometer (p=0.001).

At day 90, there was a statistically significant improvement in the active group for wrinkle visibility as measured by Visia CR (p<0.001), wrinkle occupancy rate (p<0.001), skin tone homogeneity as measured by Visia CR (p=0.012), skin brightness as measured by Chroma Meter (p=0.034), firmness as measured by Cutometer (p=0.019).

### Self-Assessment by Participants

After 14 days:
100% of participants agreed that the overall appearance of skin was improved.
100% of participants agreed that the skin tone was brighter.
100% of participants agreed that skin looked more youthful and healthier.

After 30 days of usage:
100% of participants agreed that brown spots appeared lighter.
100% of participants agreed that skin elasticity was improved.
100% agreed that skin tone evenness was improved.

After 60 days:
100% of participants agreed that radiance and clarity of skin were increased.

After 90 weeks of use:
100% of participants agreed that lines and wrinkles were less visible.

As shown in FIG. 9, a representative subject (subject 1) showed marked improvement in the appearance of wrinkles on the forehead, around the eyes, and around the mouth, hyperpigmentation on forehead and lower cheeks, and overall improvement in skin tone evenness at month 2.

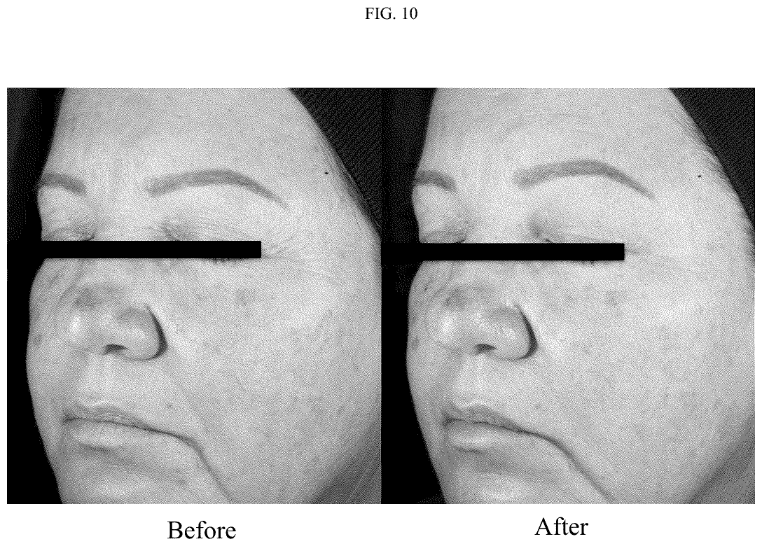

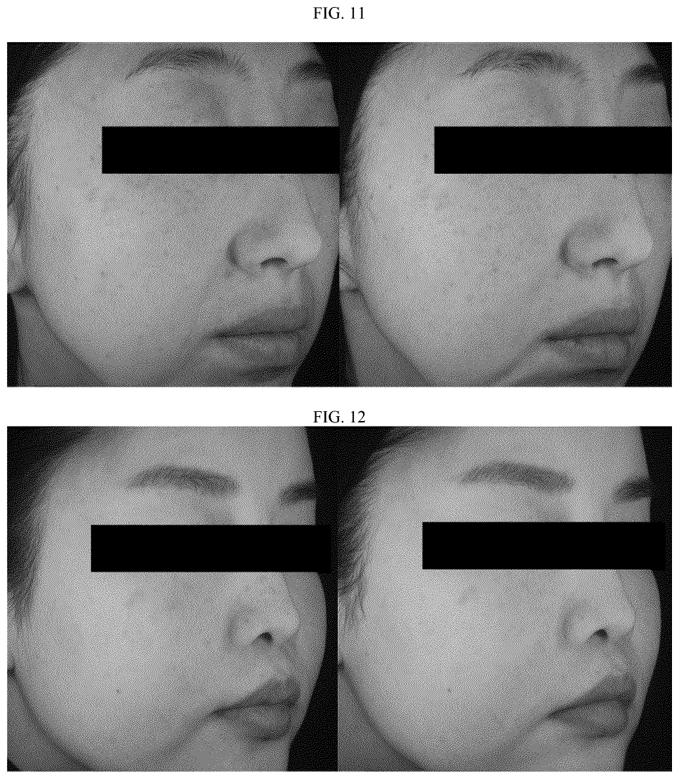

As shown in FIG. 10, another representative subject (subject 2) showed marked improvement in the appearance of wrinkling around the eyes, improvement in skin tone evenness, and overall brightening of skin tone at month 1.

### Conclusion

Improvement in measures of skin aging, including wrinkles/fine lines, firmness, and measures of hyperpigmentation, as measured by clinician grading and instrumentation were observed. Furthermore, assessments revealed subjective improvements in overall appearance of skin, uneven skin tone or discoloration, radiance, and clarity; reduced appearance of brown spots, reduced visibility of wrinkles/lines, improved skin texture, skin tone, skin youthfulness and health, firmness, elasticity, and skin tone homogeneity.

While the illustrative embodiments of the invention have been described with particularity, it will be understood that various other modifications will be apparent to, and can be readily made by those skilled in the art, without departing from the spirit and scope of the invention. Accordingly, it is not intended that the scope of the claims appended hereto be limited to the examples and descriptions set forth hereinabove but rather that the claims be construed as encompassing all the features of patentable novelty in the present invention, including all features which would be treated as equivalents by persons having ordinary skill in the art of formulating topically-applied personal care and dermatological products.

What is claimed is:

1. A storage stable topical emulsion composition, comprising:
a continuous phase that is at least 75% by weight in total of the composition, comprising:
a. a solvent comprising a mixture of water and an alkanediol; and
b. panthenol;
a dispersed phase that is at least 5% by weight in total, comprising:
a. a non-ethoxylated emulsifier at 0.5% to 3% by weight;
b. an emollient;
c. a barrier repairing agent; and
d. a soothing agent;
with the proviso that the composition comprises:
e. less than 0.5% by weight of oil-based stabilizers (e.g., fatty alcohol stabilizer, e.g., cetyl alcohol) and
f. less than 0.5% by weight of triglycerides; and
g. less than 0.5% by weight of ethoxylated emulsifiers.

2. The composition of claim 1, wherein the dispersed phase is at least 5% by weight in total.

3. The composition of any one of claims 1-2, wherein the dispersed phase is at least 10% by weight in total.

4. The composition of any one of claims 1-3, comprising 2% by weight of the non-ethoxylated emulsifier.

5. The composition of any one of claims 1-3, comprising 3% by weight of the non-ethoxylated emulsifier.

6. The composition of any one of claims 1-5, wherein the composition is storage stable and exhibits a viscosity ranging from 10,000 to 75,000 cps.

7. The composition of any one of claims 1-5, wherein the composition exhibits a pH between 3.5 and 6.5 (e.g., 5.0 to 6.0) when stored at 25° C.

8. The composition of any one of claims 1-5, wherein the composition exhibits a pH between 3.5 and 6.5 (e.g., 5.0 to 6.0) when stored at 30° C.

9. The composition of any one of claims 1-5, wherein the composition exhibits a pH between 3.5 and 6.5 (e.g., 5.0 to 6.0) when stored at 35° C.

10. The composition of any one of claims 1-9, wherein the composition comprises:
a. 55%-80% by weight of water;
b. 4% to 8% by weight of the alkanediol;
c. 3% to 10% by weight of panthenol;
d. 0.5% to 3% by weight of the non-ethoxylated emulsifier (e.g., lecithin, sodium acrylate copolymer, etc.);
e. 2%-18% by weight of the emollient;
f. 2%-10% by weight of the barrier repairing agent;
g. 0.1% to 1.5% by weight of the soothing agent;
with the proviso that the composition does not comprise an oil-based stabilizer or a triglyceride.

11. The composition of any one of claims 1-10, wherein the composition:
is storage stable against separation of continuous and dispersed phases;
is capable of application to the skin of a subject without pilling;
is capable of providing a cooling sensation on the skin of a subject;
exhibits a viscosity of 20,000-75,000 cps and pH between 5.0 and 6.5 when stored at 25° C. for 3 months.

12. The composition of any one of claims 1-10, wherein the composition exhibits a viscosity of 15,000-75,000 cps.

13. The composition of any one of claims 1-10, wherein the composition:
is storage stable against separation of continuous and dispersed phases;
is capable of application to the skin of a subject without pilling;
is capable of providing a cooling sensation on the skin of a subject;
exhibits a viscosity of 45,000-75,000 cps and pH between 5.0 and 6.0 when stored at 25° C. for 3 months.

14. The composition of any one of claims 1-13, wherein the alkanediol is selected from propanediol, propylene glycol, butylene glycol, pentylene glycol, hexanediol, caprylyl glycol, and a combination thereof.

15. The composition of claim 14, wherein the continuous phase comprises 1,2-hexanediol, and propanediol.

16. The composition of claim 14, wherein the continuous phase comprises propanediol and caprylyl glycol.

17. The composition of claim 14, wherein the alkanediol is 1,3 propanediol.

18. The composition of claim 16, wherein the composition comprises about 6%-7% by weight of 1,3 propanediol.

19. The composition of any one of claims 1-18, wherein the composition comprises 65% to 71% by weight of water.

20. The composition of any one of claims 1-19, wherein the composition comprises about 5% by weight of panthenol.

21. The composition of any one of claims 1-20, wherein the composition (e.g., continuous phase of the composition) further comprises a chelating agent.

22. The composition of claim 21, wherein the composition comprises 0.05% to 0.2% by weight of the chelating agent.

23. The composition of claim 21 or 22, wherein the chelating agent is phytic acid or EDTA, or a salt thereof (e.g., disodium EDTA, or sodium phytate).

24. The composition of claim 23, wherein the continuous phase comprises about 0.1% by weight of phytic acid solution (50% (w/w) in water).

25. The composition of any one of claims 1-25, wherein the barrier repairing agent is selected from c10-30 cholesterol/lanosterol esters, cholesteryl isostearate, cholesteryl chloride, cholesteryl nonanoate, lanolin, lanolin wax, lanolin oil, lanolin acid, lanolin alcohol, and a combination thereof.

26. The composition of claim 25, wherein the composition comprises about 4% by weight of C10-30 cholesterol/lanosterol esters.

27. The composition of any one of claims 1-26, wherein the emollient is selected from pentaerythritol tetraethylhexanoate, c12-15 alkyl benzoate, dipantaerythrityl pentaisononanoate, pentaerythrityl tetraisostearate, pentaerythrityl tetrabehenate, dipentaerythrityl pentaisononanoate, diisopropyl sebacate, isononyl isononanoate, squalane, and a combination thereof.

28. The composition of claim 27, wherein the composition comprises about 12% by weight of pentaerythrityl tetraethylhexanoate.

29. The composition of any one of claims 1-28, wherein the emulsifier is selected from lecithin, sodium acrylate copolymer, and a combination thereof.

30. The composition of claim 29, wherein the composition comprises a blend of lecithin and sodium acrylate copolymer.

31. The composition of claim 30, wherein the composition comprises about 2% by weight of the blend of lecithin and sodium acrylate copolymer.

32. The composition of any one of claims 1-31, wherein the soothing agent is selected from: centella asiatica extract, madecassoside, asiaticoside, glycyrrhetinic acid, and a combination thereof.

33. The composition of claim 32, wherein the composition comprises a blend of madecassoside, asiaticoside; and glycyrrhetinic acid.

34. The composition of claim 32, wherein the composition comprises about 0.14% by weight madecassoside and about 0.06% by weight asiaticoside.

35. The composition of claim 33, wherein the composition comprises about 0.2% by weight blend of madecassoside and asiaticoside, and about 0.5% by weight glycyrrhetinic Acid.

36. The composition of any one of claims 1-35, wherein the composition comprises a preservative, wherein the preservative is an antimicrobial agent.

37. The composition of claim 36, wherein the antimicrobial agent is caprylhydroxamic acid.

38. The composition of any one of claims 1-35, wherein the continuous phase further comprises caprylhydroxamic acid (e.g., about 0.05-0.15% by weight).

39. The composition of claim 38, wherein the continuous phase comprises caprylhydroxamic acid, 1,2-hexanediol, and propanediol.

40. The composition of any one of claims 1-37, wherein the continuous phase comprises about 0.05% by weight of caprylhydroxamic acid, about 0.30% by weight of 1,2-hexanediol, and about 6.65% by weight propanediol.

41. The composition of any one of claims 1-40, wherein the fatty alcohol stabilizer is selected from cetyl alcohol, cetearyl alcohol, behenyl alcohol, stearyl alcohol, and a combination thereof.

42. The composition of any one of claims 1-41, wherein the triglyceride is selected from: caprylic/capric triglyceride, argan oil, sunflower seed oil, rapeseed oil, olive oil, coconut oil, palm kernel oil, babassu oil, palm oil, apricot kernel oil, tallow, baobab oil, cocoa butter, andiroba seed oil, mango butter, avocado oil, cottonseed oil, marula oil, shea butter, grape seed oil, almond oil, safflower oil, moringa oil, and a combination thereof.

43. The composition of any one of claims 1-42, wherein the composition exhibits a pH ranging from 3.5 to 6.5 after storage for at least 3 months at 5° C.±2° C. in a multi-use container.

44. The composition of any one of claims 1-42, wherein the composition exhibits a pH ranging from 3.5 to 6.5 after storage for at least 3 months at 25° C.±2° C. in a multi-use container.

45. The composition of any one of claims 1-42, wherein the composition exhibits a pH ranging from 3.5 to 6.5 after storage for at least 3 months at 40° C.±2° C. in a multi-use container.

46. The composition of any one of claims 1-42, wherein the composition exhibits a pH ranging from 3.5 to 6.5 after storage for at least 3 months at 45° C.±2° C. in a multi-use container.

47. The composition of any one of claims 43-46, wherein the composition exhibits a viscosity ranging from 10,000 to 75,000 cps after storage for at least 3 months at 5° C.±2° C. in a multi-use container.

48. The composition of any one of claims 43-46, wherein the composition exhibits a viscosity ranging from 10,000 to 75,000 cps after storage for at least 3 months at 25° C.±2° C. in the multi-use container.

49. The composition of any one of claims 43-46, wherein the composition exhibits a viscosity ranging from 10,000 to 75,000 cps after storage for at least 3 months at 40° C.±2° C. in the multi-use container.

50. The composition of any one of claims 33-36, wherein the composition exhibits a viscosity ranging from 10,000 to 75,000 cps after storage for at least 3 months at 45° C.±2° C. in the multi-use container.

51. The composition of any one of claims 43-46, wherein the composition exhibits a viscosity ranging from 49,000 to 53,000 for at least 3 months at 5° C.±2° C. in the multi-use container.

52. The composition of any one of claims 43-46, wherein the composition exhibits a viscosity ranging from 49,000 to 55,000 for at least 3 months at 25° C.±2° C. in the multi-use container.

53. The composition of any one of claims 43-46, wherein the composition exhibits a viscosity ranging from 40,000 to 52,500 for at least 3 months at 40° C.±2° C. in the multi-use container.

54. The composition of any one of claims 43-46, wherein the composition exhibits a viscosity ranging from 40,000 to 51,500 for at least 3 months at 45° C.±2° C. in the multi-use container.

55. The composition of any one of claims 43-46, wherein the multi-use container is a glass bottle.

56. The composition of any one of claims 43-46, wherein the multi-use container comprises a plastic bottle with an airless pump.

57. A kit comprising the composition of any one of claims 1-56.

58. The kit of claim 57, further comprising instructions for using the kit.

59. The kit of claim 58, wherein the instructions for using the kit comprises:
(a) gently applying to the skin (e.g., face) ½ to 1 pump of the composition of any one of claims 1-57, wherein the composition is applied in the morning, in the evening, once daily, twice daily, or as needed.

60. A kit comprising:
i. a topical vitamin C formulation; and
ii. a topical minimalist hydrating formulation.

61. The kit of claim 60, wherein the vitamin C formulation comprises:
o. 5% to 28% by weight ascorbic acid;
p. 5% to 20% by weight of a urea agent;
q. 0.1% to 5% by weight of a cinnamic acid or derivative thereof; and
r. less than 10% by weight in total of one or more optional additional components;
s. dissolved in a non-aqueous skin-compatible solvent comprising polyol, C₍₂₋₆₎ alkanediol, glycol ether, dimethyl ether, or a combination thereof; wherein the ascorbic acid is dissolved at a concentration (AA) that is above its maximum concentration in the solvent alone (X), and the urea is dissolved at a concentration that is at least (AA-X)*1.25.

62. The kit of claim 60 or 61, wherein the minimalist hydrating formulation comprises:
a continuous phase that is at least 75% by weight in total of the composition, comprising:
t. a solvent comprising a mixture of water and an alkanediol; and
u. panthenol;
a dispersed phase that is at least 5% by weight in total, comprising:
v. a non-ethoxylated emulsifier at 0.5% to 3% by weight;
w. an emollient;
x. a barrier repairing agent; and
y. a soothing agent;
with the proviso that the composition comprises:
z. less than 0.5% by weight of oil-based stabilizers (e.g., fatty alcohol stabilizer, e.g., cetyl alcohol) and
aa. less than 0.5% by weight of triglycerides; and
bb. less than 0.5% by weight of ethoxylated emulsifiers.

63. The kit of claim 62, wherein the composition is storage stable and exhibits a viscosity ranging from 10,000 to 75,000 cps (e.g., 45,000+/−1,000 cps) and pH between 3.5 and 6.5 (e.g., 5.0 to 6.0) when stored at 25° C.

64. The kit of any one of claims 60-63, wherein the minimalist emulsion formulation comprises:
v. 55%-80% by weight of water;
w. 4% to 8% by weight of the alkanediol;
x. 3% to 10% by weight of panthenol;
y. 0.5% to 3% by weight of the non-ethoxylated emulsifier (e.g., lecithin, sodium acrylate copolymer, etc);
z. 2%-18% by weight of the emollient;
aa. 2%-10% by weight of the barrier repairing agent;
bb. 0.1% to 1.5% by weight of the soothing agent;
with the proviso that the composition does not comprise an oil-based stabilizer or a triglyceride.

65. The kit of any one of claims 60-64, wherein the kit further comprises instructions for using the minimalist hydrating formulation and the vitamin C formulation.

66. The kit of claim 65, wherein the instructions for using the minimalist hydrating formulation comprise: applying an effective amount of a topical minimalist hydrating formulation to an area of the skin.

67. The kit of any one of claims 65-66, wherein the instructions for using the vitamin C formulation comprise applying an effective amount of a vitamin C formulation to the same area of the skin for which the minimalist hydrating formulation was applied.

68. The kit of any one of claim 67, wherein the area of skin is selected from: face, arms, and legs.

69. The kit of any one of claims 65-68, wherein the instructions comprise:
(a) gently applying ½ to 1 pump of the vitamin C formulation to clean skin;
(b) applying the vitamin C formulation in the morning or evening, before or after the use of the minimalist hydrating formulation.

70. The kit of any one of claims 65-69, wherein the instructions further comprise using the vitamin C formulation 2-3 times per week, once daily, or initially 2-3 times per week until the skin is acclimated to the vitamin C formulation, followed by once daily.

71. The kit of any one of claims 65-70, wherein the instructions further comprise applying the minimalist hydrating formulation in the morning or evening, once daily or as needed.

72. The kit of any one of claims 65-71, wherein vitamin C formulation is used before applying the minimalist hydrating formulation.

73. The kit of any one of claims 65-71, wherein vitamin C formulation is used after applying the minimalist hydrating formulation.

74. The kit of any one of claims 60-73, wherein the vitamin C formulation comprises:
Propanediol, Ascorbic Acid, Urea, Ferulic Acid, Diglycerin, and Pinus Pinaster Bark Extract.

75. The kit of any one of claims 60-74, wherein the minimalist hydrating formulation comprises:
Water, Pentaerythrityl Tetraethylhexanoate, Propanediol, Panthenol, C10-30 Cholesterol/Lanosterol Esters, Sodium Acrylates Copolymer, Lecithin, Glycyrrhetinic Acid, Madecassoside, Asiaticoside, Phytic acid, Caprylhydroxamic acid, and 1,2-Hexanediol.

76. The kit of any one of claims 60-75, wherein the kit comprises two containers, wherein one container comprises the vitamin C formulation, and a second container comprises the minimalist hydrating formulation.

77. A method of reducing the appearance of skin aging, the method comprising:
i) applying an effective amount of a topical minimalist hydrating formulation of any one of claims 1-59 to an area of skin; and
ii) applying an effective amount of a vitamin C formulation to the same area of skin as the minimalist hydrating formulation;
wherein steps i) and ii) are carried out at least once daily to reduce the appearance of skin aging.

78. The method of claim 77, wherein the vitamin C formulation is applied immediately after or immediately before applying the minimalist hydrating formulation.

79. The method of claim 78, wherein the vitamin C formulation is applied 1 minute after applying the minimalist hydrating formulation, 2 minutes after applying the minimalist hydrating formulation, 3 minutes after applying the minimalist hydrating formulation, 4 minutes after applying the minimalist hydrating formulation, 5 minutes after applying the minimalist hydrating formulation, 6 minutes after applying the minimalist hydrating formulation, 7 minutes after applying the minimalist hydrating formulation, 8 minutes after applying the minimalist hydrating formulation, 9 minutes after applying the minimalist hydrating formulation, or 10 minutes after applying the minimalist hydrating formulation.

80. The method of any one of claims 77-79, wherein each of the minimalist hydrating formulation and the vitamin C formulation are applied at once daily, at least twice daily, or 2-3 times per week.
